(19) United States
(12) Patent Application Publication
(10) Pub. No.: US 2007/0117771 A1
(43) Pub. Date: May 24, 2007

(54) VGLUT-specific dsRNA compounds

(75) Inventors: Clemens Gillen, Aachen (DE); Gregor Bahrenberg, Aachen (DE); Thomas Christoph, Aachen (DE); Eberhard Weihe, Marburg (DE); Martin K.H. Schaefer, Marburg (DE); Florian Bender, Wuerzburg (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/518,284

(22) Filed: Sep. 11, 2006

Related U.S. Application Data
(63) Continuation of application No. PCT/EP05/01970, filed on Feb. 24, 2005.

(30) Foreign Application Priority Data
Mar. 10, 2004 (DE) 10 2004 011 687.3

(51) Int. Cl. A61K 48/00; C07H 21/02

(52) U.S. Cl. 514/44; 536/23.1

(57) ABSTRACT

VGLUT-specific dsRNAs capable of triggering the phenomenon of RNA interference, host cells containing theses dsRNAs, and pharmaceutical compositions containing these dsRNAs, in particular for the treatment of pain and other diseases associated with VGLUT family members.

### CROSS REFERENCE TO RELATED APPLICATIONS

This application is a continuation of international patent application no. PCT/EP2005/001970, filed Feb. 24, 2005, designating the United States of America, and published in German on Sep. 29, 2005 as WO 2005/090571, the entire disclosure of which is incorporated herein by reference. Priority is claimed based on Federal Republic of Germany patent application no. DE 10 2004 011 687.3, filed Mar. 10, 2004.

### BACKGROUND OF THE INVENTION

The invention relates to small, in particular interference-triggering, double-stranded RNA molecules (dsRNA), which are directed against members of the VGLUT family, and to host cells containing dsRNA according to the invention. dsRNA according to the invention and corresponding host cells are suitable as pharmaceutical compositions and for the production of pharmaceutical compositions, in particular for the treatment of pain and other diseases associated with VGLUT family members or the non-physiological expression thereof.

As defined by the International Association for the Study of Pain (IASP), pain is “an unpleasant sensory and emotional experience associated with acute or potential tissue damage, or described in terms of such damage” (Wall and Melzack, 1999). The organism reacts to a painful (nociceptive) stimulus with a complex reaction, in which sensory/discriminatory, cognitive, effective, autonomous and motor components participate. Whereas acute pain involves a physiological protective reaction and is vital to the survival of an individual, chronic pain, on the other hand, does not have a clear biological function. Nociceptive pain is triggered by noxious stimuli, such as heat, mechanical stimulation, protons or coldness, on specialized high-threshold sensory apparatus, the nociceptors, and is conveyed into the posterior horn of the spinal cord in the form of electrical activity by unmyelinated C-fibres or weakly myelinated Aδ fibres. The nociceptors are equipped with specific receptors and ion channels for this purpose (Scholz and Woolf, 2002).

Damaged or injured tissue, inflammation or tumour cells may be signals of nociceptors; they lead to the release of chemical mediators from inflamed cells, blood vessels and from the afferent terminals, which either themselves lead to activation of the nociceptors (for example by bradykinin) or alter the stimulus response behaviour of nociceptive afferent nerve fibres, for example by lowering the activation threshold (for example by prostaglandins, interleukins, NGF), and thus lead to sensitisation of the nociceptors (Sholz and Woolf, 2002).

The intensity of pain is coded by the number of impulses per unit time. In the case of nociceptive peripheral stimulation, the rapid synaptic response components (5 to 20 msec) and monosynaptic reflex responses in the spinal cord are brought about by AMP-kainate receptors, whereas NMDA and metabotrope glutamate receptors participate in particular in late, longer lasting (20 to 150 msec), polysynaptically mediated response components (Tölle, 1997).

The release of glutamate in the posterior horn of the spinal cord plays a decisive part in the creation of chronic pain (Baranauskas and Nistri, 1998; Zhuo, 2001). The release of glutamate leads to activation of the glutamate receptors (AMPA, kainite, mGlu-R, NMDA). Proteins which participate in the release of glutamate therefore represent interesting targets for pain research.

Glutamate is one of the most important excitatory neurotransmitters in the nervous system of vertebrates. The nonessential amino acid, glutamate, cannot breach the blood-nerve barrier and is therefore synthesized in the brain from glucose and a large number of other precursors.

The formation of glutamate in the excitatory nerve endings is catalysed by the enzyme, phosphate-activated glutaminase (PAG), from glutamine. The vesicular glutamate transporter (VGLUT) packs the glutamate in vesicles and releases the glutamate after arrival of a depolarized action potential by the influx of calcium from the vesicles into the synaptic cleft. From the synaptic cleft, the glutamate is transported in part by a plasma membrane transporter for excitatory amino acids (EAATs) back into the excitatory terminals, where it is packed in vesicles again.

Therefore, transporter proteins from two superfamilies participate in the transport of glutamate: plasma membrane transporters and the vesicular membrane transporters (Disbrow et al., 1982; Shioi et al., 1989; Tabb et al., 1992).

Three vesicular glutamate transporters, VGLUT1 (SLC17A6), VGLUT2 (SLC17A7) and VGLUT3 (SLC17A8), have been identified as members of the SLC-17 transporter family (type I phosphate/vesicular glutamate transporter), which induces the transport of organic anions, and have initially been identified as Na⁺-dependent phosphate transporters. VGLUT1, VGLUT2 and VGLUT3 bear the collective name of VGLUT. The proteins of the SLC17 family are expansive transmembrane proteins with 6 to 12 hypothetical transmembrane domains, the three aforementioned glutamate transporters occurring as the subfamily of the SLC17 family. The three aforementioned VGLUTs are highly homologous with one another in their amino acid sequence (Takamori et al., 2002). The cDNA sequence of human VGLUT2 appears under gene bank accession number NM_{—}020346 in the databases. The amino acid sequence of human VGLUT2 appears under NP_{—}065079 in the databases. The cDNA sequence of VGLUT2, rat, appears under NM_{—}053427 in the databases. The amino acid sequence of VGLUT2, rat, appears under NP_{—}445879 in the databases. The cDNA sequence of VGLUT2, mouse, appears under AN: BC038375 in the databases, the amino acid sequence of VGLUT2, mouse, appears under AAH38375 in the databases.

The cortical layers of the cerebrum have a pronounced mRNA expression level for VGLUT1, whereas VGLUT2 mRNA could be detected, in particular, in layer IV of the cortex. VGLUT3 expression is localized, for example, in the inhibitory cells in layer II of the parietal cortex, or in GAD-positive interneurons in the Stratum radiatum of CA1-CA3 of the hippocampus. Furthermore, VGLUT1 and VGLUT2 could only be detected in the nerve endings, whereas VGLUT3 was detected not only in the synaptic vesicles but also in vesicular structures of astrocytes and neuronal dendrites (Fremeau et al., 2002).

VGLUT1 and VGLUT2 are expressed in two separate populations in the spinal ganglion, a third subpopulation having coexpression for VGLUT1 and VGLUT2. VGLUT2-mRNA is expressed predominantly by small and medium DRG neurons, whereas VGLUT1-mRNA is expressed by medium and large DRG neurons (posterior root fibre ganglion). In isolated cases, VGLUT3-mRNA-expressing neurons can also appear in the spinal ganglion (Oliveira et al., 2003; Todd et al, 2003).

Both VGLUT1 and VGLUT2 can be detected at protein level in the grey matter of the spinal cord (Varoqui et al., 2002). The dominance of VGLUT2 in the superficial posterior horn is evidence of a prominent role in pain transmission. The dominance of VGLUT1 in the deep posterior horn is evidence of a role in proprioception. Therefore, VGLUT2, in particular, but also VGLUT1 is a pain target (Varoqui et al., 2002).

The effective treatment of pain is a great challenge in molecular medicine. Acute and transitory pain is an important signal from the body to protect people from serious damage by the environment or overloading of the body. On the other hand, chronic pain, which lasts longer than the cause of the pain and the expected duration of healing, has no known biological function and affects hundred of millions of humans throughout the world. Approximately 7.5 million people suffer from chronic pain in the Federal Republic of Germany alone. Unfortunately, the pharmacological treatment of chronic pain is still unsatisfactory and therefore remains a challenge in current medical research. Frequently, currently existing analgesics are not sufficiently effective and sometimes have serious side effects.

Due to their function and expression profile, VGLUT proteins in general, but VGLUT2 in particular, represent an interesting starting point as a target for new pain remedies (Varoqui et al., 2002).

Some substances which are capable of modulating the activity or expression of VGLUT are known, for example, from research into pain relief (Carrigan et al. 2002; Roseth et al., 1995; Roseth et al., 1998). However, they do not act subtype-specifically, and therapeutic formulations would be limited both by the availability of the inhibitors in the nervous system and at the synaptic vesicles and by the nonspecific effect on all three VGLUTs.

### SUMMARY OF THE INVENTION

An object of the present invention is to provide further substances, which are capable of selectively and efficiently modulating the effect of the VGLUTs, for example VGLUT1, VGLUT2 and VGLUT3.

Another object of the invention is to provide VGLUT-modulating substance which optionally exhibit cell permeability.

A further object of the invention is to provide VGLUT-modulating substances which are usable for therapeutic purposes, in particular for the treatment of pain.

These and other objects are achieved by the present invention as described and claimed hereinafter. According to the invention, the object is achieved by VGLUT-specific dsRNAs that are capable of triggering the phenomenon of RNA interference.

Double-stranded RNA (dsRNA) according to the invention contains a sequence with the general structure 5′-(N₁₇₋₂₅)-3′, wherein N is any base and represents nucleotides. The general structure consists of a double-stranded RNA with a macromolecule made up of ribonucleotides, wherein the ribonucleotide consists of a pentose (ribose), an organic base and a phosphate. The organic bases in the RNA consist of the purine bases, adenine (A) and guanine (G), and the pyrimidine bases, cytosine (C) and uracil (U). The dsRNA contains nucleotides with a directed structure with overhangs. Double-stranded RNAs according to the invention of this type can trigger the phenomenon of RNA interference (siRNAs).

The phenomenon of RNA interference as an immunological defence system was noticed during immunological research into higher eukaryotes.

The system was originally described in various species independently of one another, initially in C. elegans (Fire et al., 1998), before the underlying mechanisms could be identified as identical at specific levels of the processes: RNA-mediated virus resistance in plants (Lindbo and Dougherty, 1992), PTGS (post-transcriptional gene silencing) in plants (Napoli et al., 1990) and RNA interference in eukaryotes are accordingly based on a common mode of operation (Plasterk, 2002).

The in vitro technique of RNA interference (RNAi) is based on double-stranded RNA molecules (dsRNA) which trigger the sequence-specific suppression of gene expression (Zamore (2001) Nat. Struct. Biol. 9: 746-750; Sharp (2001) Genes Dev. 5:485-490: Hannon (2002) Nature 41: 244-251). The activation of protein kinase R and RNaseL brought about nonspecific effects such as an interferon response (Stark et al. (1998) Annu. Rev. Biochem, 67: 227-264; He und Katze (2002) Viral Immunol. 15: 95-119) during the transfection of mammalian cells with long dsRNA. These nonspecific effects are obviated by using smaller, for example 21 to 23-type so-called dsRNA (small interfering RNA), as nonspecific effects are not triggered by dsRNA that is smaller than 30 bp (Elbashir et al. (2001) Nature 411: 494-498). dsRNA molecules have also been used recently in vivo (McCaffrey et al. (2002), Nature 418: 38-39; Xia et al. (2002), Nature Biotech 20: 1006-1010; Brummelkamp et al. (2002), Cancer Cell 2: 243-247.

dsRNAs that are directed against members of the VGLUT family are disclosed in the context of the present invention. According to the invention, these dsRNAs may be of various categories. dsRNA according to the invention may exist in the form (i) of an siRNA, (ii) of a long dsRNA containing one or more identical or different siRNA(s) in the long dsRNA sequence, (iii) of an siRNA-based hairpin RNA or (iv) of a miRNA-based hairpin siRNA. All the aforementioned embodiments are covered by the term “dsRNA”.

Although (i) siRNA (typically chemically synthesized and then incorporated into the RISC complex intracellularly while bypassing the dicing step, so sequence-specific mRNA degradation (of the target sequence) takes place) is described in more detail hereinafter, (ii), in other words a long dsRNA (ds: double-stranded), is a precursor of siRNA (according to (i)), which is only processed into pure siRNA by a dicing step (enzyme: dicer). This precursor of siRNA, which is typically converted only intracellularly into mature siRNA, meets the requirements for use, for example, as a pharmaceutical composition or for production of a pharmaceutical composition for the treatment of the indications mentioned in the present application. In a preferred embodiment, a plurality of different siRNAs, which may differ in efficiency, are formed according to the invention in this respect, in other words from largerVGLUT-dsRNA molecules, in particularVGLUT1-, 2- or 3-specific dsRNA molecules (preferably >30 bp, more preferably >40 bp and even more preferably >50 bp), after dicer processing. Long (optionally hairpin-shaped) dsRNA molecules, which are transformed intracellularly into various siRNAs after dicer processing may also be expressed in a cell on a vector basis (apart from chemical synthesis) under the control of a Pol II promoter. The Pol II promoter allows inducible tissue- or cell type-specific expression (Kennerdell and Carthew, 2000). This form of application therefore allows inducible transient simultaneous expression of a large number of siRNAs, which originate from a precursor dsRNA. According to a further particularly preferred embodiment, dsRNA molecules according to the invention of this type may form a specific phenotype by genetic manipulation techniques such as homologous recombination of stem cells.

Modifications of siRNA according to (i), namely siRNA-based hairpin bends (iii), may also be used according to the invention. Hairpins of this type can preferably occur at one end, but optionally also at both ends of the siRNA double strand. There are typically at least 5, more preferably at least 8 and even more preferably at least 10 nucleotides, which, owing to modifications and/or in the absence of corresponding complementarity, do not form double-stranded interactions, but form a bend which initially bonds the two strands together covalently. An siRNA-based hairpin RNA of this type may be further processed into active siRNA by corresponding enzymes (for example dicers), for example intracellularly. Finally, (iv), miRNA-based hairpin RNAs directed against sequences of the VGLUT family, also form part of the present invention. These are imperfectly complementary siRNAs, preferably with at least one hairpin at the terminus (at the termini). Imperfectly complementary duplex strands of mRNA of this type comprise at least one defective conjugation, preferably between 1 and 4 defective conjugations in the duplex strand. The effect of miRNA-based hairpin RNA is based on the enzymatic processing thereof (for example by dicers) to miRNA(s), subsequent incorporation thereof into miRNPs and finally the translation inhibition thereof.

The respective length of the duplex strands in embodiments (i), (iii) and (iv) according to the invention does not differ and all embodiments can be chemically synthesized.

dsRNAs according to the invention preferably have the general structure 5′-(N₁₉₋₂₅)-3′, more preferably 5′-(N₁₉₋₂₄)-3′, even more preferably 5′-(N₂₁₋₂₃)-3′, where N is any base. At least 90%, preferably 99% and, in particular 100% of the nucleotides of a dsRNA according to the invention may be complementary to a fragment of the (m)RNA sequence of a member of VGLUT family, in particularVGLUT1, VGLUT2 or VGLUT3. 90% complementary means that, for example, with a given length of 20 nucelotides of a dsRNA according to the invention, it is not complementary with the corresponding fragment on the (m)RNA in the case of at most 2 nucleotides. The sequence of the double-stranded RNA, with its general structure, is preferably completely complementary with a fragment of the (m)RNA of a member of the VGLUT family, in particularVGLUT1, VGLUT2 or VGLUT3.

VGLUT-dsRNA comprising the following sequence patterns are also preferred: AAN₁₉TT, NAN₁₉NN, NARN₁₇YNN and/or NANN₁₇YNN, wherein N represents any nucleotide, A represents adenosine, T represents thymidine, R represents purines (A or G) and Y represents pyrimidine bases (C or T).

A dsRNA according to the invention can basically be complementary with any desired fragment on the mRNA or the primary transcript of a member of the VGLUT family.

In a eukaryotic cell, the gene is transcribed over its entire length, including both introns and exons, into a long RNA molecule, the primary transcript, to produce an mRNA. The stability of the cellular mRNA is ensured by processing the primary transcript at the 5′ end with an addition of an untypical nucleotide having a methylated guanine and polyadenylation at the 3′ end. Before the RNA leaves the cell nucleus, the intron sequences are removed and the exons spliced together by RNA splicing.

Both the primary transcript and the processed mRNA may be target sequences for dsRNA according to the invention. The primary transcript and the mRNA are described hereinafter as (m)RNA for short.

Basically any 17 to 29, preferably 17 to 25, base pair long fragments occurring in the encoding region of the (m)RNA can serve as the target sequence for a dsRNA according to the invention. Target sequences for dsRNAs according to the invention which lie between position 70 and 1730 (calculated from the respective AUG starting triplet of the encoding region of the (m)RNA of human VGLUT2 or VGLUT3), preferably between 100 and 1500 and quite particularly preferably between 600 and 1200 are also particularly preferred. Therefore, 17 to 25, in particular 19 to 25 and quite particularly 21 to 23, base pair long fragments on the (m)RNA, of which the starting nucleotide corresponds to a nucleotide of a position 80 to 1600 (or the aforementioned further preferred regions) of the encoding region of the VGLUT2- or 3-(m)RNA and of which the terminal nucleotide lies 17 to 25, preferably 19 to 25 and quite particularly preferably 21 to 23 nucleotides further downstream from the respective initiating nucleotide, are preferred.

With respect to VGLUT1, target sequences of the encoding region are similarly particularly preferred, in particular target sequences lying between position 600 and 1200 of the encoding region (calculated from the AUG initiating triplet).

dsRNAs which are directed against regions in the encoding region (m)RNA of a member of the VGLUT family are particularly preferred. In particular, dsRNAs according to the invention of this type, which are located in the central area of the encoding region, preferably at least 50, 70, 100 nucleotides removed from the AUG initiating triplet of the (m)RNA or at least 50 nucleotides, preferably at least 70, and more preferably 100 nucleotides removed from the 3′-terminal encoding region of the (m)RNA, should be directed against VGLUT-(m)RNA fragments.

Particularly preferred are dsRNAs according to the invention, in particular siRNAs which are directed against fragments in the encoding region of the VGLUT1, 2 or 3 (m)RNA (or cDNA), which begin with the initiating sequence AA.

dsRNAs according to the invention, in particular siRNAs which are directed against fragments in the encoding region of the VGLUT1-(m)RNA (or cDNA) are more particularly preferred; dsRNAs according to the invention, in particular siRNAs, which are directed against the sequences AACGTGCGCAAGTTGATGAAC (SEQ ID NO: 14) or AAGTTGATGAACTGCGGAGGC (SEQ ID NO: 14), are additionally preferred.

With respect to dsRNAs according to the invention, in particular siRNAs against VGLUT2, dsRNAs, in particular siRNAs are more particularly preferred, which are complementary and therefore directed against (m)RNA-fragments (or cDNA) which, for example, comprise the sequence AATGCCTTTAGCTGGCATTCT (SEQ ID NO: 16), AATGGTCTGGTACATGTTTTG (SEQ ID NO: 17), AAAGTCCTGCAAAGCATCCTA (SEQ ID NO: 18), AAGAACGTAGGTACATAGAAG (SEQ ID NO: 20), AATTGTTGCAAACTTCTGCAG (SEQ ID NO: 21), AAATTAGCAAGGTTGGTATGC (SEQ ID NO: 22), AATTAGCAAGGTTGGTATGCT (SEQ ID NO: 23), AAGGTTGGTATGCTATCTGCT (SEQ ID NO: 24), AAGCAAGCAGATTCTTTCAAC (SEQ ID NO: 25), AATGGGCATTTCGAATGGTGT (SEQ ID NO: 27), AATAAGTCACGTGAAGAGTGG (SEQ ID NO: 28), AATATTTGCCTCAGGAGAGAA (SEQ ID NO: 31), AAGTCTTATGGTGCCACAACA (SEQ ID NO: 32), AAGACTCACATAGCTATAAGG (SEQ ID NO: 34), and preferably AAGTCCTGCAAAGCATCCTAC (SEQ ID NO: 19), AACCACTTGGATATCGCTCCA (SEQ ID NO: 26), AAGTCACGTGAAGAGTGGCAG (SEQ ID NO: 29), AAGAGTGGCAGTATGTCTTCC (SEQ ID NO: 30), and/or AATGGAGGTTGGCCTAGTGGT (SEQ ID NO: 33).

Quite particularly preferred are those dsRNAs according to the invention, in particular siRNAs, which are directed against fragments in the encoding region of the VGLUT3-(m)RNA (or cDNA), more preferably in turn those dsRNAs according to the invention, in particular siRNAs, which are directed against AATCTTGGAGTTGCCATTGTG (SEQ ID NO: 35), AATTCCAGGTGGTTTCATTTC (SEQ ID NO: 38), AACATCGACTCTGAACATGTT (SEQ ID NO: 39), AAGAGGTCTTTGGATTTGCAA (SEQ ID NO: 41), AATAAGTAAGGTGGGTCTCTT (SEQ ID NO: 42), AATCGTTGTACCTATTGGAGG (SEQ ID NO: 45), AAGAATGGCAGAATGTGTTCC (SEQ ID NO: 47), AATCATTGACCAGGACGAATT (SEQ ID NO: 48), AACTCAACCATGAGAGTTTTG (SEQ ID NO: 49), AAAGAAGATGTCTTATGGAGC (SEQ ID NO: 50), and/or AAGAGCTGACATCCTACCAGA (SEQ ID NO: 52), and preferably AACCGGAAATTCAGACAGCAC (SEQ ID NO: 36), AAACAGTGGGCCTTATCCATG (SEQ ID NO: 37), AAGGTTTAGTGGAGGGTGTGA (SEQ ID NO: 40), AAGTAAGGTGGGTCTCTTGTC (SEQ ID NO: 43), AAGGTGGGTCTCTTGTCAGCA (SEQ ID NO: 44), and/or AAGACCCGTGAAGAATGGCAG (SEQ ID NO: 46).

Preferably, (double-stranded) siRNAs according to the invention or suitable molecules of the other embodiments will comprise the sequence TT at the terminus of at least one strand, preferably in an overhanging manner relative to the terminus of the complementary other strand. The complementary other strand of the siRNA according to the invention then typically corresponds in its sequence at a terminus to the, for example aforementioned, sequences after AA (wherein T, in contrast to the foregoing target sequences, is replaced by U in the siRNA according to the invention) and at the other terminus typically has an overhanging TT (see also embodiment 4).

However, dsRNAs according to the invention could also be directed against nucleotide sequences on the VGLUT1, VGLUT2, VGLUT3-(m)RNA, which do not lie in the encoding region, in particular in the non-encoding 5′ region of the (m)RNA, of the regulating functions.

The boundaries at the 5′ end of the target sequence with, for example, AA of the nucleotide bonds are also reflected in the associated dsRNA according to the invention in a sequence 5′-AAN₁₅₋₂₃ (with the strand 3′-TTN₁₅₋₂₃ which is complementary therewith). A strand of the double-stranded RNA is therefore complementary with the primary or processed RNA transcript of the VGLUT1, 2 or 3 gene.

Preferably, however, effective blocking and cleavage of the (m)RNA of a member of the VGLUT family is achieved in particular in that certain rules of selection are adhered to when selecting the target sequence of dsRNAs according to the invention.

A particularly preferred embodiment of the present invention is a dsRNA which has a GC content of at least 30%, of 30 to 70% in a more preferred embodiment, and from 40% to 60% in a more preferred configuration, or even more preferably between 45% and 55%.

A further particularly preferred embodiment of the present invention is a target sequence which contains the same frequency of all nucleotides on the antisense strand. Finally, it is particularly preferred if 2′-deoxythymidine appears for the 2-nt 3′ overhang in an siRNA according to the invention or suitable dsRNA molecules of further embodiments, as it is thus protected from exonuclease activity. It is further preferred if the target sequence of a dsRNA according to the invention appears only once in the target genes or is also singular for the respective genome of the treated cells.

Combinations of the aforementioned properties in dsRNAs according to the invention are also particularly preferred.

dsRNAs according to the invention, which are not directed against binding points for proteins which bind to a VGLUT(m)RNA, are also quite particularly preferred. In particular, a dsRNA according to the invention should not be directed against those regions on a VGLUT-(m)RNA which relate, for example, to the 5′-UTR-region, the 3′-UTR-region (respective regions at which the splicing process takes place), an initiating codon and/or exon/exon transitions. It is also preferred if the target region on the (m)RNA, to which the dsRNA according to the invention binds, does not have monotonic or repetitive sequences, in particular fragments with poly-G-sequences. Target sequences in intron regions are also preferably avoided in the complementary dsRNA according to the invention, as RNAi is a cytoplasmatic process.

A modified nucleotide can preferably appear in a dsRNA according to the invention. According to the invention, the term “modified nucleotide” means that the respective nucleotide is chemically modified. By the term “chemical modification”, the person skilled in the art understands that the modified nucleotide is altered by replacement, attachment or removal of individual or a plurality of atoms or atom groups in comparison with naturally occurring nucleotides. At least one modified nucleotide in dsRNA according to the invention serves, on the one hand, for stability and, on the other hand, to prevent dissociation.

Preferably between 2 and 10, and quite particularly preferably between 2 and 5, nucleotides are modified.

The ends of the double-stranded RNA (dsRNA) can preferably be modified to counteract degradation in the cell or dissociation into the individual strands, in particular to prevent premature degradation by nucleases.

Dissociation of the individual strands of dsRNA, which is generally undesirable, occurs, in particular, when using low concentrations or short chain lengths. For particularly effective inhibition of dissociation, the nucleotide pair-mediated cohesion of the double-stranded structure of dsRNA according to the invention may be increased by at least one, preferably a plurality, in particular 2 to 5, chemical linkages. A dsRNA according to the invention, of which the dissociation is reduced, has higher stability to enzymatic and chemical degradation in the cell and in the organism or ex vivo.

The chemical linkage of the individual strands of a dsRNA according to the invention is advantageously formed by a covalent or ionic bond, hydrogen bridge bond, hydrophobic interaction, preferably van der Waals or stacking interactions or by metal ion coordination. According to a particularly advantageous configuration, it may be produced at least at one, preferably at both, ends. It has also proven to be advantageous that the chemical linkage is formed by means of one or more groups of compounds, the groups of compounds preferably being poly-(oxyphosphinicooxy-1,3-propane-diol) and/or polyethyleneglycol chains. The chemical linkage may also be formed by purine analogues used in the double-stranded structure, instead of purines. A further advantage is that the chemical linkage is formed by azabenzene units introduced in the double-stranded structure. It may also be formed by branched nucleotide analogues used in the double-stranded structure, instead of nucleotides.

It has proven advantageous to produce the chemical linkage using at least one of the following groups: methyl blue; bifunctional groups, preferably bis-(2-chorethyl)-amine; N-acetyl-N′-(p-glyoxybenzoyl)-cystamine; 4-thiouracil; psoralene. The chemical linkage may further be formed by thiophosphoryl groups arranged at the ends of the double-stranded region. The chemical linkage is preferably produced by triple helical bonds at the ends of the double-stranded region. The chemical linkage may advantageously be induced by ultraviolet light.

Modification of the nucleotides of the dsRNA leads to deactivation of a protein kinase (PKR) dependent on (double-stranded) RNA, in the cell. The PKR induces apoptosis. Advantageously, at least one 2′ hydroxy group of the nucleotides of the dsRNA in the double-stranded structure is replaced by a chemical group, preferably a 2′-amino or a 2′-methyl group. At least one nucleotide in at least one strand of the double-stranded structure may also be what is known as a locked nucleotide with a sugar ring which is preferably chemically modified by a 2′-O, 4′-C-methylene bridge. Advantageously, a plurality of nucleotides are locked nucleotides.

Modification of the nucleotides of dsRNA according to the invention affects, in particular, the dissociation of the nucleotides by reinforcing hydrogen bridge bonding. The stability of the nucleotides is increased and protected from an attack by RNAs.

A further method of preventing premature dissociation of dsRNA according to the invention in the cell involves the formation of the hairpin bend. In a preferred embodiment, a dsRNA according to the invention has a hairpin structure, to slow down the dissociation kinetics. With a structure of this type, a loop structure is preferably formed at the 5′- and/or 3′-end. A loop structure of this type does not have hydrogen bridges.

In addition, premature degradation may be prevented by modifying the backbone of the dsRNA according to the invention. dsRNA which is modified (for example, phosphorus thioate, 2′-O-methyl-RNA, LNA, LNA/DNA gapmers) and therefore has a longer half-life in vivo is particularly preferred.

A dsRNA according to the invention is preferably derived against the (m)RNA of the VGLUT family, in particular from VGLUT1, VGLUT2 and/or VGLUT3, from mammals, such as humans, monkeys, rats, dogs, cats, mice, rabbits, guinea pigs, hamsters, cattle, pigs, sheep and goats.

A dsRNA according to the invention preferably suppresses the expression of VGLUT1, VGLUT2 and/or VGLUT3 in the cell by at least 50%, 60%, 70%, particularly preferably to at least 90%; the dsRNAs according to the invention are therefore, in particular, suitable dsRNA molecules of the embodiments according to the invention, in other words (i) siRNA or (ii) long dsRNA or (iii) siRNA-based hairpin RNA or (iv) miRNA-based hairpin RNA, which can trigger the phenomenon of RNA interference. Suppression can be measured via a Northern blot, quantitative real time PCR or at protein level with VGLUT1-, VGLUT2-or VGLUT3-specific antibodies.

dsRNAs according to the invention, in particular human dsRNAs according to the invention, can have what are known as blunt ends, but also overhanging ends.

Overhanging ends can basically comprise at least two overhanging nucleotides, preferably 2 to 10, in particular 2 to 5, overhanging nucleotides at the 3′-terminus, optionally however also alternatively at the 5′-terminus.

dTdT at the respective 3′-terminus of the double-stranded dsRNA according to the invention, in particular siRNA, are preferred for the overhanging ends. As mentioned above, the overhanging nucleotides may be dT (deoxythymidine) or also uracil, but any overhanging ends can basically be attached to the dsRNA double strands according to the invention that are complementary with mRNA of VGLUT1, 2 or 3.

dsRNAs according to the invention, in particular siRNAs, may be directed against human VGLUT sequences or sequences of mammals, for example rats, pigs or mice or of domestic animals.

In the case of rat VGLUT sequences, preferred embodiments of the dsRNA according to the invention are directed against a target sequence of the VGLUT2-mRNA of the rat, which in a preferred embodiment is the (m)RNA-target sequence 5′-AAG GCU CCG CUA UGC GAC UGU-3′ (SEQ ID NO: 70) (the sequence corresponds at the level of the cDNA, although U is replaced by T). A particularly preferred dsRNA of the present invention is therefore a duplex molecule of which the sense strand has the sequence 5′-GGC UCC GCU AUG CGA CUG UTT-3′ (SEQ ID NO: 71) (i.e. with an overhanging TT at the 3′-terminus) and of which the antisense strand has the sequence 5′-ACA GUC GCA UAG CGG AGC CTT-3′ (SEQ ID NO: 72) (also with a TT which overhangs relative to the sense strand at the 3′-terminus). This ds molecule according to the invention is directed against the aforementioned fragment of the VGLUT2-mRNA. A further particularly preferred embodiment of the dsRNA according to the invention is directed against a different target sequence of the VGLUT2-mRNA (of the rat), namely against 5′-AAG CAG GAU AAC CGA GAG ACC-3′ (SEQ ID NO: 86). The two strands of a double-stranded siRNA according to the invention then typically contain the following sequences: 5′-r(GCAGGAUAACCGAGAGACC)dTT-3′ (SEQ ID NO: 87) (sense strand) and 5′-r(GGUCUCUCGGUUAUCCUGC)d(TT)-3′ (SEQ ID NO: 88) (antisense strand) or consist thereof.

The dsRNA is produced by processes known to the person skilled in the art by synthesizing nucleotides, in particular also oligonucleotides, for example by Merryfield synthesis, on an insoluble support (H. G. Gassen, Chemical and Enzymatic Synthesis of Gene Fragments (Verlag Chemie. Weinheim 1982)) or by a different method (Beyer/Walter, Lehrbruch der Organischen Chemie, 20th edition, (S. Hirzel Verlag, Stuttgart 1984), p. 816 ff.). VGLUT-mRNA may be obtained by hybridization using genome and cDNA databases. dsRNA molecules according to the invention, in particular siRNA molecules may, for example, be produced synthetically and optionally also obtained from various suppliers, for example IBA GmbH (Göttingen, Germany).

Double-stranded RNA according to the invention (dsRNA) may be enclosed in micellar structures which influence the separation of groups of substances in vitro and in vivo. The dsRNA preferably occurs in liposomes. Liposomes are artificial membranes, which are spherically closed in on themselves, of phospholipids in which hydrophilic substances are encapsulated in the aqueous interior and lipophilic substances may also be incorporated in the internal region of the lipid membrane. To be used for experimental or therapeutic purposes, liposomes have to be compatible with cells and tissues. The dsRNA, which is preferably present in the liposomes, may be modified by a peptide sequence, preferably by a lysine and arginine-rich sequence, for example a sequence of the viral TAT protein (for example containing AS 49-57) and then breach the cell membrane more easily as a transporter peptide.

The dsRNA can similarly be enclosed in viral natural capsids or in chemically or enzymatically produced artificial capsids or structures derived therefrom. The aforementioned features allow the dsRNA to be funnelled into predetermined target cells.

A further preferred subject of the present invention is a configuration of the VGLUT-dsRNAs according to the invention, which is an alternative to siRNA, namely as microRNAs (loc. cit.) with at least one hairpin bend, by means of which the two imperfectly complementary strands are covalently bound to one another (miRNA-based hairpin RNA) (cf. also Schwarz et al., 2002). These VGLUT-miRNAs according to the invention are transcribed by the cells themselves and, after sequence-specific binding to the mRNA, do not lead to mRNA degradation but merely induce translation repression. VGLUT-miRNAs according to the invention are transcribed as at least 50, preferably between 60 and 80, quite particularly preferably between 65 and 75 nucleotide-long precursors and form a characteristic “hairpin structure”. The enzyme dicer cuts, from these precursors in the cell, a 21 to 23 nucleotide-long double-stranded region that is unwound in further steps. Therefore, the mature miRNA can be incorporated, for example, into miRNP particles. These particles may then induce specific translation repression of the complementary mRNA. The degree of complementarity to the target mRNA decides whether the DNA duplex formed acts as miRNA or siRNA.

According to the invention, numerous vector systems allow the use of miRNAs for subsequent stable and regulated transcription of the corresponding VGLUT-siRNAs. The transcription of the miRNAs may be controlled by polymerase III promoters (for example HI or U6 promoters) and also by polymerase II promoters (Brummelkamp et al., 2002; Lee et al., 2002; Miyagishi and Taira, 2002). The sense and antisense strands of various promoters may be read off and accumulate in the cell to form 19-nt duplices with 4-nt overhangs (B) (Lee et al., 2002), or the expression of hairpin structures is used (Brummelkamp et al., 2002).

Viral vectors, for example retroviral or adenovirus-derived vectors, are preferably used for these vector systems. Viral vectors have very efficient targeted transduction of specific cells, including primary cells, and can therefore be used widely, for example, in pain therapy.

According to a further particularly advantageous embodiment, it is provided that the dsRNA is bound to at least one capsid protein which originates from a virus or is derived therefrom or from a synthetically produced viral capsid protein, associated therewith or surrounded thereby. The capsid protein may be derived from the polyoma virus. It may therefore be, for example, the virus protein 1 (VP1) and/or the virus protein 2 (VP2) of the polyoma virus. The use of such capsid proteins is known, for example, from DE 19618797 A1. The aforementioned features substantially simplify introduction of the dsRNA in to the cell.

In a preferred embodiment, the dsRNA according to the invention is expressed in that the first template (sense dsRNA) and the second template (antisense dsRNA) are under the control of two identical or different promoters. Expression takes place in vivo and is brought into the cells by vectors in the course of gene therapy.

The present invention further relates to a pharmaceutical composition containing at least one dsRNA according to the invention and/or a cell containing it, and optionally auxiliaries and/or additives.

Pharmaceutical composition: a substance corresponding to the definition in Article 1 §2 of the German law regulating the circulation of pharmaceutical compositions (AMG). In other words, substances or preparations of substances which are intended, by application onto or into the body of a human or animal,
1. to heal, relieve, prevent or detect diseases, illness, physical disorders or disease ailments,
2. to reveal the nature, the state or the function of the body or mental states,
3. to replace effective substances or body fluids produced by the human body,
4. to repel, eliminate or render harmless pathogens, parasites or extraneous substances, or
5. to influence the nature, the state or the functions of the body or mental states.

The pharmaceutical compositions according to the invention may be administered as liquid pharmaceutical preparations in the form of injection solutions, drops or syrups, as semi-solid pharmaceutical preparations in the form of granules, tablets, pellets, patches, capsules, plasters or aerosols and contain, in addition to the at least one subject of the invention, optionally excipients, fillers, solvents, diluents, dyes and/or binders, depending on the galenical form. The choice of auxiliary agents and the quantities thereof to be used depend on whether the pharmaceutical preparation is to be applied orally, stemerally, intravenously, intraperitoneally, intradermally, intramuscularly, intranasally, buccally, rectally or topically, for example to infections of the skin, the mucous membranes or the eyes. Preparations in the form of tablets, dragees, capsules, granules, drops and syrups are suitable for oral application, solutions, suspensions, easily reconstitutable dry preparations and sprays are suitable for stemeral, topical and inhalative applications. Subjects according to the invention in a deposit, in dissolved form or in a plaster, optionally with the addition of agents to promote skin penetration, are suitable percutaneous application preparations. Orally or percutaneously applicable preparation forms can release the compounds according to the invention after a delay. The amount of active ingredient to be administered to the patient varies according to the weight of patient, the method of application, the indication and the severity of the disease. 2 to 500 mg/kg of at least one subject according to the invention are usually applied. If the pharmaceutical composition is to be used, in particular, for gene therapy, a physiological sodium chloride solution, stabilizers, proteinase, DNAse inhibitors etc., are recommended as suitable auxiliaries or additives.

The present invention further relates to host cells, except for human germ cells, and human embryonic stem cells, which are transformed by at least one dsRNA according to the invention. dsRNA molecules according to the invention may be introduced into the respective host cell by conventional methods, for example transformation, transfection, transduction, electroporation or particle gun. During transformation, at least two dsRNAs which are different from one another are introduced into the cell, one strand of each dsRNA being complementary, at least in certain fragments, with the (m)RNA of a member of the VGLUT family, in particular complementary to the (m)RNA of VGLUT1, VGLUT2 or VGLUT3. The region of the dsRNA complementary with the (m)RNA of VGLUT1, 2 or 3 contains less than 25 successive nucleotide pairs.

Suitable host cells include any cells of a prokaryotic or eukaryotic nature, for example of bacteria, fungi, yeasts, vegetable or animal cells. Preferred host cells include bacterial cells such as Escherichia coli, Streptomyces, Bacillus or Pseudomonas, eukaryotic microorganisms such as Aspergillus or Saccharomyces cerevisiae or conventional baker's yeast (Stinchcomb et al. (1997) Nature 282: 39)

In a preferred embodiment, however, cells from multicellular organisms are selected for transformation by means of dsRNA constructs according to the invention. In principle, any higher eukaryotic cell culture is available as a host cell, although cells of mammals, for example monkeys, rats, hamsters, mice or humans, are quite particularly preferred. A large number of established cell lines is known to the person skilled in the art. The following cell lines are mentioned in a list, which is not exhaustive: 293T (embryonic renal cell line) (Grahan et al., J. Gen. Virol. 36:59 (1997), BHK (baby hamster renal cells), CHO (cells from hamster ovaries, Urlaub and Chasin, Proc. Natl. Accad. Sci. USA 77: 4216, (1980)), Hela (human carcinoma cells) and further cell lines—established in particular for laboratory use-, for example HEK293, SF9 or COS cells, wt-PC12 and DRG primary cultures. Quite particularly preferred are human cells, in particular neuronal stem cells and cells from the pain pathway, preferably primary sensory neurons. Human cells, in particular autologous cells from a patient, are suitable, after (in particular ex vivo) transformation with dsRNA molecules according to the invention, in other words after cell removal, optionally ex vivo expansion, transformation, selection and final retransplantation in the patient, quite particularly as pharmaceutical compositions for, for example, gene therapy.

A further preferred subject is also the use of at least one dsRNA according to the invention or pharmaceutical composition and/or of at least one cell according to the invention for producing a pharmaceutical preparation or pain remedy for the treatment of pain, in particular chronic pain, tactile allodynia, thermally triggered pain and/or inflammatory pain.

The subjects of the invention are suitable as pharmaceutical compositions, for example for nociception inhibition, for example by reducing the expression of at least one member of the VGLUT family, for example VGLUT1, -2 or -3, using dsRNA according to the invention.

Also disclosed is the use of at least one dsRNA according to the invention containing dsRNA according to the invention and/or a cell according to the invention for producing a pharmaceutical composition for the treatment of urinary incontinence; also of neurogenic bladder symptoms, pruritus, tumours, inflammation; in particular of VGLUT-associated inflammation with symptoms such as asthma; and of any disease symptoms associated with VGLUT family members.

The invention further relates to a process for the treatment, in particular pain treatment, of a non-human mammal or human, which requires the treatment of pain, in particular chronic pain, by administration of a pharmaceutical composition according to the invention, in particular those containing a dsRNA according to the invention. The invention further relates to corresponding processes for the treatment of pruritus and/or urinary incontinence.

A further preferred subject is also the use of at least one dsRNA according to the invention, in particular siRNA, and/or of a cell according to the invention for gene therapy, preferably in vivo or in vitro gene therapy. Gene therapy is understood to be a form of therapy during which, for example, an effector gene, usually a protein, and, in the present case, in particular dsRNA according to the invention, is expressed by the introduction of nucleic acids in cells.

A basic distinction is made between in vivo and in vitro processes. In the case of in vitro processes cells are removed from the organism and are transfected ex vivo with vectors and are then introduced back into the same organism or into another organism. With in vivo gene therapy, vectors, for example for combating tumours, are applied systemically (for example via the bloodstream) or directly into the tumour. According to a preferred embodiment, a vector is administered, which contains both the transcription element for the sense-dsRNA and the transcription element for the antisense-dsRNA under the control of suitable promoters. According to a further preferred embodiment, the two transcription elements may be located on different vectors.

A further preferred subject is also a diagnostic reagent containing at least one dsRNA and/or a cell according to the invention and optionally suitable additives. A diagnostic reagent herein denotes a compound or a process which may be used to diagnose a disease.

According to the invention, a further preferred subject is also a process for identifying pain-modulating substances. In a preferably upstream process step (a), over-expression of VGLUT, preferably VGLUT1, VGLUT2 or VGLUT3, takes place in a test cell. This over-expression in a test cell ensures that there is an increased concentration of VGLUT in these manipulated test cells, which are used for further examination, so the efficiency of potentially pain-modulating substances may be determined more accurately by means of scale expansion. However, in principle, cells that have not been manipulated in this manner, but nevertheless natively express VGLUT, may be used for the process according to the invention.

The preferably cultivatable cells, which may have been obtained by the placement upstream of process step (a), are subjected to the (in particular simultaneous) process steps (b) and (b′), namely (b) preferably genetic manipulation of at least one cell (test cell) with at least one dsRNA according to the invention and (b′) an (in particular simultaneous) comparative test (control test) with at least one identical cell (control cell). A comparative test of this type according to process step (b′) may follow different target directions, depending on the desired knowledge to be obtained. Various embodiments are therefore conceivable. The comparative test may thus, for example, be conducted with test cells that, in contrast to process step (b), are used without any genetic manipulation with dsRNA. Alternatively, however, control cells may also comprise an altered dsRNA, one not according to the invention, for example, or else be manipulated with a specific dsRNA that has a known effect on the VGLUT expression. Finally, process step (b′) may optionally also be omitted. In a process step (c), the test cells, which both express VGLUT and also, according to process step (b), comprise the substance to be tested, are incubated under suitable conditions. The test cells from process step (b) and the control cells according to process step (b′) are typically incubated simultaneously.

In a process step (d), for example, the binding of the test substance on the VGLUT-(m)RNA synthesized by the cells is then measured, preferably under suitable conditions. A preparation of the test cells manipulated with the test substance may, for example, be required for this purpose. Measurement of at least one of the functional parameters altered by the binding of the test substance, typically dsRNA, on the VGLUT-(m)RNA, for example, is, however, preferred. This altered parameter may, for example, be a quantifiable phenotype of the incubated cell that is adjusted by means of the binding of the test substance on VGLUT-(m)RNA, for example on the basis of the expression of the VGLUT protein suppressed by the binding. The measurement may also take place via immunofluorescence methods, for example, by means of which the concentration of VGLUT in the target cells is determined. However, the VGLUT that is over-expressed in the test cell by means of a process step (a) may (additionally) be configured with a reporter function. A fluorescence property connected to the over-expressed VGLUT by means of a corresponding gene construct (or the optional suppression of said property by means of the addition of a positively tested test substance according to the invention) would, for example, be directly measurable in the cell. Potentially pain-modulating substances are then identified, for example, via the extent of the difference between the measured value in the test cell and the measured value in the control cell, in a process step (e).

The dsRNA that is transferred into the test cells according to process step (b) or (b′) in the form of genetic manipulation, as a typical test substance of a process according to the invention, may also be transferred into the test cells via any alternative route. For example, the addition to the test cells may take place exogenously, optionally in conjunction with further chemical or physical measures known from the prior art, in order to ensure the absorption of the dsRNA into the cells, for example by means of electroporation, etc. Insofar as the dsRNA test substances applied exogenously to the test cells are unable per se to penetrate cellular membrane, their cellular membrane penetration capacity may also be increased by means of corresponding formulations, for example in liposomes or by coupling of known membrane penetration reinforcing agents, for example suitable polymers or transfection reagents.

The term pain-modulating refers to a potential regulating influence on the physiological occurrence of pain, in particular to an analgesic effect. The term substance covers any compound that is suitable as a pharmaceutical active ingredient, in particular therefore low-molecular active ingredients, but also others such as nucleic acids, fats, sugars, peptides or proteins such as antibodies. Incubation under suitable conditions herein means that the substance to be investigated can react with the cell or the corresponding preparation in an aqueous medium a defined time before measurement. The temperature of the aqueous medium may be controlled, for example at between 4° C. and 40° C., preferably at ambient temperature or at 37° C. The incubation time may be varied between a few seconds and a plurality of hours, depending on the interaction of the substance with the protein. However, times between 1 min and 60 min are preferred. The aqueous medium may contain suitable salts and/or buffer systems, so, for example, a pH between 6 and 8, preferably pH 7.0-7.5 prevails in the medium during incubation. Further suitable substances such as coenzymes, nutrients, etc. may be added to the medium. A person skilled in the art can easily determine suitable conditions as a function of the interaction of the substance to be investigated with the protein, on the basis of his experience, the literature or a few simple preliminary tests, in order thereby to obtain a measured value that is as clear as possible. A cell that has synthesized a protein is a cell which has already expressed this protein endogenously or a cell which has been genetically modified so it expresses this protein and accordingly contains the protein from the beginning of the process according to the invention. The cells may be cells from possibly immortalized cell lines or native cells originating from tissues and isolated from them, the cell assembly usually being dissolved. The preparation from these cells comprises, in particular, homogenates from the cells, the cytosol, a membrane fraction of the cells with membrane fragments, a suspension of isolated cell organelles, etc.

The criterion by which the process allows the discovery of useful substances is either the binding to the protein, which may be demonstrated, for example, by displacement of a known ligand or the extent of bound substance, or the alteration of a functional parameter by the interaction of the substance with the protein. This interaction may reside, in particular, in regulation, inhibition and/or activation of receptors, ion channels and/or enzymes. Altered functional parameters may be, for example, gene expression, ion milieu, the pH or the membrane potential, and the alteration of enzyme activity or the concentration of the second messenger. In the foregoing:
genetically manipulated refers to manipulation of cells, tissues or organisms in such a way that genetic material is introduced here;
endogenously expressed means expression of a protein comprising a cell line under suitable culture conditions, without this corresponding protein being caused to perform expression by genetic manipulation.

A further preferred embodiment of this process provides that the cell is genetically manipulated before process steps (b) and (b′).

A further preferred embodiment of this process provides that genetic manipulation allows the measurement of at least one of the functional parameters altered by the test substance.

A further preferred embodiment of this process provides that a form of a member of the VGLUT family, preferably VGLUT1, VGLUT2 or VGLUT3, which is not endogenously expressed in the cell, is expressed or a reporter gene is introduced by genetic manipulation.

A further preferred embodiment of this process provides that the bond is measured via the displacement of a known marked ligand of a member of the VGLUT family, preferably VGLUT1, VGLUT2 or VGLUT3.

A further preferred embodiment of this process provides that ≧8 hours, preferably ≧12 hours, in particular ≧24 hours elapse between the simultaneous process steps (b) and (b′) and process step (c).

The subjects according to the invention may be introduced into the cell in the above-described manner.

### BRIEF DESCRIPTION OF THE DRAWINGS

The invention will be described in further detail hereinafter with reference to the accompanying drawing figures, in which:

FIG. 1 shows strategies for RNA interference technology. Synthetic siRNA duplices may be transfected directly in cells, where they induce target mRNA degradation via the cellular RNAi machinery. On the other hand, vector-coded siRNAs are formed as hairpin-shaped precursors in the cell nucleus and are processed to siRNA in the cytoplasm of dicer.

FIG. 2a shows siRNAs which have been produced in vitro: there are basically a plurality of ways of utilising RNAi technology: chemically synthesized siRNA may be used (see FIG. 2A) or also methods from molecular biology (for example FIG. 2B).

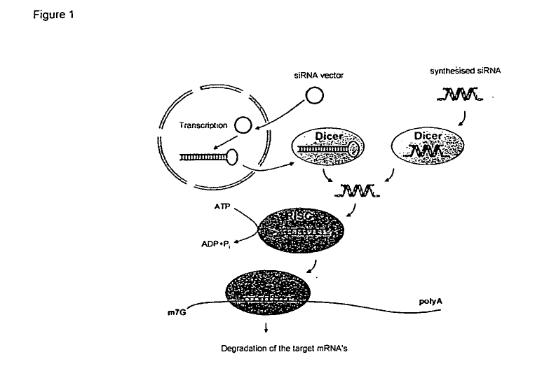

(A) Chemically synthesized siRNA bypasses the dicing step, incorporates into the RISC and leads to sequence-specific mRNA degradation. (B) Long dsRNA is processed into active siRNAs by dicers. (C) Duplex hairpin RNA may be processed into active siRNAs by dicers. (D) Incomplete duplex hairpin RNA is processed into miRNAs by dicers, incorporated into miRNPs and leads to translation inhibition. Long dsRNA molecules (B), transfected in cells, are processed in short 19 to 21 bp siRNA molecules which lead to the degradation of complementary mRNA sequences. Chemically synthesizable single-stranded 21-mers imitate the siRNAs found in vivo and, after duplex formation, are used for relatively short transient RNAi effects in vitro and in vivo (Elbashir et al., 2001a; Holen et al., 2002; Yu et al., 2002). Four embodiments of dsRNA according to the invention against VGLUT family members are therefore described.

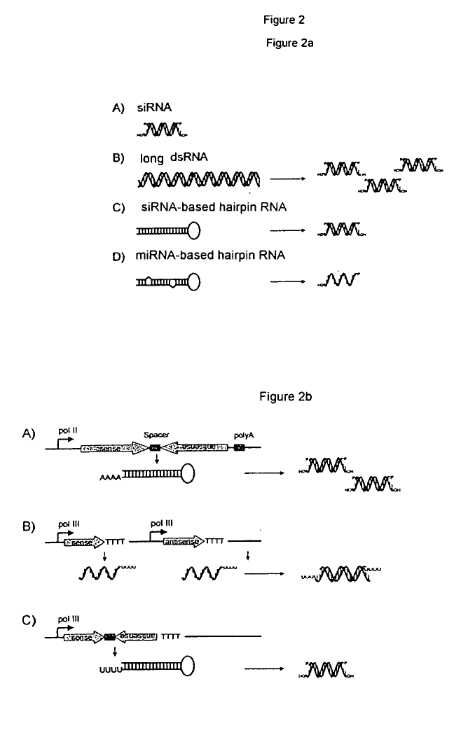

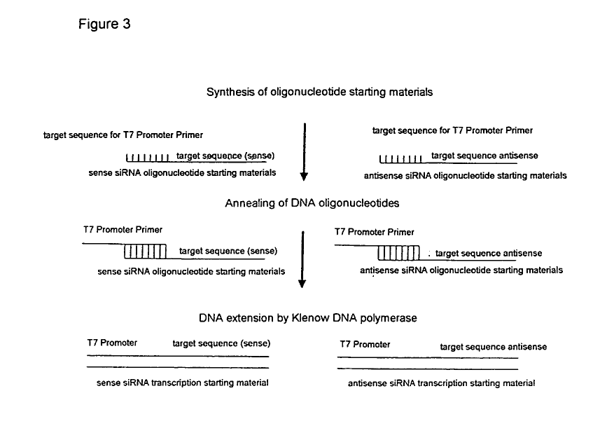

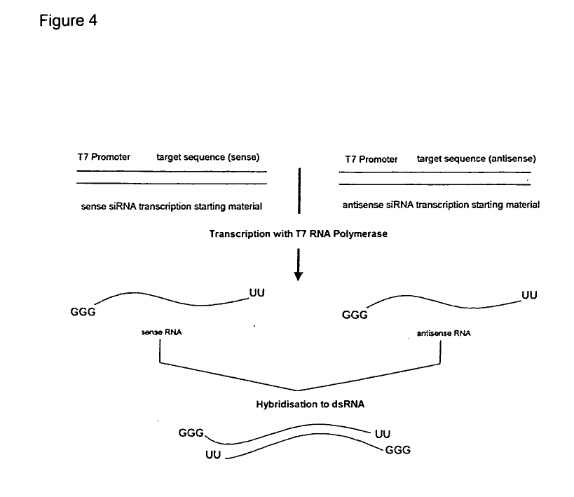

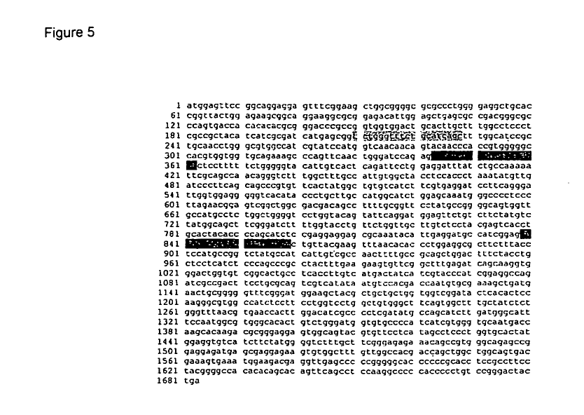

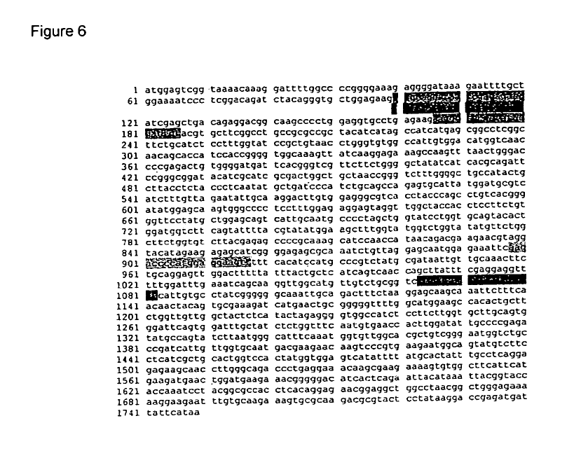

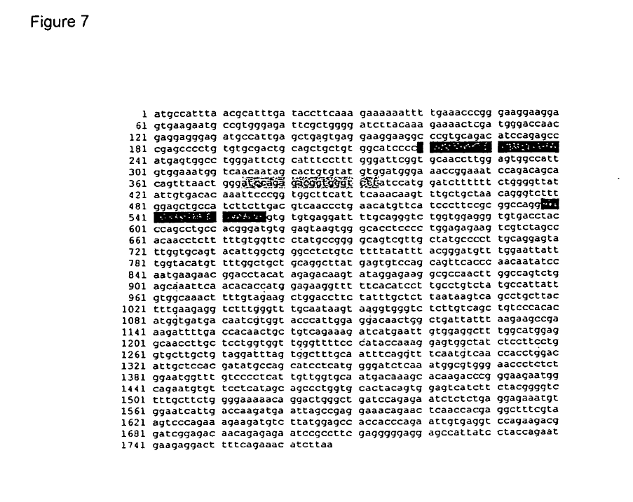

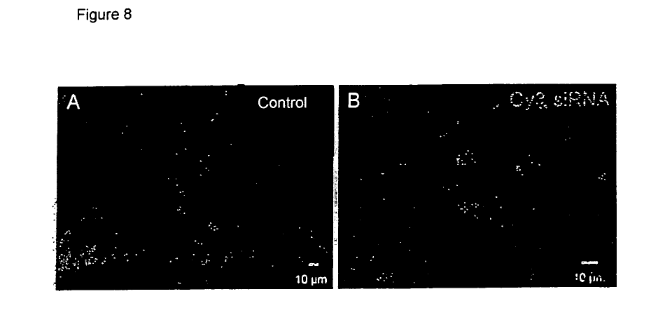

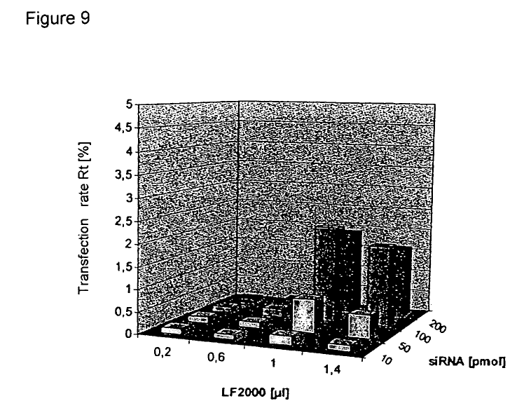

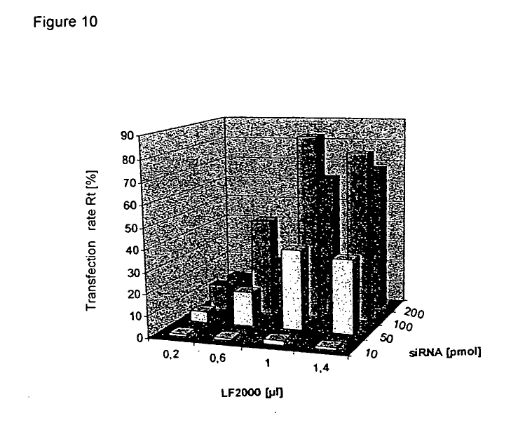

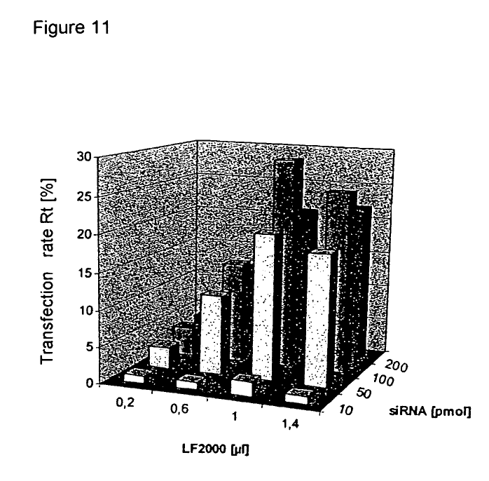

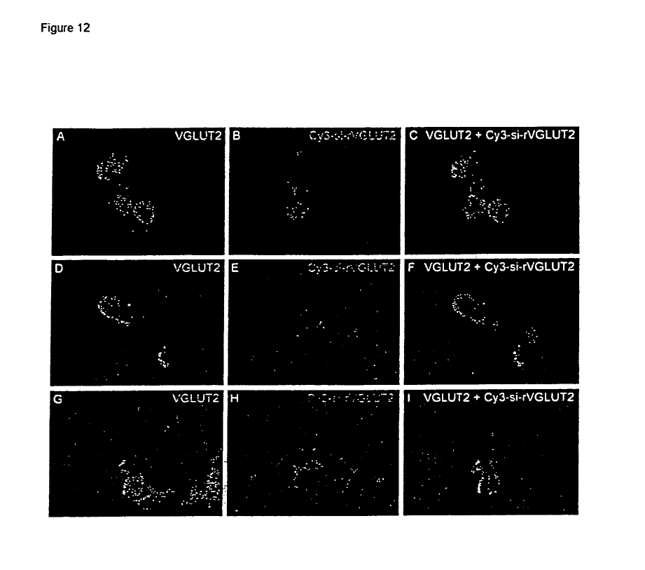

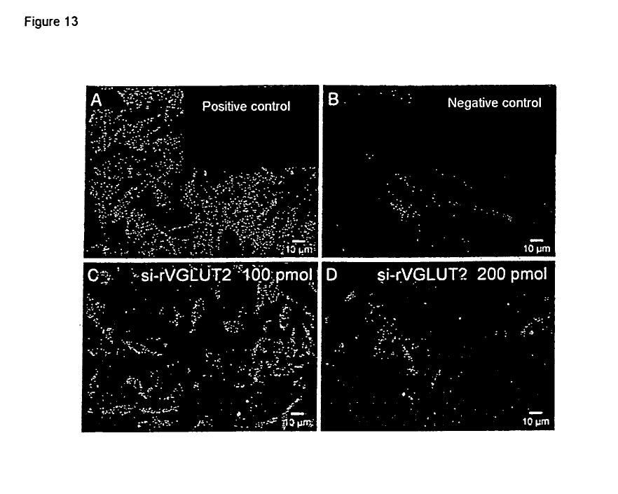

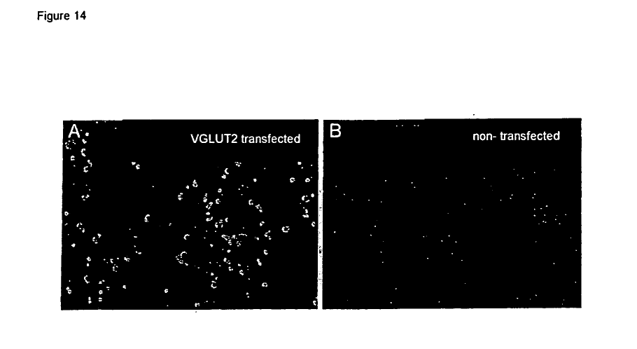

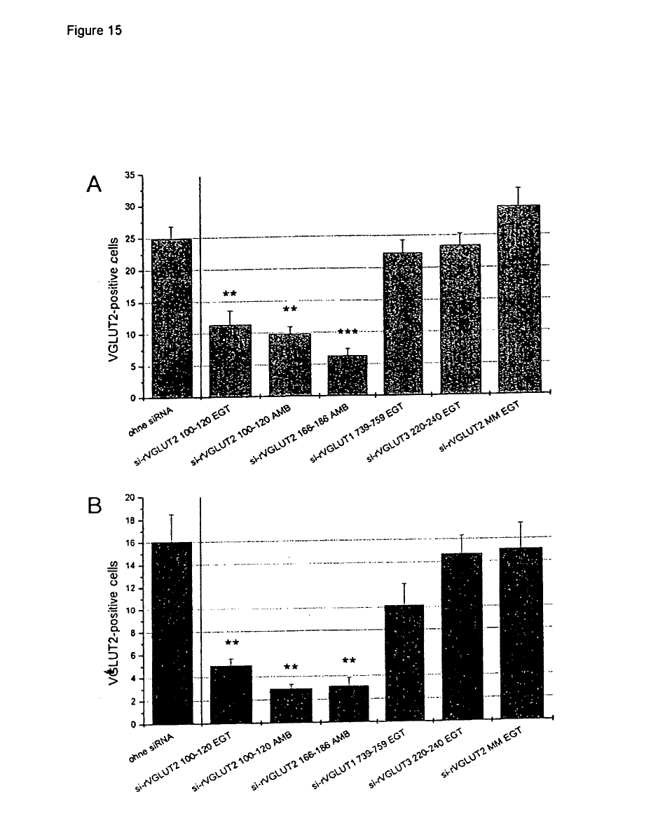

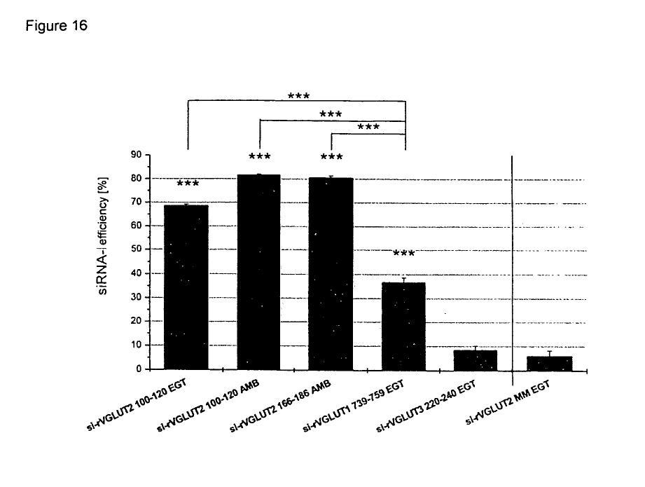

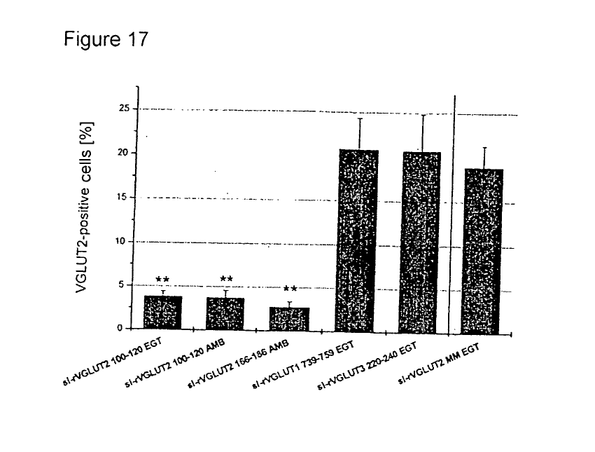

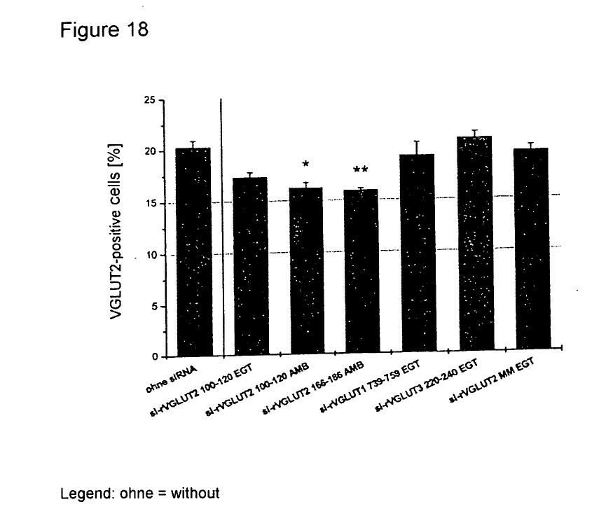

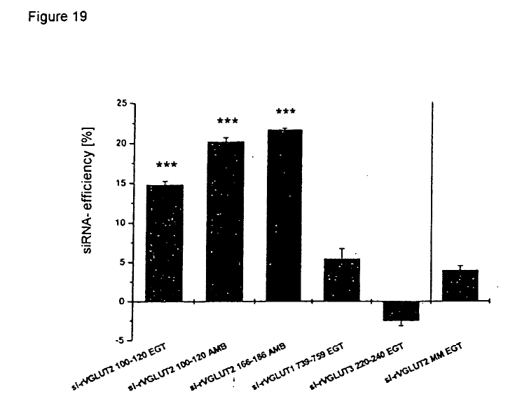

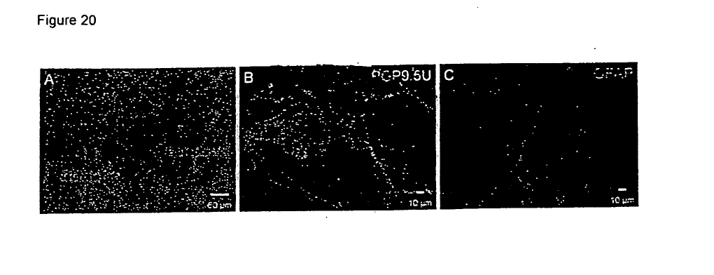

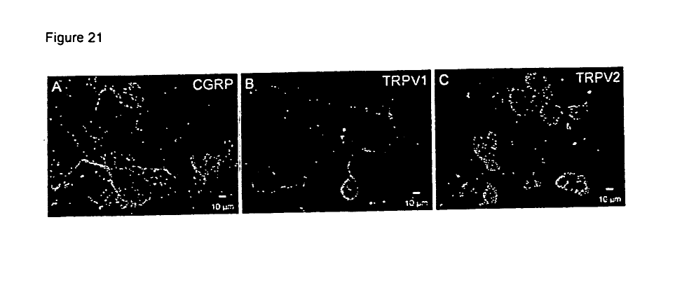

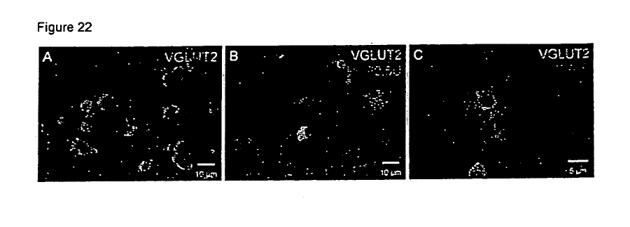

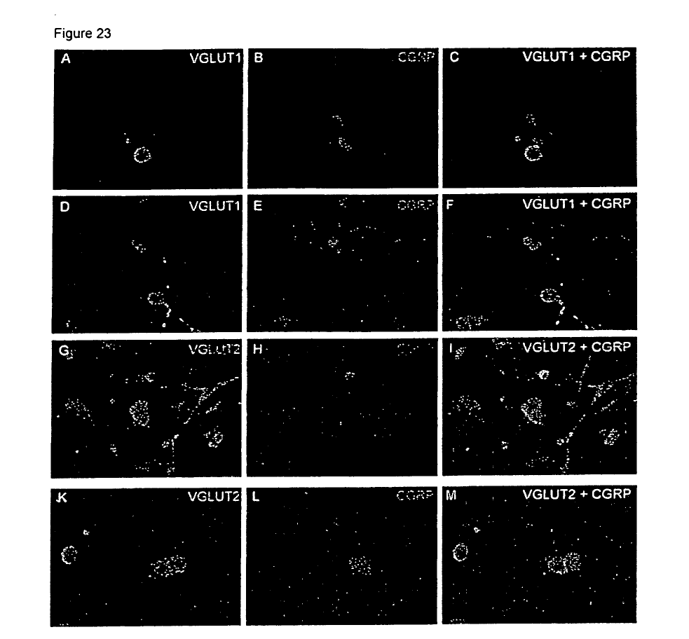

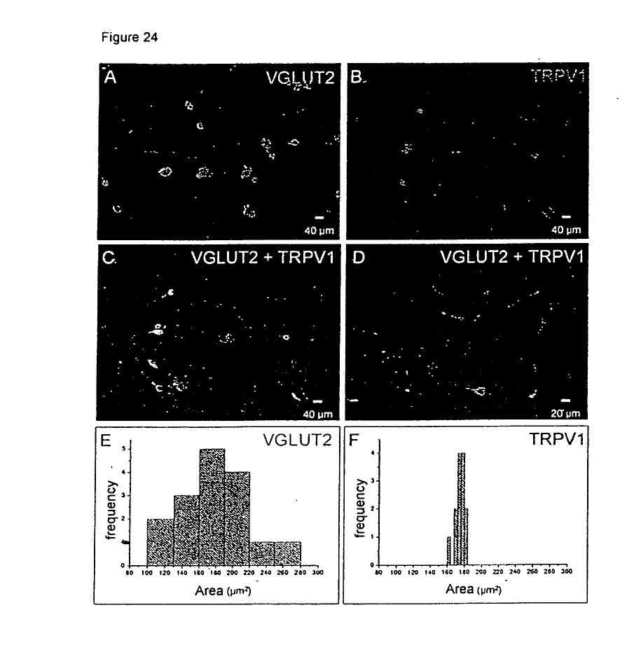

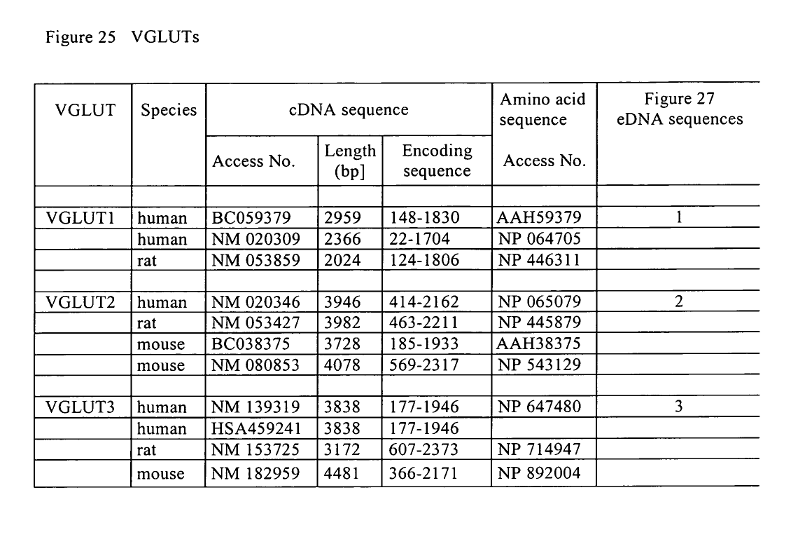

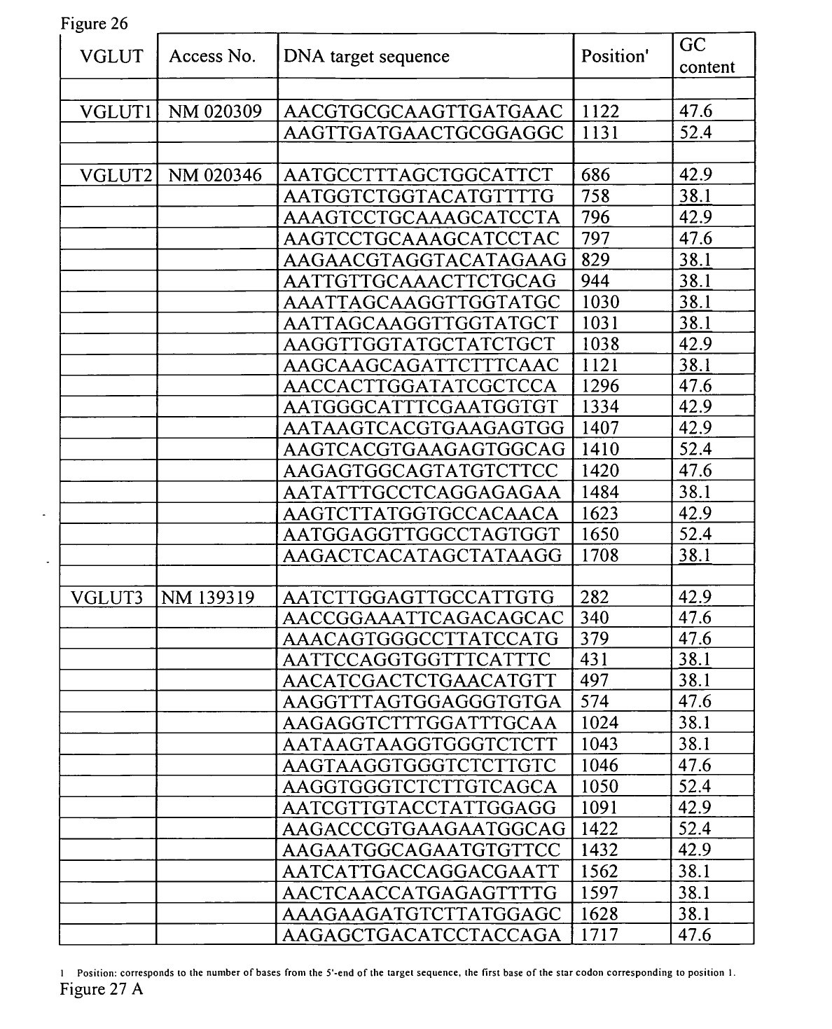

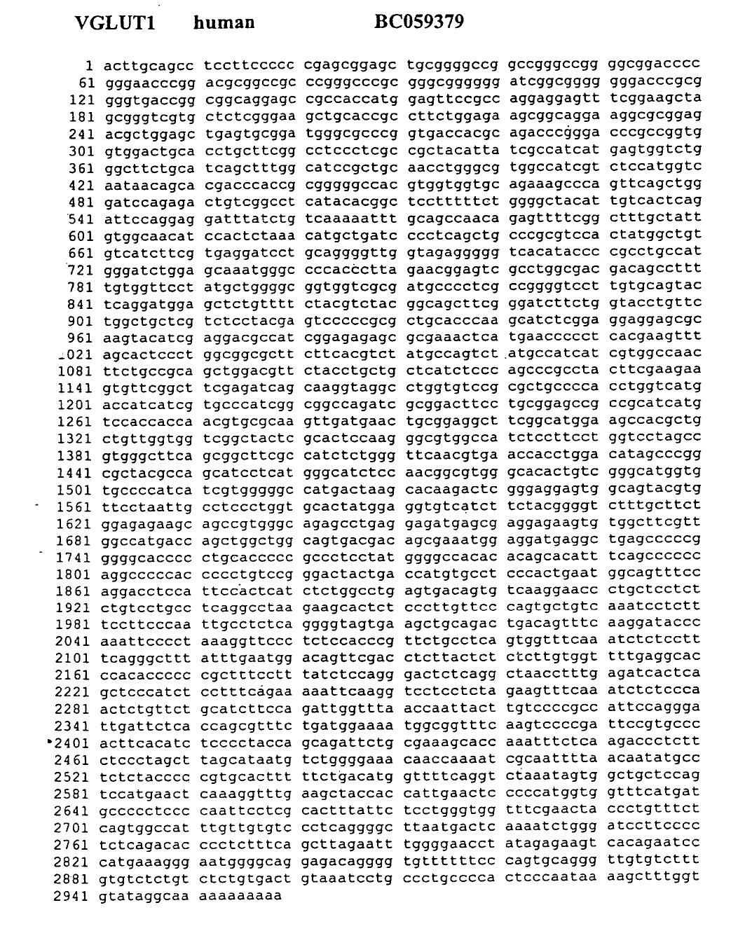

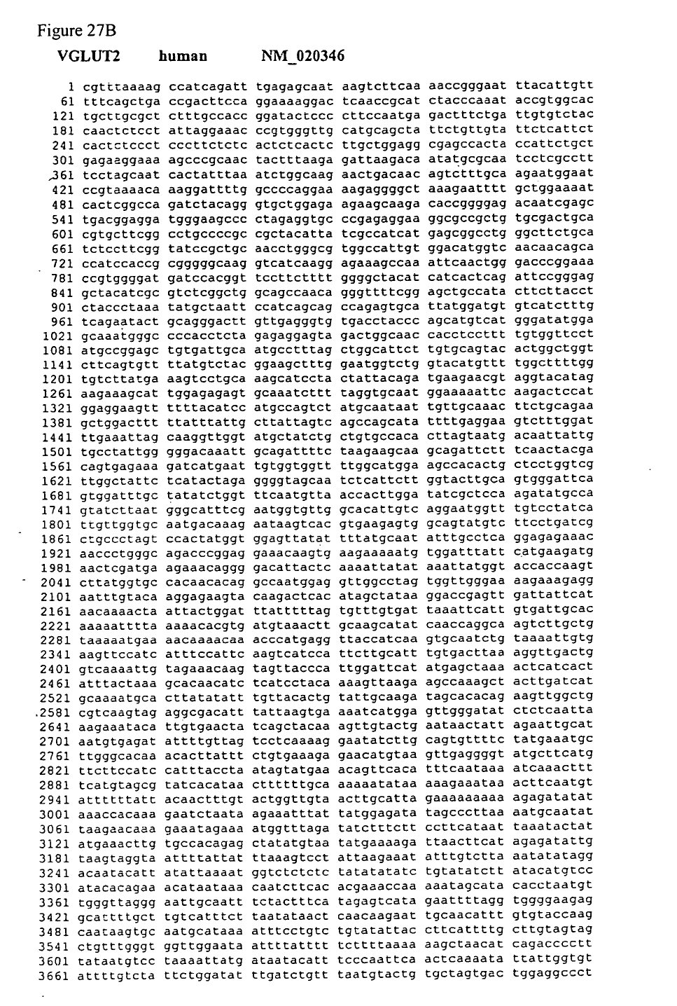

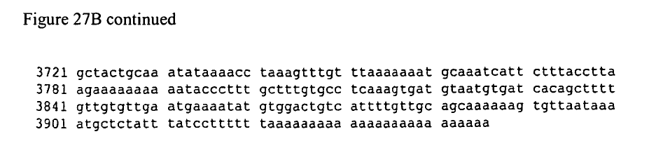

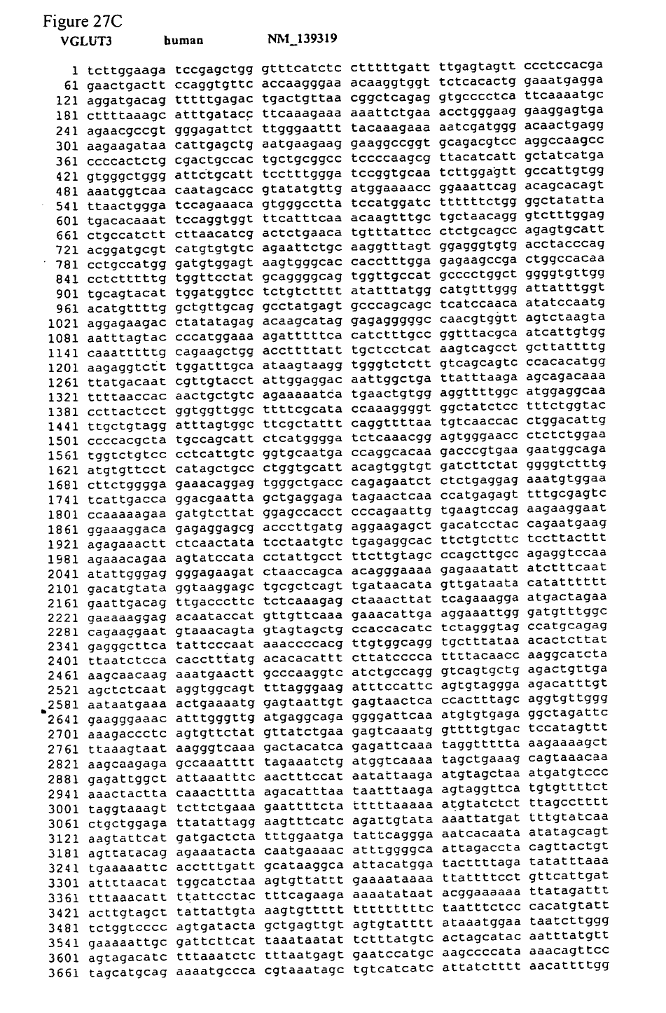

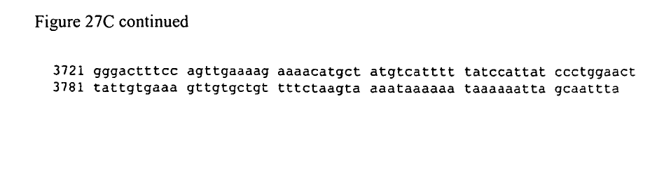

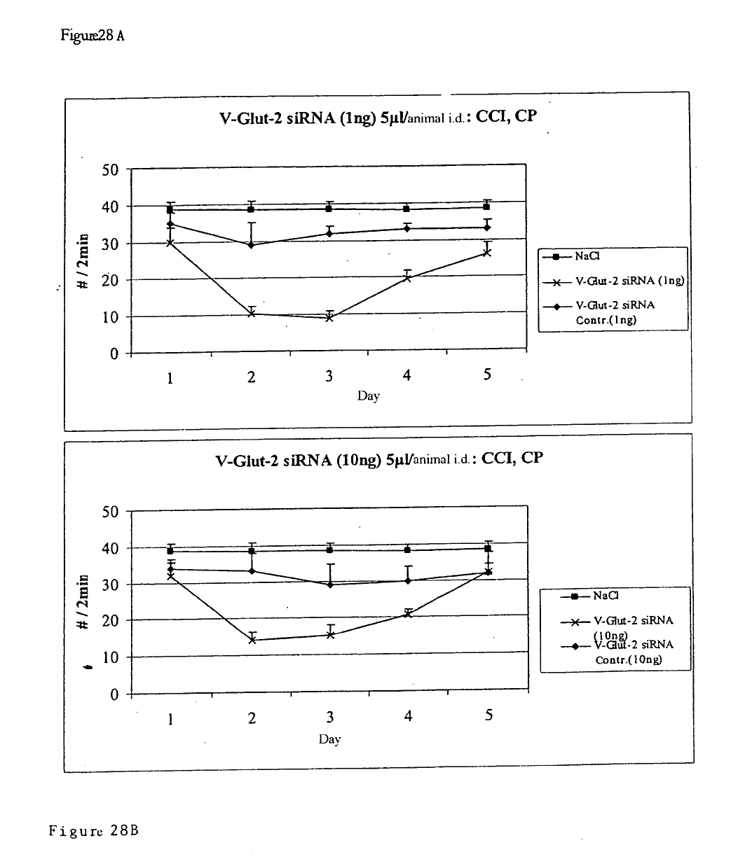

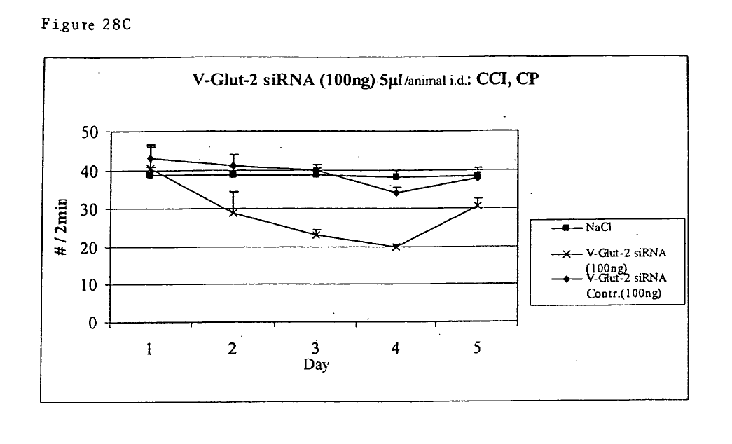

Plasmids which express dsRNA according to the invention, (in particular siRNA) (FIG. 2b), in other words short RNA fragments generated in vivo, offer a further possibility:

(A) Long hairpin RNA expressed by RNA polymerase II leads, after dicer processing, to a plurality of siRNAs with a wide variety of sequence specificities.

(B) Tandem pol III promoters allow the expression of individual sense and antisense strands which accumulate in the cell to active siRNAs.

(C) An individual pol III promoter allows expression of a short hairpin-shaped (sh)RNA which is processed to active siRNA by dicers.

Using RNA polymerase III promoters such as U6 or H1, it is possible to express dsRNA and then siRNA molecules intracellularly and therefore to establish stable RNAi systems in mammalian cells (Brummelkamp et al., 2002; Lee et al., 2002; Miyagishi and Taira, 2002). Either the sense or antisense strands of various promoters may be read off and accumulate in the cell to 19-nt duplices with 4-nt overhangs, or the expression of hairpin structures is utilized. In both cases, effective, stable suppression of gene expression is achieved by the RISC-mediated RNAi process. Although the small size of a transcript which may be expressed by the pol III promoter does not initially impede siRNA technology, it restricts the number of different siRNAs which may be formed by a transcript (Myslinski, 2001).

FIG. 3 shows the production of the DNA patterns for siRNA synthesis.

FIG. 4 shows the transcription and hybridization of the siRNA.

FIG. 5 shows VGLUT1 cDNA with siRNA target sequence; Gene Bank Accession No. U07609. Highlighted in color: initiating codon (yellow), siRNA si-rVGLUT1 739-759 EGT (red), primer rVGLUT1 (2_{—}4)F (light grey), primer rVGLUT1 (2_{—}4)R (dark grey).

FIG. 6 shows VGLUT2 cDNA with siRNA target sequences; Gene Bank Accession No. NM_{—}053427. Highlighted in color: initiating codon (yellow), siRNA si-rVGLUT2 100-120 EGT (red), siRNA si-rVGLUT2 100-120 AMB (green), siRNA si-rVGLUT2 166-186 AMB (blue), primer rVGLUT2(8_{—}9)F (light grey), primer rVGLUT2(8_{—}9)R (dark grey).

FIG. 7 shows VGLUT3 cDNA with siRNA target sequence; Gene Bank Accession No. AJ491795. Highlighted in color: initiating codon (yellow), siRNA si-rVGLUT3 220-240 EGT (red), primer rVGLUT3(4_{—}5)F (light grey), primer rVGLUT3(4_{—}5)R (dark grey).

FIG. 8 shows the transfection of PC12 MR_A cells with Cy3-labelled siRNA; Cy3-labelled siRNA (100 pmol) was transfected using LF2000 (1 μl) in cells of the PC12 MR-A cell line (B). LF2000 was dispensed with during the control (A). The cells were fixed for 24 hours after transfection and the cell nuclei complementarily colored with DAPI.

FIG. 9 shows the transfection rate in DRG primary cultures. The transfection rate in 250,000 cells of a DRG primary culture (P0, 1 d.i.v.) was determined in a 24-well plate using various amounts of Lipofectamine™ 2000 (0.2-1.4 μl) and different Cy3-siRNA concentrations (10 to 200 pmol) 24 hours after transfection using a fluorescence microscope.

FIG. 10 shows the transfection rate in PC12 MR-A cells. The transfection rate in 250,000 PC12 MR-A cells was determined in a 24-well plate using various amounts of LF 2000 (0.2-1.4 μl) and different Cy3-siRNA concentrations (10 to 200 pmol) 24 hours after transfection using a fluorescence microscope.

FIG. 11 shows the transfection rate in wt-PC12 cells. For this purpose, the transfection rate in 250,000 wt-PC12 cells was determined in a 24-hole plate using various amounts of LF 2000 (0.2-1.4 μl) and different Cy3-siRNA concentrations (10 to 200 pmol) 24 hours after transfection using a fluorescence microscope.

FIG. 12 shows the results of the treatment of a DRG primary culture with Cy3-labelled siRNA against VGLUT2. The suppression of VGLUT2 protein expression by means of Cy3-labelled siRNA in neurons of a DRG primary cultre (P0, 2 d.i.v.) was determined by immune fluorescence 48 hours after transfection with LF2000 (1 μl/well) in a 24-well plate. VGLUT2 (guinea pig, 1:800, green), Cy3-si-rVGLUT2 166-186 AMB (100 pmol, red) and superimposed depiction of the VGLUT2 and Cy3-siRNA signals with DAPI-colored cell nuclei (C, F, I).

FIG. 13 shows VGLUT2-protein expression in PC12 MR-A cells and protein suppression by means of siRNA. Immunocytochemical detection of VGLUT2 protein expression was carried out (A) in cells of cell line PC12 MR-A and reduction of the VGLUT2 protein level by means of siRNA against VGLUT2 (si-rVGLUT2 100-120 EGT), 100 pmol siRNA (C), 200 pmol siRNA (D) and negative control (B) without primary antibodies against VGLUT2 (rabbit, 1:800).

FIG. 14 shows the detection of VGLUT2 in transfected wt-PC12 cells. VGLUT2 protein with primary antibodies against VGLUT2 (rabbit, 1:800) was detected by immunocytochemistry 24 hours after transfection of rVGLUT2 plasmids by means of LF2000™ in wt-PC12 cells: (A) VGLUT2-positive cells (A488-labelled, green) in rVGLUT2-transfected cells; (B) no VGLUT2-immune reactivity in non-transfected cells (negative control).

FIG. 15 shows the siRNA treatment of VGLUT2-cotransfected wt-PC 12 cells. The immunocytochemically labelled, VGLUT2-positive wt-PC12 cells were counted out 24 hours after co-transfection of rVGLUT2 plasmid with siRNAs against VGLUT2 (si-rVGLUT2 100-120 EGT, si-rVGLUT2 100-120 AMB, si-rVGLUT2 166-186), against VGLUT1 (si-rVGLUT1 739-759 EGT) and against VGLUT3 (si-rVGLUT3 220-240 EGT), and with a mismatch siRNA (si-rVGLUT2 MM EGT). Illustration of the mean values±S.E.M. for n=6 per group. *p<0.05; **p<0.01 and ***p<0.001 in comparison with the control group without siRNA treatment (ANOVA, Bonferroni Test). Comparison of two methods of evaluation: (A) manual counting (B) digital counting.

FIG. 16 shows the efficiency of the siRNAs in wt-PC12 cells. The siRNA efficiencies (percentage reduction in VGLUT2-positive cells based on VGLUT2 expression without siRNA) were compared 24 hours after co-transfection of rVGLUT2 plasmid and siRNA in wt-PC12 cells. Illustration of the mean values±S.E.M. for n=6 per group. ***p<0.001 in comparison with the treatment with mismatch siRNA (ANOVA, Bonferroni test).

FIG. 17 shows the siRNA treatment of wt-PC12 cells 6 hours before transfection with rVGLUT2. Immunocytochemically labelled VGLUT2-positive wt-PC12 cells were counted out 24 hours after treatment with siRNAs against VGLUT2 (si-rVGLUT2 100-120 EGT, si-rVGLUT2 100-120 AMB, si-rVGLUT2 166-186), against VGLUT1 (si-rVGLUT1 739-759 EGT) and against VGLUT3 (si-rVGLUT3 220-240 EGT), and with a mismatch siRNA (si-rVGLUT2 MM EGT). The cells were transfected 6 hours after siRNA treatment with rVGLUT2 plasmid. Illustration of the mean values±S.E.M. for n=6 per group. **p<0.01 in comparison with the treatment with mismatch siRNA (ANOVA, Bonferroni test).

FIG. 18 shows the influence of siRNA treatment 24 hours after rVGLUT2 transfection. Immunocytochemically labelled VGLUT2-positive wt-PC12 cells were counted out 24 hours after treatment with siRNAs against VGLUT2 (si-rVGLUT2 100-120 EGT, si-rVGLUT2 100-120 AMB, si-rVGLUT2 166-186), against VGLUT1 (si-rVGLUT1 739-759 EGT) and against VGLUT3 (si-rVGLUT3 220-240 EGT), and with a mismatch siRNA (si-rVGLUT2 MM EGT). The cells were transfected 24 hours before siRNA treatment with rVGLUT2 plasmid. Illustration of the mean values±S.E.M. for n=6 per group. *p<0.05 and **p<0.01 in comparison with the treatment with untreated cells (ANOVA, Bonferroni test).

FIG. 19 shows the efficiency of the siRNAs 24 hours after rVGLUT2 transfection. The siRNA efficiencies (proportion of VGLUT2 suppression based on VGLUT2 expression without siRNA) were compared 24 hours after siRNA treatment in wt-PC12 cells, which had been transfected with rVGLUT2 plasmid 24 hours prior to the siRNA treatment. Illustration of the mean values±S.E.M. for n=6 per group. ***p<0.001 in comparison with the treatment with mismatch siRNA (ANOVA, Bonferroni test).

FIG. 20 shows the characterisation of the cells in a primary culture of the spinal ganglion. Light and fluorescence microscopic documentation on different types of cells in a primary culture of the spinal ganglion (postnatal day 2, 1 to 5 days in vitro) are illustrated: (A) light microscopic documentation (1 d.i.v.); (B, C) immunocytochemical detection of (B) neurons (5 d.i.v) by means of primary antibodies against PGP9.5U (1:1500) and of (C) Schwann cells (5 d.i.v.) by means of primary antibodies against GFAP (1:5000). The cell nuclei are complementarily colored blue in (B) and (C) with DAPI.

FIG. 21 shows nociceptive neurons in DRG primary cultures. Nociceptive label proteins were detected immunocytochemically in cells of a DRG primary culture (P², 8 d.i.v.) by means of primary antibodies against (A) CGRP (rabbit, 1:8000); (B) TRPV1 (rabbit, 1:250); (C) TRPV2 (rabbit, 1:400).

FIG. 22 shows VGLUT2 protein expression in DRG primary cultures. For this purpose, VGLUT2 protein expression in primary cultures of spinal ganglia was characterized immunocytochemically: (A) VGLUT2 (guinea pig, 1:800); (B) VGLUT2 (1:800, green), PGP9.5 (rabbit, 1:1500, red) co-expression of VGLUT2 and PGP9.5 (yellow); (C) VGLUT2 (1:800, green), GFAP (rabbit, 1:5000, red). All cell nuclei were complementarily colored with DAPI.

FIG. 23 shows the VGLUT1 and VGLUT2 expression in peptidergic DRG neurons. Double immune fluorescence of VGLUT1 and VGLUT2 with CGRP in DRG neurons (A-M) is illustrated: (A, C, D, F) VGLUT1 (guinea pig, 1:800, green); (B, C, E, F, H, I, L, M) CGRP (rabbit, 1:5000, red); (G, I, K, M) VGLUT2 (guinea pig, 1:800, green). VGLUT1 and CGRP are expressed in various subpopulations (C, F), as CGRP coexists with VGLUT2 (I, M, yellow signal).

FIG. 24 shows the expression of VGLUT2 in TRPV1-positive neurons. VGLUT2 expression in TRPV1-positive neurons of a DRG primary culture was detected immunocytochemically: (A) VGLUT2 (guinea pig, 1:800, green); (B) TRPV1 (rabbit, 1:250, red); (C) VGLUT2 co-localisation in TRPV1-positive neurons (yellow signal); (D) enlargement of part of Fig. C; signals of VGLUT2 in the cell soma (yellow) and in the axon (yellow, green) of the TRPV1-positive neuron; (E, F) frequency distribution of the cell surfaces of VGLUT2 or TRPV1-positive neurons.

FIG. 25 gives an overview of the various VGLUT sequences (human, rat) (VGLUT1, VGLUT2, VGLUT3), including the respective database accession code.

FIG. 26 shows DNA target sequences of VGLUT-isoform-specific siRNAs. The preferred sequences are shown in bold print. Homologues have been tested by the Smith-Waterman algorithm.

FIG. 27 shows the nucleotide sequences of VGLUT1, VGLUT2 and VGLUT3 (human in each case).

FIG. 28 shows the results of the in vivo tests on rats using Bennett's pain model (see embodiment 4). Three different doses were tested. FIGS. 28A, 28B and 28C show the results of the tests, using 1 ng, 10 ng and 100 ng of test substance (VGLUT2-siRNA) and corresponding amounts of control siRNA. In addition, NaCl was administered to each animal as a further control. The anti-allodynic effect of VGLUT2-siRNA (cross symbol) can be seen clearly, in particular, in FIG. 28A.

The present invention is characterized in more detail by the following practical examples.

### EXAMPLES

### Example 1

A) Design of siRNA Molecules

The design of the siRNA molecules according to the invention used corresponded to particularly preferred embodiments. FIGS. 5, 6 and 7 show the encoding sequences of the three different vesicular glutamate transporters. Colored highlighting is used for the initiating codon (yellow), and for the primer pairs used for the (quantitative) determination (light and dark grey) respectively. The target sequences of the various siRNAs are also highlighted in color (red, green, blue).

B) Production of the siRNA

The siRNAs used were ordered for synthesis by Eurogentec (EGT), on the one hand, and were self-made using the siRNA construction kit from Ambion (AMB), on the other hand. The following siRNA molecules were ordered for synthesis at Eurogentec:

| | |
|---|---|
| si-rVGLUT1 739-759 EGT | |
| 5′ AGC GCC AAG CUC AUG AAC CTT 3′ | GC content: 52.4% |
| 3′ TT UCG CGG UUC GAG UAC UUG G 5′ | |
| si-rVGLUT2 100-120 EGT (active siRNA) | |
| 5′ GCA GGA UAA CCG AGA GAC CTT 3′ | GC content: 42.8% |
| 3′ TT CGU CCU AUU GGC UCU CUG G 5′ | |
| si-rVGLUT3 220-240 EGT | |
| 5′ GCG GUA CAU CAU CGC UGU CTT 3′ | GC content: 52.4% |
| 3′ TT CGC CAU GUA GUA GCG ACA G 5′ | |
| si-rVGLUT2 MM EGT (control siRNA) | |
| 5′ GGA CUA GCA AAG CGA GCC ATT 3′ | GC content: 42.8% |
| 3′ TT CCU GAU CGU UUC GCU CGG U 5′ | |

The following siRNA molecules were produced using the Silencer™ siRNA construction kit from Ambion:

| | |
|---|---|
| si-rVGLUT2 100-120 AMB (active siRNA) | |
| 5′ GCA GGA UAA CCG AGA GAC CTT 3′ | GC content: 42.8% |
| 3′ TT CGU CCU AUU GGC UCU CUG G 5′ | |
| si-rVGLUT2 166-186 AMB | |
| 5′ GGC UCC GCU AUG CGA CUG UTT 3′ | GC content: 57.1% |
| 3′ TT CCG AGG CGA UAC GCU GAC A 5′ | |

A proportion of the self-produced siRNAs was labelled with the dye Cy3 using the Silencer siRNA labelling kit from Ambion. The aforementioned sequences were used for the in vitro experiments described hereinafter.

Production of siRNA Using Silencer siRNA Construction Kit (Ambion)

In order to produce efficient transcription patterns for siRNA synthesis, the sense and antisense oligonucleotides have to be converted in dsRNA using T7 promoter at the 5′ end. This is achieved by hybridizing the two oligonucleotides with the T7 promoter primer and lengthening them by a subsequent DNA polymerase reaction (cf. FIG. 3).

The sense and antisense siRNA templates are transcribed in separate reaction mixtures for 2 hours. The mixtures are then blended and the common reaction mixture incubated overnight. The separated transcription mixtures prevent potential competition around the transcription reagents between the templates, as this could limit the synthesis of one of the two strands of siRNA. Hybridization of the two siRNA strands is simplified by mixing the transcription mixtures and continuous RNA synthesis thus permitted, increasing the yield of dsRNA. The siRNA obtained by in vitro transcription has, at the 5′ end, overhanging leader sequences which have to be removed before transfection. This leader sequence is digested by an individual strand-specific ribonuclease. The DNA template is removed by DNase digestion in the same reaction mixture (cf. FIG. 4).

The resultant siRNA has to be cleaned up from the mixture of nucleotides, enzymes, short oligomers and salts, using RNA columns.

The siRNA purified in this way is eluted in nuclease-free water and is then available for transfection.

Procedure

A 100 μM solution of each siRNA ONV was produced from the 200 μM stock solution. The following respective reaction mixtures were produced for hybridization of the siRNA ONV with the T7 promoter primer for sense and antisense:

| | |
|---|---|
| T7 promoter primer | 2 μl |
| DNA hyb buffer | 6 μl |
| Sense/antisense siRNA ONV | 2 μl |

The mixtures were first heated to 70° C. for 5 min, then kept at ambient temperature for 5 min. The following reaction mix was then fed to the reaction mixtures, carefully mixed and incubated for 30 min at 37° C.:

| | |
|---|---|
| 10 × Klenow reaction buffer | 2 μl |
| 10 × dNTP mix | 2 μl |
| Nuclease-free water | 4 μl |
| Exo-Klenow DNA polymerase | 2 μl |

For both DNA formulations, a respective transcription reaction mixture was produced at ambient temperature in order to synthesize the sense and antisense ssRNA strands. For this purpose, the following components were combined in the specified sequence, were carefully mixed, without pipetting, and were incubated for 2 hours at 37° C.:

| | |
|---|---|
| Sense or antisense DNA template | 2 μl |
| Nuclease-free water | 4 μl |
| 2 × NTP mix | 10 μl |
| 10 × T7 reaction buffer | 2 μl |
| T7 enzyme mix | 2 μl |

After the 2 hours, two transcription mixtures were pipetted together and incubated overnight at 37° C.

The following reaction mixture was made up to digest the hybridized dsRNA with RNase and DNase and was added to the dsRNA by pipetting, carefully mixed and incubated at 37° C. for 2 hours:

| | |
|---|---|
| Digestion buffer | 6 μl |
| Nuclease-free water | 48.5 μl |
| RNase | 3 μl |

400 μl siRNA binder buffer were then fed to nuclease digestion and incubated for 2 to 5 min at ambient temperature. The filter membrane also supplied was, in the meantime, moistened with 100 μl siRNA. The siRNA was applied to the moistened filter in the siRNA binder buffer and centrifuged for 1 min at 10,000 rpm. The flow was discarded and the filter membrane washed twice with 500 μl of the siRNA washing buffer in each case and centrifuged (2 min at 10,000 rpm). The purified siRNA was then eluted in 100 μl 75° C. hot nuclease-free water and centrifuged off into a clean receiver (2 min at 12,000 rpm). The siRNA was stored at −20° C. or −80° C. until use.

Synthesis at Eurogentec and siRNA Duplex Formation

The RNA oligonucleotides synthesized by Eurogentec were brought into a 50 μM solution by means of DEPC-treated H₂O and aliquoted. 30 μl of the RNA oligonucleotide solutions belonging together were mixed in each case with 15 μl 5× annealing buffer (final concentration: 20 μM siRNA duplex; 50 mM tris pH 7.5-8.0; 100 mM NaCl in DEPC-H₂O). The solution was heated for 1 to 2 min in a water bath at 90 to 95° C. and left to cool for 45 to 60 min at ambient temperature. The siRNA was stored at −20° C. until use.

Labelling of the siRNA with Cy3

The siRNAs synthesized by Eurogentec as well as the self-produced duplex siRNAs were used for siRNA labelling with the fluorescence dye Cy3. The following reaction mixture was produced and incubated for 1 hour at 37° C. in order to label 5 μg siRNA:

| | |
|---|---|
| Nuclease-free water | 18.3 μl |
| 10 × labelling mix | 5.0 μl |
| 21-mer duplex siRNA (20 μM) | 19.2 μl |
| 3 labelling reagent | 7.5 μl |

The Cy3-labelled siRNA was purified with ethanol precipitation. For this purpose, 0.1 volume 5 M NaCl and 2.5 volumes 100% ethanol were added to the reaction mixture, thoroughly mixed and stored for 60 min at −80° C. The precipitate was pelletized by centrifugation for 20 min (>8,000×g), the supernatant being carefully removed without destroying the pellet, and was finally washed with 175 μl 70% ethanol. After centrifuging off (5 min at >8,000×g), all the supernatant was removed, the pellet dried at ambient temperature for 5 to 10 min and finally dissolved in a corresponding amount of nuclease-free water (19.2 μl in this case).

### Example 2

### Use of VGLUT siRNAs in Various in vitro Models

In order to test the efficacy of the siRNAs produced, they were used in various in vitro models and protein expression was then determined by immunocytochemistry.

A) Optimization of the Transfection Conditions

Highly efficient siRNA gene suppression necessitates not only the actual effectiveness of the siRNA but also a high transfection rate of the respective cells. The cell density, the amount of transfection reagent and the concentration of the siRNA play an important part in it. While varying these various parameters, the transfection rate (R_{T}=number of transfected cells/total number of cells) was determined using Cy3-labelled siRNA. Lipofectamine™ 2000 was used as the transfection reagent.

FIG. 8 shows examples of the results of localisation of Cy3-labelled siRNA 24 hours after transfection with LF2000™. In the PC12 MR-A cells shown here, the labelled SiRNA accumulates in the cytoplasm predominantly in the vicinity of the cell nucleus.

Trasfection of spinal ganglion cells in a primary culture proved extremely difficult. As shown in FIG. 9, a maximum transfection rate of ˜2% maximum is achieved during transfection of the cells with Cy3-labelled siRNA using LF2000. No preference for the transfection of a specific cell type was discerned, neuronal and non-neuronal cells similarly exhibiting a Cy3 fluorescence signal. However, as the maximum neuron content was 60%, the yield of siRNA-transfected neurons was very low.

The maximum transfection rates (R_{Tmax}≈80%) were achieved with cells in cell lines PC12 MR-A and PC12 MR-B, as shown, for example, in FIG. 10, for the cell line PC12 MR-A.

Normal wt-PC12 cells may also be transfected well with siRNA. FIG. 11 shows the different transfection rates for the various transfection conditions. The maximum transfection rate was ˜28%.

B) Suppression of VGLUT Expression by siRNA in Various in vitro Models

1. Suppression of Endogenous VGLUT Expression in DRG Primary Cultures

The cells of DRG primary cultures were transfected with various siRNAs directly after dissociation of the ganglia and purification of the cell suspension with the transfection reagent LF2000™. The transfected cells were either seeded in normal culture dishes or cultivated in a 24-well plate on poly-L-lysine-coated cover slips.

DRG primary cultures, which had been transfected with Cy3-labelled siRNA (Cy3-si-rVGLUT2 166-186 AMB), were fixed 48 hours after transfection and characterized immunocytochemically with respect to theirVGLUT2 protein expression. FIG. 12 shows the result of such an siRNA treatment.

Approximately 2% transfected cells, which could be detected by their Cy3 dye, were on the siRNA-treated cover slips. None of these siRNA-transfected cells showed a clear protein signal for VGLUT1 after immunocytochemical labelling of the vesicular glutamate transporterVGLUT2. On the other hand, all VGLUT2-positive cells were without signals of the Cy3-labelled siRNA.

2. Suppression of Endogenous VGLUT Expression in Established Cell Lines

The differentiated cell lines PC12 MR-A and PC12 MR-B express the vesicular glutamate transporterVGLUT1 and VGLUT2. FIG. 13 shows the expression of the VGLUT2 protein in the PC12 MR-A cells.

Under optimized conditions, these cells were transfected with siRNA against VGLUT2 (si-rVGLUT2 100-120 EGT). As shown in FIG. 13, a reduction in the VGLUT2 immune reactivity is achieved with 100 pmol siRNA (C) and most cells are without VGLUT2 immune reactivity or have only slight VGLUT2 immune reactivity at 200 pmol siRNA (D).

3. Suppression of VGLUT2 Expression in Transiently Transfected wt-PC12 Cells

Cells of the normal wt-PC12 cell line express only VGLUT1 and no VGLUT2. For this reason, these endocrine vesicle-producing cells are suitable as a model system for suppression experiments after transient VGLUT2 transfection. An EGFP vector, of which the gene product, the green fluorescent protein (GFP), may be detected directly by a fluorescence microscope was used to optimize DNA transfection. An EGFP transfection rate of ˜40% was achieved by optimising the DNA transfection conditions. A similar high transfection rate could also be achieved in the case of transfection with rVGLUT2 plasmids (FIG. 14).

The siRNA suppression experiments were configured in different ways:

Co-transfection of siRNA cDNA vector

siRNA transfection 6 hours before cDNA

cDNA transfection 24 hours before siRNA transfection.

Co-transfection was initially carried out with rVGLUT2 plasmids and various siRNAs. Three siRNAs against VGLUT2 were used, as well as siRNAs against VGLUT1, VGLUT3 and a mismatch siRNA for specificity control. The cells were fixed 24 hours after transfection and the VGLUT2 protein expression detected by immunocytochemistry. Evaluation was carried out on a fluorescence microscope, the VGLUT2-positive cells being counted out both manually and by digital image analysis (MCID).

FIG. 15 show a comparison of the content of VGLUT2-positive cells after rVGLUT2-plasmid transfection without and with siRNA-co-transfection, and a comparison of the two methods of evaluation. The results of the time-saving digital counting (B) agree with the manual count (A) with their relative conditions. All subsequent experiments were therefore evaluated digitally. After treatment with siRNAs directed specifically against VGLUT2, the content of VGLUT2-positive cells was significantly reduced in comparison with cells not treated with siRNA (24.92±1.9) (si-rVGLUT2 100-120 EGT 11.4±2.2 p<0.01; si-rVGLUT2 100-120 AMB 9.84±1.1 p<0.01; si-rVGLUT2 166-186 AMB 6.29±1.1 p<0.001).

On the other hand, the siRNAs against VGLUT1 and against VGLUT3 and the mismatch siRNA do not significantly (p>0.05) influence the content of the VGLUT2-positive cells (A).

If the efficiency of the siRNAs used is calculated from these results as a percentage of the reduction of VGLUT2-positive cells, based on the VGLUT2 expression without the influence of siRNA treatment, the result shown in FIG. 16 is obtained.

It may be seen that the siRNAs directed against VGLUT2 have high efficiency in comparison with ineffective mismatch siRNA. They reduce the content of VGLUT2-expressing cells by 79 to 82%, the self-produced siRNAs acting more effectively in the concentrations used than the siRNA synthesized at Eurogentec. Whereas the siRNA against VGLUT3 and the mismatch siRNA do not exert a significant effect on VGLUT2 protein expression, the siRNA directed against VGLUT1 with an efficiency of ˜47% appears to act non-specifically on VGLUT2 expression. However, the siRNAs against VGLUT2 are significantly more efficient (p<0.001) than the siRNA against VGLUT1.

The transfection experiment has been modified hereinafter: the wt-PC12 cells were treated with the various siRNAs 6 hours before transfection with rVGLUT2 plasmid so the siRNAs were already in the cells at the moment of DNA transfection.

FIG. 17 shows the proportion of VGLUT2-positive cells in this test batch. As with co-transfection, the siRNAs against VGLUT2 lead to a significant reduction (p<0.01) in VGLUT2-expressing cells in comparison with the cultures that were treated with mismatch siRNA. On the other hand, the proportion of VGLUT2-positive cells is not significantly altered (p>0.05) by treatment with siRNAs against VGLUT1 and VGLUT3.

The influence of the existing VGLUT2 protein level on the suppression efficiency of the siRNAs was tested in a further experiment. This model corresponds rather to the endogenously VGLUT2-expresing cells and the situation in vivo. For this purpose the wt-PC12 cells were transfected with the rVGLUT2 plasmid 24 hours before the siRNA treatment. The proportion of VGLUT2-positive cells was determined after a further 24 hours.

FIG. 18 shows the proportion of VGLUT2-positive cells after immunocytochemical labelling. As in the previous experiments, the control siRNAs (against VGLUT1 and VGLUT3, mismatch siRNA) do not lead to a significant reduction (p>0.05) in VGLUT2 protein expression. On the other hand, the siRNAs directed specifically against VGLUT2 (from Ambion) significantly reduce the proportion of VGLUT2-positive cells.

FIG. 19 shows the efficiency of the siRNAs in this batch of experiments. This diagram also shows that the siRNAs directed against VGLUT2 significantly reduce the proportion of VGLUT2-positive cells and that this effect is highly significant in comparison with the mismatch siRNA (p<0.001).

The results of these experiments demonstrate the specific effectiveness of the siRNAs directed against VGLUT2 (si-rVGLUT2 100-120 EGT; si-rVGLUT2 100-120 AMB; si-rVGLUT2 166-186 AMB). There is no difference between the self-produced siRNA (si-rVGLUT2 100-120 AMB) and the siRNA synthesized by Eurogentec (si-rVGLUT2 100-120 EGT). The siRNA controls against VGLUT1 (si-rVGLUT1 739-759 EGT) and against VGLUT3 (si-rVGLUT3 220-240 EGT) do not lead to a reduction in the VGLUT2 protein level. What is known as the mismatch siRNA (si-rVGLUT2 MM EGT), which has the same nucleotides as the specific siRNA against VGLUT2, but in a random and therefore non-complementary arrangement, does not influence the VGLUT2 protein level either.

In these experiments, the efficiencies of the specific siRNAs lie between ˜15 and ˜80%. High siRNA efficiencies (78 to 82%) are achieved with simultaneous co-transfection of siRNA and DNA, whereas lower efficiencies (15 to 23%) are achieved in the suppression experiments with existing VGLUT2 protein levels at the moment of siRNA transfection. This shows that, firstly, the specific siRNAs highly efficiently reduce VGLUT2 protein formation and, secondly, the VGLUT2 proteins are very stable and have only a low turnover.

### Example 3

### Characterization of VGLUT Expression in Primary Cultures of the Spinal Ganglion

The spinal ganglia from neonatal rats were prepared and cultivated as described under the experimental conditions recited in more detail after the practical examples. 20 to 30 respective spinal ganglia from 8 to 16 neonatal rats were prepared for the cultures and the cells were purified. The proportions of neurons in the total number of cells was significantly increased relative to the standard procedure by purification over a BSA column, and by plating out the cell suspension onto poly-L-lysine-coated materials (Grothe and Unsicker, 1987). The neurons, which are much larger and therefore heavier than the non-neuronal cells, settle more rapidly on the coated support. The number of non-neuronal cells, which present predominantly as spindle-shaped cells with branches, is reduced after only 5 min by removing the supernatant. After 4 to 10 days in vitro, the cells were fixed and characterized immunocytochemically.

FIG. 20 shows the cultivated cells of the spinal ganglion. The various cell types may be distinguished by their morphology using a light microscope (A). The neurons may be detected by their spherical configuration and the clear optical refraction while the majority of non-neuronal cells are spindle-shaped fibroblasts. The neurons could also be depicted immunocytochemically with primary antibodies against the pan-neuronal label, “protein gene product 9.5” (PGP 9.5) (B). The cultures also contained a few Schwann cells which were identified by antibodies against GFAP (C).

As the primary sensory nociceptive neurons would be of particular interest for the subsequent experiments, immunocytochemical labels indicated that this neuron population is present in the primary culture and may be cultivated in vitro for at least 8 days. FIG. 21 shows these neurons: peptidergic spinal ganglion cells (A) with CGRP protein expression and heat-sensitive neurons which express the ion channels (B) TRPV1 and (C) TRPV2.

FIG. 22 shows the expression of VGLUT2 (A-C, green signal) in cells of the DRG primary culture. VGLUT2 is expressed in neurons therein (B), all VGLUT2 positive cells also being positive for the neuron label PGP9.5 (yellow signal) but not all PGP9.5-positive cells being VGLUT2-positive (red signal). Co-expression of VGLUT2 in Schwann cells could be ruled out by co-labelling with the used antibodies against GFAP (C, red signal).

Protein expression behaves in a corresponding manner for VGLUT1 (not shown): VGLUT1 is expressed in a subpopulation of PGP9.5-positive neurons and does not occur in Schwann cells.

The expression of the vesicular glutamate transporters VGLUT1 and VGLUT2 was then investigated in peptidergic CGRP-positive neurons (FIG. 23): VGLUT1 (green) and CGRP (red) are expressed in two different cell populations (C, F), whereas VGLUT2 and CGRP coexist (I, M). It can be seen that all CGRP immune-reactive neurons have the vesicular glutamate transporter VGLUT2. However, not all VGLUT2-positive neurons form the neuropeptide CGRP.

Double immunofluorescence for VGLUT2 and TRVP1 was carried out in order to investigate VGLUT2 expression in polymodal nociceptors. As shown in FIG. 24, all TRPV1-positive neurons use the vesicular glutamate transporter VGLUT2 (C, D). VGLUT2 was found predominantly in the cell soma and the axon (D). FIG. 24 (E, F) shows the frequency distribution of the cell sizes of the VGLUT2-positive neurons (E) and the TRPV1-positive neurons (F). It can be seen that TRPV1-postive neurons, with an average cell size ˜180 μm², make up a sub-population of the smaller and medium-sized VGLUT2-positive neurons which have an average cell size of ˜200 μm².

### Example 4

### In vivo Experiments on the Effectiveness of siRNA Against VGLUT2 During Pain Treatment in vivo

Bennett's pain model of the rat was used for this purpose. The analgesic effect of the siRNA according to the invention was investigated in vivo in the rat model. An SiRNA directed against the target sequence of VGLUT2 AAGCAGGATAACCGAGAGACC was used as the active component for this purpose. The two strands of this double-stranded siRNA have the following sequences: r(GCAGGAUAACCGAGAGACC)dTT and r(GGUCUCUCGGUUAUCCUGC)d (TT).

The control siRNA is directed against the following target sequence (AACGACTAGCAAAGCGAGCCA) (no VGLUT2 sequence). The strands of the double-stranded control siRNA each have the following sequences: r(GGACUAGCAAAGCGAGCCA)d(TT) and r(UGGCUCGCUUUGCUAGUCC)d(TT).

All the aforementioned sequences were chemically synthesized (Xeragon, Germantown, SA) and their purity checked by MALDI-TOF analysis. The sequences were dissolved in 0.9% NaCl solution.

Neuropathic pain occurs inter alia after damage to peripheral or central nerves and may accordingly be induced and observed by intentional lesions to individual nerves in animal experiments. Bennett's nerve lesion (Bennett and Xie 1988) Pain 33: 87-107) is one animal model. In Bennett's model, the sciatic nerve is provided unilaterally with loose ligatures. The development of signs of neuropathic pain is observed and may be quantified by thermal or mechanical allodynia.

For this purpose, five male Sprague-Dawley rats (Janvier, France) weighing 140 to 160 g were initially anaesthetized with Pentobarbital (50 mg per kg body weight of the rat Nembutal®, i.p. Sanofi, Wirtschaftsgenossenschaft deutscher Tierärzte eG, Hanover, Germany). One-sided multiple ligatures were then formed on the right-hand main sciatic nerve of the rat. For this purpose the sciatic nerve was exposed halfway along the femur and four loose ligatures (softcat® chrom USP 4/0, metric2, Braun Melsungen, Germany) were bound round the sciatic nerve in such a way that the epineural circulation of blood was not interrupted. The date of the operation was day 1. Measurements were taken I week after ligature of the nerve.

The allodynia was tested on a metal plate of which the temperature was controlled to 4° C. by means of a water bath. To check the allodynia, the rats were placed on the cold metal plate, which was located in a plastic cage. The frequency with which the animals flinched violently from the cooled metal plate with their damaged paw was then counted over a period of 2 minutes prior to application of a solution (preliminary value). The solutions containing 3.16 μg (5 μl) of siRNA according to the invention in 15 μl NaCl or 3.16 μg (5 μl) control RNA (sense strand of the siRNA) in 15 μl NaCl i.t. or NaCl solutions (5 μl) were then applied for control purposes after a single acute intrathecal application under ether narcosis, and the number of retraction reactions was again counted for 2 min after 60 min in each case (test value). The measurements were taken on 4 successive days at respective intervals of 24 hours (days 2-5) for 3 different respective doses (0.001, 0.01 and 0.1 μg/animal). Animals to which pure NaCl solution was applied were used in the experiments both with siRNA and with control RNA as a comparison group.

The siRNA according to the invention against VGLUT2 showed a pronounced analgesic effect in this pain model, namely clear inhibition of cold allodynia without dose dependency with the best effect with the lowest dose group (1 ng/animal). With the highest dose, the animals showed increased spontaneous activity both in the control group and in the verum group. This could be the reason for the weaker effect in the high dose group. There were no further side effects (cf. FIG. 28).

The materials and methods used and mentioned in the aforementioned practical examples and specific experimental conditions are described in more detail hereinafter:

1. Materials Used

1.1 Experimental Animals

All adult experimental animals were male or female Wistar rats (300 g) and were obtained from Charles River (Sulzfeld) and from the German Experimental Animal Institute (Hanover). The animals were kept in a 12 h/12 h day/night rhythm with free access to food and water. At least 4 days, in which the health of the animals was monitored, elapsed between supply of the animals and the beginning of the experiment. The neonatal rats originated from an individual breed of male and female Wistar rats which were covered at regular intervals. All neonatal animals were used for the production of primary cultures from P0 (postnatal day 0) to P5.

1.2 Cell Lines

wt-PC12

The immortal tumour cell line PC12 was isolated from a tumour of the adrenal marrow of the rat in 1976 (Greene and Tischler, 1976). The cells grow in a non-adherent manner, lead to transplantable tumours in rats and react reversibly to NGF (nerve growth factor) with the formation of neuron-like projections. The PC12 cell line was made available to Grünenthal's laboratory (Aachen), which, in turn, obtained the cell line from ATCC. The cells were cultivated in a modified DMEM medium. These PC12 cells are designated hereinafter as wtPC12 (wild type) for better distinction.

PC12 MR-A and PC12 MR-B

These two cell lines are mutated or differentiated variants of the wt-PC12 cells which were originally obtained from Dr. Reiner Westermann (Institute for Anatomy and Cell Biology, Marburg) from ATCC. In contrast to their wt mutants, the cells grew adherently, and were flat and spindle-shaped and formed projections without the addition of NGF. References in the literature lead to the assumption that these variants could form a glutamatergic phenotype (Jimenez et al, 2003; Zheng et al, 1996).

1.3 Bacterial strains

| | |
|---|---|
| E. coli strain DH5α | Clontech (Heidelberg) |
| E. coli strain Xl-1 blue | Clontech (Heidelberg) |

1.4 Apparatus
Centrifuges

| | |
|---|---|
| Biofuge pico type table centrifuge | Heraeus (Hanau) |
| Labofuge III | Heraeus (Hanau) |
| Cooling centrifuge, 5043 | Eppendorf (Hamburg) |
| JS21 centrifuge | Beckmann (Munich) |

Incubators

| | |
|---|---|
| Incubation oven (16/37° C.) | WTB Binder (Reiskirchen) |
| Incubator (37° C./5% CO₂) | Heraeus (Hanau) |
| Incubator (37° C./5% CO₂) | Heraeus (Hanau) |

Electrophoresis

| Agarose gel electrophoresis equipment | Kodak/Integra (New Haven) |
|---|---|
| Gel documentation equipment, Gel Doc | BIO-RAD (Munich) |
| 1000 | |

Microscope and digital image analysis

| | |
|---|---|
| Confocal laser scanning microscope | Olympus (Hamburg) |
| AX70 Microscope | Olympus (Hamburg) |
| IX70 Microscope | Olympus (Hamburg) |
| Olympus SZH10 research stereo | Olympus (Hamburg) |
| MCID M4 image analysis system | Imaging Research (St. Catherine's, |
| | Canada) |
| SPOT camera | Diagnostics Instruments Inc. |
| | (Seoul, Korea) |
| SPOT image analyses (Version 3.4) | Diagnostics Instruments |
| | Inc. (Seoul) |

Other equipment and consumables

| | |
|---|---|
| Suction device | Neo-Lab, Vogel (Heidelberg) |
| Autoclave | Integra BioSciences Inc. (Woburn, USA) |
| Autoradiographic cassette | Amersham (Little Chalfont, England) |
| Bacteria shaker | Certomat H Braun Biotech (Melsungen) |
| Cover slips | Menzel (Braunschweig) |
| Disposable pipettes | Greiner bio-one (Frickhausen) |
| Tissue culture bottles | Cellstar ® Greiner bio-one (Frickhausen) |
| Heating plate | Medax (Kiel) |
| Piston pipettes | Eppendorf (Hamburg) |
| Cryotubes | Nalge Nunc International (Rochester USA) |
| Cryostat CM3050 | Leica (Nussloch) |
| Neubauer counting chamber | Marienfeld (Lauda-Konigshofen) |
| Object carrier | Menzel (Braunschweig) |
| Peltier thermal cycler PTC-200 | M J Research (Watertown, USA) |
| pH Meter 766 | Knick (Berlin) |
| Petridishes | Nalge Nunc International (Rochester, USA) |
| Pipette tips | Eppendorf (Hamburg) |
| Plastic baths | Neolab, (Heidelberg) |
| Water purifier (Milli-Q) | Millipore (Billerica, USA) |
| Speedvac (cold trap) | Heraeus (Hanau) |
| Stereotactic apparatus | David Kopf Instruments (Tujunga, USA) |
| 1040 polymax tumbler | M J Research (New York, USA) |
| Thermometer | IKA Labortechnik (Staufen) |
| Circulating air oven | Memmert (Schabach) |
| Video printer | Mitsubishi Electric (Tokyo, Japan) |
| Vortex: VF2 and MS2 Minishaker | IKA (Staufen) |
| Vortex Genie 2 | Bender & Hohenheim AG (Zurich, Switzerland) |
| 1012 MP balance | Sartorius (Gottingen) |
| Julabo 5 water bath | Julabo (Seelbach) |
| Water bath | Memmert (Schabach) |
| Nunclon ™ cell culture dishes (Jun. 24, 1996 well) | Nalge Nunc International (Rochester, USA) |

1.5 Chemicals

Any chemicals not listed in detail here were obtained from Fluka (Buchs, Switzerland), GibcoBRL (Eggenstein), Merck(Darmstadt), Riedel de Haen (Seelze), Roche (Basel, Switzerland), Roth (Karlsruhe), Serva (Heidelberg) and Sigma (Deisenhofen), unless stated otherwise in the method section.

| | |
|---|---|
| Bacto Agar | Difco (Detroit, USA) |
| Bacto Tryptone | Difco (Detroit, USA) |
| Bacto Yeast Extract | Difco (Detroit, USA) |
| Fixing solution Formamide | BDH (Poole, England) |
| NAOH | Baker (Deventer, Holland) |
| Ketamine | Parke-Davis (Freiburg) |
| Paraffin | Vogel Histo-Comp |
| x-GAL | Invitrogen (Karlsruhe) |
| Xylazin | Bayer (Leverkusen) |

1.6 Buffer and Solutions

| | |
|---|---|
| H₂O MilliQ | purified and autoclaved |
| PBS (phosphate buffered saline): | 6.5 mM Na₂HPO₄ |
| | 1.5 mM KH₂PO₄ |
| | 2.5 mM KCl |
| | 140 mM NaCl pH 7.25 |
| 6 x sample buffer (DNA/RNA): | 50% (w/v) glycerine |
| | 1 nM EDTA |
| | 0.4% (w/v) bromophenol blue |
| | 0.4% (w/v) xylene cyanol |
| TE (tris EDTA) | 10 mM tris-base |
| | 1 mM EDTA pH 8.0 |
| TAE (tris-acetate-EDTA): | 1 x TAE 40 mM tris base, 2 mM EDTA |
| | 50x |
| | 242 g tris base |
| | 57.1 ml glacial acetic acid |
| | 37.2 g Na₂EDTA × H₂O |
| | ad 1 H₂O pH 8.5 |

1.7 Media and Media Additives

I. Basic Media

All media were obtained from GibcoBRL (Eggenstein):

DMEM (Dulbecco's modified Eagle Medium) with a high glucose content

| | |
|---|---|
| Ham's F12 | |
| HBSS (10x) Ca²⁺/Mg²⁺-free | |
| OptiMEM (serum-reduced) | |
| RPMI 1640 | (Roswell Park Memorial Institute) |

II. Media Additives

| | |
|---|---|
| Antibiotic mix | GibcoBRL (Eggenstein) |
| Cytosine arabinoside | Sigma (Deisenhofen) |
| Foetal calf serum | Sigma (Deisenhofen) |
| HyClone FBS | GibcoBRL (Eggenstein) |
| HAT supplement | GibcoBRL (Eggenstein) |
| Glutamine | GibcoBRL (Eggenstein) |
| Horse serum | GibcoBRL (Eggenstein) |
| Nerve growth factor NGF 7S | Sigma (Deisenhofen) |

Composition of the Growth Media

Medium for PC12 cells: RPMI with

10% Horse serum

5% FCS

1% Glutamine (200 mM)

1% Antibiotic mix (100×)

Medium for spinal ganglion cells: DMEM with

10% FCS

1% Antibiotic mix (100×)

1% Glutamine

10 μM Cytosine arabinoside

25 ng/ml NGF

1.8 Enzymes and other proteins

| | |
|---|---|
| Collagenase (267 U/ml) | Seromed, Biochrom (Berlin) |
| Dispase | GibcoBRL (Eggenstein) |
| DNA-Polymerase AmpliTaq Gold ™ | Applied Biosystems (Foster |
| | City, USA) |
| DNase (LS002139) | Worthington |
| Poly-L-Lysine, Hydrobromide | Sigma (Deisenhofen) |
| Poly-D-Lysine, MG >300 kDa | Sigma (Deisenhofen) |
| Restriction endonucleases | Boehringer (Mannheim) |
| RNA-polymerases (SP6, T3, T7) | Boehringer (Mannheim) |
| RNase-free DNase 1 | Boehringer (Mannheim) |
| RNase (A, T1) | Boehringer (Mannheim) |
| RNAsin | Fermentas (Vilnius, |
| | Lithuania) |
| Superscript ™ 11 H-Reverse Transcriptase | GibcoBRL (Eggenstein) |
| T4 DNA-Ligase | Biolabs (Beverly, USA) |
| Trypsin/EDTA (0.5%/0.2%) | GibcoBRL (Eggenstein) |
| Trypsin | GibcoBRL (Eggenstein) |

1.9 Nucleic Acids and Vectors

| | |
|---|---|
| 100 bp-DNA-ladder | GibcoBRL (Eggenstein) |
| 1 kb-DNA-ladder | GibcoBRL (Eggenstein) |
| ATP, CTP, GTP, UTP | GibcoBRL (Eggenstein) |
| Transcript vectors (pGEM-T) | Promega (Madison, USA) |
| Expression vectors (pCR3.1) | Invitrogen (Karlsruhe) |
| Oligo (dT) 15-18 primer | Boehringer (Mannheim) |

1.10 Oligonucleotides

All oligonucleotides were designed with the oligo 4.0 programme itself, checked for undesirable sequence homology using BLAST and the synthesis ordered from MWG-Biotech(Ebersberg).

| PCR primer | | |
|---|---|---|
| rVGLUT1 | | |
| rVGluT1(2_4)F | 5′-TCTGGGTTTCTGCATCAGC-3′ | PCR product size: 153 bp |
| rVGluT1(2_4)R | 5′-CCATGTATGAGGCCGACAGT-3′ | |
| rVGLUT2 | | |
| rVGluT2(8_9)F | 5′-AAGACCCCATGGAGGAAGTT-3′ | PCR product size: 184 bp |
| rVGluT2(8_9)R | 5′-ATTGTCATGACCAGGTGTGG-3′ | |
| rVGLUT3 | | |
| rVGluT3(4_5)F | 5′-ATCCAGAGACGGTGGGTCTT-3′ | PCR product size: 185 bp |
| rVGluT3(4_5)R | 5′-ATGACACAGCCGTAATGCAC-3′ | |
| rGAPDH | | |
| rGAPDH(7_8)F | 5′-ATCCTGGGCTACACTGAGGA-3′ | PCR-Product size: 162 bp |
| rGAPDH(7_8)R | 5′-ATGTAGGCCATGAGGTCCAC-3′ | |

| Primer for siRNA-synthesis using siRNA-construction | |
|---|---|
| kit (Ambion): | |
| For siRNA si-rVGLUT2 100-120 AMB | |
| rVGluT2 TS10 as | 5′-AAGCAGGATAACCGAGAGACCCCTGTCTC-3′ |
| rVGluT2 TS10 s | 5′-AAGGTCTCTCGGTTATCCTGCCCTGTCTC-3′ |
| For siRNA si-rVGLUT2 166-186 AMB | |
| rVGluT2 TS14 as | 5′-AAGGCTCCGCTATGCGACTGTCCTGTCTC-3′ |
| rVGluT2 TS14 s | 5′-AAACAGTCGCATAGCGGAGCCCCTGTCTC-3′ |

1.11 Kits

| | |
|---|---|
| qPCR Core kit for SYBR ® Green | Eurogentec (Liège, |
| | Belgium) |
| RNeasy Mini Kit | QIAGEN (Hilden) |
| Silencer ™ siRNA Construction Kit | Ambion (Austin, USA) |
| Silencer ™ siRNA Labelling Kit | Ambion (Austin, USA) |
| QIAamp DNA Mini Kit | QIAGEN (Hilden) |
| QIAprep spin MAxiprep Kit | QIAGEN (Hilden) |
| QIAprep spin Miniprep Kit | QIAGEN (Hilden) |
| QIAquick Gel Extraction Kit | QIAGEN (Hilden) |
| QIAquick Nucleotide Removal Kit | QIAGEN (Hilden) |
| QIAquick PCR Purification Kit | QIAGEN (Hilden) |
| Vectastain Elite Avdin-Biotin-Blocking Kit | Vector Laboratories |
| Vectastain Elite ABC Kit | Vector Laboratories |

1.12 Antibodies and Detection Systems

The various cell types were detected with primary antibodies against generally recognized, readily characterized epitopes (labels) in the respective cell type. These are PGP9.5 for neurons, GFAP for astrocytes and S100 for oligodendrocytes. In addition to these, further antibodies against specific proteins were used. The optimum concentrations of the primary antibodies were titrated out in each case. Table 1 gives an overview of the primary antibodies used and also the working dilutions thereof.

The primary antibodies were then detected by fluorochrome-coupled secondary antibodies or fluorochrome-coupled streptavidin (Table 2) which interacted with species-specific biotinylated antibodies. Cy3 (red fluorescence) or Alexa 488 (green fluorescence) were used as fluorochromes.

TABLE 1
List of primary antibodies used

| Antigen | Internal | Source | Dilution | Species |
|---|---|---|---|---|
| β-III Tubulin | Tubulin | DPC/Biermann | 1:4 | Mouse, |
| | | Bad Nauheim | | monoclonal |
| CGRP | CGRP/Ste | Fred Nyberg, | 1:8000 | Rabbit |
| | | Stefan Persson | | |
| GFAP | GFAP-Dako | DakoCytomation | 1:5000 | Rabbit |
| | | Denmark | | |
| GFAP | GFAP | Boehringer | 1:4 | Mouse, |
| | | (Mannheim) | | monoclonal |
| PGP9.5 | PGP | UItraClone | 1:1500 | Rabbit, |
| | | (Wellow, England) | | polyclonal |
| S100 | S100 | Biogenesis | prediluted | Rabbit, |
| | | (Poole, England) | | polycllonal |
| Substance P | SP | Lee Eiden (NIH, | 1:1000 | Rabbit |
| | | USA) | | |
| TRPV1 | Caps. receptor (SA 6583) | Eurogentec | 1:250 | Rabbit |
| | | (Belgium) | | |
| TRPV | VRL-1 | Chemicon | 1:400 | Rabbit, |
| | | Temecula, USA | | polyclonal |
| VGLUT1 | BNPI/BC66 | J. Erickson (New | 1:600 | Rabbit |
| | | Orleans) | | |
| | | (Varoqui et al., 2002b) | | |
| VGLUT1 | BNPI/p437/7/01 | J. Erickson (New | 1:800 | Guinea pig |
| | | Orleans) | | |
| | | (Varoqui et al., 2002b) | | |
| VGLUT2 | NPI/p438/7/01 | J. Erickson (New | 1:800 | Guinea pig |
| | | Orleans) | | |
| | | (Varoqui et al., 2002b) | | |
| VGLUT2 | DNPI/DC68 | J. Erickson (New | 1:100 | Rabbit |
| | | Orleans) | | |
| | | (Varoqui et al., 2002b) | | |
| VGLUT3 | VGLUT3/447/b19 | J. Erickson (New | 1:600 | Guinea pig |
| | | Orleans) | | |
| | | (Schafer et al., 2002) | | |
| VGLUT3 | VGLUT3/94/b15 affi. | J. Erickson (New | 1:50 | Rabbit |
| | | Orleans) | | |
| | | (Schafer et al., 2002) | | |
| VGL | VGLUT3 | Chemicon | 1:5000 | Guinea pig, |
| UT3 | Chemicon | (Temecula, USA) | | polyclonal |

The biotinylated Isolectin B4 (Sigma, Deisenhofen) was also used in a concentration of 1:20.

TABLE 2
List of secondary antibodies used

| Antibody | Source | Dilution | A. D |
|---|---|---|---|
| Anti-guinea pig-IgG-Cy3 | Dianova (Hamburg) | 1:100 | Donkey |
| Anti-mouse-IgG-Cy3 | Dianova (Hamburg) | 1:100 | Donkey |
| Anti-rabbit-IgG-Cy3 | Dianova (Hamburg) | 1:100 | Donkey |
| Anti-guinea pig-IgG-A488 | Dianova (Hamburg) | 1:100 | Donkey |
| Anti-mouse-IgG-A488 | Dianova (Hamburg) | 1:100 | Donkey |
| Anti-rabbit-IgG-A488 | Dianova (Hamburg) | 1:100 | Donkey |
| Avdin-biotin-peroxidase | Boehrinher | 1:500 | Donkey |
| Streptavidin-Alexa 488 | MoBiTec (Gottingen) | 1:200 | Donkey |

2. Methods

2.1 Removal and Treatment of Tissue

The animals were killed by CO₂ inhalation and subsequent decapitation. The tissue required for the various purposes was then removed in different ways:

Nucleic Acid Extraction:

After removal, the tissue was spread over ice, placed in a cryotube and, after shock-freezing in liquid nitrogen, the tissue was initially stored on dry ice and then at −70° C.

Primary Culture:

For the application of primary cultures, the tissue (spinal ganglia) was immediately removed on self-produced ice-cooled preparation dishes and initially collected in 1× CMF medium for further treatment. The calcium-free and magnesium-free 1× CMF medium consisted of 10% HBSS (10×), 1% antibiotic mix and 0.2% phenol red (0.5%). The pH was titrated with bicarbonate (7.5%) and could be detected by the cherry-red color of the indicator.

2.2 Application of Primary Cultures

For application of the primary cultures, neonatal rats from stages P0 to P5 were used for the spinal ganglion cell culture and P0 to P3 for the cerebellum cultures. The animals were disinfected with alcohol and killed by decapitation with sterile shears.

2.2.1 Neuronal Primary Cultures of Spinal Ganglia

The spinal ganglia were prepared by C-Grothe's modified procedure (Grothe and Unsicker, 1987).

Dissection of the Spinal Ganglia

The body of the killed rat was fixed ventrally on a cork board and the vertebral column exposed by removing the skin and the muscles at the back of the neck and shoulders. The vertebral column was then opened from the caudal end to the cranial end and the bone marrow displayed. In the juvenile animals, the bone marrow was left in the spinal canal and the opened vertebral column removed in its entirety for fixing in a cooled preparation dish. Only then was the bone marrow carefully removed in steps and the spinal ganglia taken from the exposed intervertebral holes.

The dissected spinal ganglia were collected in a cooled Petri dish with 1× CMF medium until fine preparation. The spinal ganglion was cleaned of nerves, connective tissue and blood residues under the binocular device, and was then transferred into an ice-cooled tube filled with 1× CMF medium.

Dissociation and Purification of the Spinal Ganglion Cells

After removal of the medium, the spinal ganglia were incubated for 30 to 45 min at 37° C. and 5% CO₂ for chemical dissociation with an enzyme mixture of 0.075% collagenase and 0.15% dispase in CMF medium. Adhesion of the ganglia was to be prevented by repeated shaking during incubation. Half of the enzyme mixture was removed on completion of incubation and the same volume of a 0.25% trypsin solution fed to the remaining residue. After incubation for 15 to 25 min at 37° C. and 5% CO₂, the enzyme solution was removed to 300 μl and the ganglia in this volume were mechanically dissociated. Three siliconized sterile Pasteur pipette orifices of different sizes, produced under the Bunsen burner flame were used for this purpose. By increasingly reducing the orifice diameter, a uniform cell suspension of the spinal ganglia could be produced. The cell suspension was finally centrifuged for 5 min at 1000 rpm, the supernatant discarded and the cell pellet resuspended in ˜1 ml CMF. To concentrate the culture with neuronal cells, the cell suspension was coated over a 20% BSA column (20% BSA w/v in 0.5 ml CMF medium) and then centrifuged for 5 min at 1000 rpm. The medium was suction filtered to ˜100 μl and resuspended in 1 ml fresh medium. The later culture medium (DMEM/FCS) was used for this purpose.

Plating Out of the Cells

After determining the number of cells, the spinal ganglion cells were distributed over the culture vessels, according to the subsequent use, and cultivated at 37° C. in 5% CO₂. A glucose-rich DMEM medium with various additives was used for cultivation purposes (exact composition described under the heading: Cell Culture). In addition to glutamine and an antibiotic mix, the nerve growth factor NGF 7S was supplied in a concentration of 25 ng/ml, as the survival and the differentiation of the neonatal neurons of the spinal ganglion are NGF-dependent. In addition, the mitose inhibitor, cytosine arabinoside, was added in a concentration of 10 μM to the medium to prevent proliferation of the non-neuronal cells. The plated out spinal ganglion cells could be cultivated without difficulty for up to 2 weeks by changing the medium every 2 to 3 days.

2.3 Eukaryotic Cell Culture

Cultivation and Passaging of Eukaryotic Cells

All the aforementioned cell lines work best in their respective growth medium at 37° C. in the gasification incubator at 95% relative atmospheric humidity and 5% CO₂. Media and solutions were heated to 37° C. prior to use.

The adherently growing cell lines (F-11, wt-PC12 MR-A, PC12 MR-B) were routinely split every 4 to 6 days. For this purpose, they were treated with trypsin/EDTA solution (0.05% trypsin, 0.02% EDTA in PBS) for a few minutes at 37° C. (microscopic control). Trypsin is a proteolytic enzyme which hydrolyses peptide bonds of cell/cell bonds in which the carbonyl group is taken from the lysine or arginine. Slight traces of medium can impair the effect of trypsin, which is why the cells had to be washed with phosphate-buffered calcium-free and magnesium-free salt solution (PBS) prior to the trypsin treatment. The trypsin used could be inactivated again by addition of medium with addition of FCS. The chelator EDTA binds calcium which is required by some cell/cell bonds.

Once the cells had dissolved, they were transferred with serum-containing medium from the culture vessel into a Falcon tube. After centrifuging and suction filtering the old medium, the cell pellets were resuspended in 5 ml fresh medium and plated out into the respective culture vessels in their growth medium, depending on the subsequent use.

The wt-PC12 cells are suspension cells. For passaging, the culture vessels were placed obliquely so the wt-PC12 cells growing in grape-like heaps sedimented gradually in a corner of the vessel. The supernatant, which also contained the lighter cell debris, was removed and the cells transferred into a Falcon tube with fresh medium. The suspension was then centrifuged for 5 min at 1000 rpm, the supernatant was removed and the cells were plated out in fresh growth medium.

Freezing of Eukaryotic Cells

The cells were frozen in DMSO-containing medium in liquid nitrogen for long-term storage, in order to protect them from genetic modification and to minimize the risk of contamination. Without the addition of reagents, which act as cryoprotection for the cells, most mammalian cells die when frozen. The mortality of cells is minimized by DMSO in the medium, as the freezing point is lowered and the cooling process therefore decelerated.

After separation with trypsin/EDTA solution, the cells were initially washed with PBS and the number of cells determined using the Neubauer counting chamber. The amount of cells to be frozen was placed in a Falcon tube, and the supernatant discarded after centrifugation at 1000 rpm. The cell pellet was finally resuspended in the freezing medium (90% growth medium, 10% DMSO) and aliquoted in Nunc tubes with 2 ml in each case (for example with 2 million cells). The tubes were frozen at −80° C. for several hours in a cryofreezing unit with a cooling rate of 1° C. per minute and were then stored in liquid nitrogen.

Revitalization of Eukaryotic Cells

In some cases, cells were recultivated. For this purpose, they were heated rapidly to 37° C. in a water bath after removal from the nitrogen tank (−196° C.). The cell suspension was removed, transferred into 5 ml preheated medium and sedimented in the centrifuge (3 min, 200×g). The medium was suction filtered, the target pellet resuspended in fresh medium and transferred into a cell culture dish. On the next day, the cells were washed with PPS and supplied with fresh medium.

Determination of Number of Cells

A Neubauer counting chamber was used to determine the number of cells. For this purpose, a drop of the cell suspension was applied to the Neubauer counting chamber and the number of cells (Z=cells/0.01 mm²) counted out under the microscope from four corner squares and the number of cells per ml determined (number of cells=Z×2500) (Amiri et al.).

Mycoplasm Test

All cell lines were tested at regular intervals for mycoplasm contamination. Mycoplasms are obligate parasitic bacteria. They are wall-less, very small intracellular parasites and cannot propagate independently of the host cell. As they have only one cell membrane, but no bacterial wall of murein, they do not have a fixed form and are insensitive to penicillin. Their size varies between 0.22 and 2 μm. Filtration through a membrane with a pore size of 0.1 μm allows separation of mycoplasm. Contamination with mycoplasm may be detected most rapidly by staining the mycoplasm DNA with the fluorochrome DAPI (4-6-diamidino-2-phenylindol-di-hydrochloride) which binds specifically to DNA. In the case of mycoplasm contamination of cell cultures, individual fluorescing points are found in the cytoplasm and sometimes also in the intercellular space.

For the mycoplasm test, a corresponding amount of DAPI stock solution (1 mg/ml, 10 mg DAPI dissolved in 10 ml water, aliquoted and stored at −20° C.) was diluted with methanol to a working concentration of 1 μg/ml (stable for about 6 months at 4° C.). To stain the cells, the cells cultivated on cover slips or Petri dishes were washed once with the working solution and incubated for 15 min at ambient temperature with the working solution. The solution was washed once with methanol and the cover slips were embedded in a drop of glycerine or PBS then evaluated under the fluorescence microscope.

Coating of Culture Vessels

For certain experiments, the culture vessels or cover slips were coated to assist adhesion of the cells. As coating materials, poly-L-lysine (0.1 mg/ml) was used for wt-PC12 cells or poly-D-lysine (0.5 mg/ml) for the cerebellum culture. The culture vessels were incubated for at least 2 hours at ambient temperature with poly-L-lysine and poly-D-lysine, then washed twice with H₂O and coated with H₂O and stored at 4° C. until use. Whereas the poly-L-lysine coated materials were used in the moist state, the poly-D-lysine coated materials were not used until they had been dried under the sterile bench.

Transfection in Eukaryotic Cells

Transfection is understood to be the introduction of extraneous DNA or RNA into eukaryotic cells by physical or chemical methods. The most important chemical method for the transfer of nucleic acids is lipofection: reagents from cationic lipids form small (100-400 mm) unilamellar liposomes under optimum conditions in aqueous solution. The surface of these liposomes is positively charged and is electrostatically attracted both by the phosphate backbone of the nucleic acids and by the negatively charged cell membrane (Gareis et al., 1991; Gershon et al., 1993; Smith et al., 1993). The nucleic acids are not enclosed within the liposomes, but bind spontaneously to the positively charged liposomes and form DNA/RNA lipid complexes (Felgner et al., 1987). There are indications that the complexes are incorporated via the endosomal or lysosomal pathway (Coonrod et al., 1997).

Transfection of DNA by Liptofectamine™ 2000

For the transfection of DNA in eukaryotic cells, the cationic lipid reagent Liptofectamine™ 2000 (LF2000™) was predominantly used, while adhering to the following general recommendations from the manufacturer, Invitrogen:

A DNA to LF2000™ ratio of 1:2 to 1:3 was recommended for producing DNA-LF2000™ complexes. A cell density of 90 to 95% should exist at the moment of transfection, to achieve high efficiency and a high expression level. Antibiotics were not added during transfection, as they would trigger cell death. Transfection under various conditions was carried out with an EGFP vector to optimize the efficiency of transfection. To measure the efficiency of transfection, the protein expression of the green fluorescing protein GFP was determined semi-quantitatively using a fluorescence microscope.

Tranfection of DNA in wt-PC12 Cells

2.5×10⁵ wt-PC12 cells were plated out in a 24-well plate on poly-L-lysine-coated cover slips in 0.5 ml medium on the day before transfection. The medium was the normal growth medium for wt-PC12 cells, but without the addition of antibiotics. 0.8-1 μg DNA was dissolved in 50 μl OPTI-MEM® for each well to be transfected. In addition, 1-2 μl LF2000™ were diluted in 50 μl OPTI-MEM® for each well and incubated for 5 min at ambient temperature. The dissolved DNA was now mixed with the diluted LF2000™ solution and incubated for 20 min at ambient temperature to form the DNA-LF2000™ complexes, and 100 μl of the complexes then placed directly in the corresponding wells, which were mixed by careful swinging. The cells were cultivated for a further 24-72 hours in the same medium under normal conditions until analysis of the expression.

Transfection of DNA in Spinal Ganglion Cells

For transfection, 5×10⁵ die cells of the freshly purified DRG suspension were used, which were plated out in a 24-well plate on poly-L-lysine-coated cover slips in 0.5 ml growth medium without the addition of antibiotics. 0.8-1 μg DNA was dissolved in 50 μl OPTI-MEM® for each well to be transfected. In addition, 1-2 μl LF2000™ were diluted in 50 μl OPTI-MEM® for each well and incubated for 5 min at ambient temperature. The dissolved DNA was now mixed with the diluted LF2000™ solution and incubated for 20 min at ambient temperature to form the DNA-LF2000™ complexes, and 100 μl of the complexes then placed directly in the corresponding wells, which were mixed by careful swinging. The cells were cultivated for a further 24-72 hours in the same medium under normal conditions until analysis of the expression.

Transfection of siRNA

The transfection of siRNA in eukaryotic cells was carried out using Lipofectamine™ 2000. LF2000™ has already been successfully used for RNAi experiments in mammalian cells by other groups (Gitlin et al., 2002; Yu et al., 2002). As with the transfection of DNA, the optimum transfection conditions had to be determined experimentally. Cy3-labelled siRNA was used for this purpose.

Transfection of siRNA in wt-PC12 Cells

2.5×10⁵ wt-PC12 cells were plated out in a 24-well plate on poly-L-lysine-coated cover slips in 0.5 ml medium on the day before transfection. The medium was the normal growth medium for wt-PC12 cells, but without the addition of antibiotics. 20-100 pmol siRNA were dissolved in 50 μl OPTI-MEM® for each well to be transfected. In addition, 1-2 μl LF2000™ were diluted in 50 μl OPTI-MEM® for each well and incubated for 5 min at ambient temperature. The dissolved siRNA was now mixed with the diluted LF2000™ solution and incubated for 20 min at ambient temperature to form the DNA-LF2000™ complexes, and 100 μl of the complexes then placed directly in the corresponding wells, which were mixed by careful swinging. The cells were cultivated for a further 24-72 hours in the same medium under normal conditions until analysis of the expression.

Transfection of siRNA in Cells of the PC12 MR-A and PC12 MR-B Lines

2.5×10⁵ PC12 cells were plated out in a 24-well plate on cover slips in 0.5 ml medium on the day before transfection. The medium was the normal growth medium for PC12 MR-A/B cells, but without the addition of antibiotics. 20-200 pmol siRNA were dissolved in 50 μl OPTI-MEM® for each well to be transfected. In addition, 1-2 μl LF2000™ were diluted in 50 μl OPTI-MEM® for each well and incubated for 5 min at ambient temperature. The dissolved siRNA was now mixed with the diluted LF2000™ solution and incubated for 20 min at ambient temperature to form the DNA-LF2000™ complexes, and 100 μl of the complexes then placed directly in the corresponding wells, which were mixed by careful swinging. The cells were cultivated for a further 24-72 hours in the same medium under normal conditions until analysis of the expression.

Transfection of siRNA in Cells of the DRG Primary Cultures

For transfection, 5×10⁵ die cells of the freshly purified DRG suspension were used, which were plated out in a 24-well plate on poly-L-lysine-coated cover slips in 0.5 ml growth medium without the addition of antibiotics. 20-200 pmol siRNA were dissolved in 50 μl OPTI-MEM® for each well to be transfected. In addition, 1-2 μl LF2000™ were diluted in 50 μl OPTI-MEM® for each well and incubated for 5 min at ambient temperature. The dissolved siRNA was now mixed with the diluted LF2000™ solution and incubated for 20 min at ambient temperature to form the DNA-LF2000™ complexes, and 100 μl of the complexes then placed directly in the corresponding wells, which were mixed by careful swinging. The cells were cultivated for a further 24-72 hours in the same medium under normal conditions until analysis of the expression.

Toxicity Study

An assay with trypan blue was carried out to investigate the cytotoxic influence of the transfection reagent LF2000™ on the cells. For this purpose, 3.0×10⁴ cells were plated out in a 24-well plate 24 hours before transfection. Transfection was carried out with siRNA negative controls and different amounts of transfection reagents. All cells were washed with PBS 48 hours after transfection and stained with 10% trypan blue. The survival rate was calculated as follows:
Vitality rate=(total number of cells−number of stained cells)/total number of cells×100.

Fixing of Cells

The cells were fixed either for 20 min at ambient temperature in PBS with 3-4% (v/v) paraformaldehyde or for 15 min at ambient temperature with methanol (−20% ° C).

2.4 Microbiology

Competent bacteria were used to amplify double-stranded DNA fragments. For this purpose, the DNA to be amplified was incorporated into plasmids with selection labels and transformed into bacteria. Bacteria were plated out on selective agar plates and corresponding clones were removed and cultured in antibiotic-containing LB medium. The plasmids with insert were later isolated from the propagated bacteria and further processed.

Production of Transformation-Competent Cells

Treatment with ice-cold magnesium and calcium chloride solution enables the bacteria to absorb extraneous DNA spontaneously (transformation). The E. coli strains DH5α and XL-1 blue were used to produce the competent bacterial cells. For this purpose, a cell culture was placed in TYM medium (LB-medium+10 mM MgSO₄) and incubated until an adequate density (OD600 nm=0.4-0.6) was achieved. The cells should be in the log phase. After brief centrifugation and pouring off the supernatant, the pellet was resuspended in 40 ml Tfb1-buffer (30 mM KAc, 50 nM MgCl₂×2H₂O, 100 mM KCl, 10 mM CaCl₂×2H₂O, 15% glycerol) per 100 ml culture. After 45 min incubation on ice, the cells were pelletized again and incorporated in 1/10 volume Tfb2-buffer (10 mM Na-MOPS, 10 mM KCl, 75 mM CaCl₂×2H₂O, 15% glycerol). The competent cells were stored as 100 μl aliquots at −70° C. until use.

Transformation of E. coli

Competent bacteria (approximately 10⁸ clones per μg plasmid DNA) were reacted with plasmid DNA, and incubated for 45 min on ice and just 1 min at 42° C. LB medium (Sambrook et al., 1989) was then added and the mixture incubated for a further 30 min at 37° C. while shaking. The bacteria were separated on selective agar plates (Sambrook et al., 1989) and incubated overnight at 37° C. Precultures (5 ml LB medium with a suitable antibiotic) were inoculated with separated colonies and shaken for several hours at 37° C. Preparatory cultures (100 ml LB with a suitable antibiotic) were inoculated 1:1000 with the precultures and incubated overnight at 37° C. while shaking.

Analytical Plasmid Isolation

In order to isolate the plasmids from the bacteria, the bacteria have to be lysed. The plasmids are then separated by centrifugation of proteins and genomic bacteria-DNA. The plasmids were purified by column chromatography with commercial kits from Qiagen. The bacteria were pelletized by centrifugation for 5 min at 5000×g, the supernatant was discarded and the pellet resuspended in 250 μl buffer P1. The bacteria were lysed by addition of 250 μl buffer P2 and the solution was neutralized by a further 350 μl buffer N3. The mixture was then centrifuged for 10 min (the centrifugation steps were carried out at maximum speed, unless otherwise stated), the supernatant being carefully removed and transferred into a QIAprep column. After 1 min centrifugation, the column was again washed with 750 μl buffer PE and in turn centrifuged for 1 min. The plasmid DNA was eluted into a clean receiver by addition of 30-50 μl H₂O onto the column and subsequent centrifugation for 1 min. For quality control, 5 to 10 ml of this mixture were analysed by restriction digestion and subsequent agarose gel electrophoresis.

Quantitative Plasmid Isolation

Relatively large amounts of plasmid DNA with a high degree of purity for carrying out transfections were isolated using the Qiagen Plasmid Maxi Kit. For this purpose, a preculture was prepared during the day, i.e. a bacterial colony was inoculated in 3 ml ampicillin-containing medium and cultivated for approximately 7 hours in the shaking incubator at 37° C. This preculture was then transferred into 500 ml LB medium and shaken overnight at 37° C. The bacterial suspension was centrifuged off at 4° C. at 3000 rpm the next morning. The resultant bacterial pellet was used for plasmid isolation following the manufacturer's directions.

After absorption of the DNA pellets in sterile H₂O, the concentration was determined platemetrically and the DNA quality checked by restriction digestion and subsequent agarose gel electrophoresis.

2.5 Molecular Biological Methods

2.5.1 Nucleic Acids

Purification of DNA

Nucleic acids may be concentrated from dilute aqueous solutions or purified from non-precipitable substances by salt formation and subsequent alcohol precipitation or by purification using silica get columns (QIAGEN). An advantage of the first method is, for example, the possibility of concentrating the nucleic acid during absorption in the eluate after drying. Advantages of purification using columns include the ease of handling.

Precipitation of DNA

1:10 volumes of 3 M sodium acetate (pH 5.2) and 3 volumes of 100% ethanol mixture were added to the DNA solution and stored at −20° C. for at least 4 hours. The mixture was then centrifuged for 30 min at +4° C. with 13,000×g, the supernatant was discarded and the pellet resuspended by addition of 500 μl 70% ethanol (−20° C.). After a further centrifugation step for 10 min (10 min, 13.000×g, 4° C.), the supernatant was discarded and the pellet dried for 10 min in the precooled cold trap and finally dissolved in a corresponding amount of water.

Purification of the PCR Amplificate DNA with the Qiagen PCR Purification Kit

The PCR mixture was diluted in 5 volumes of buffer PB and transferred onto the column after thorough mixing and centrifuged for 1 min. 500 μl of washing buffer PE were applied to the column and again centrifuged. The bound DNA amplificates were eluted in a clean receiver by addition of 30-50 μl water onto the column and by centrifugation.

Purification of Nucleic Acids from Agarose Gels with the Qiagen Gel Extraction Kit

The agarose gel strips with the nucleic acid to be isolated were cut out, weighed and a corresponding amount of buffer QX1 (3 volumes of the gel weight) was added. The gel was dissolved by 10 min incubation at 50° C., the solution transferred onto a column and centrifuged. After addition of 750 μl washing buffer PE and subsequent centrifugation, the bound nucleic acid was eluted into a clean receiver by addition of 30-50 μl water onto the column and subsequent centrifugation.

Isolation and Purification of RNA

RNA was extracted from tissue and cells for analysis of expression by means of RT-PCR. Extraction was carried out either by the TRIzol chloroform method with corresponding RNA isolation kits from Qiagen or Roche.

RNA Extraction Using TRIzol Reagent

During purification of RNA from tissue, the pieces of tissue (<100 mg) were weighed out and homogenized with a glass pot after addition of 1 ml TRIzol reagent. During RNA purification from cultivated cells, 1 ml TRIzol reagent for 3.5 cm² culture dish monolayer or 1 ml TRIzol reagent for centrifuged suspension culture (5-10×10⁶ cells) was applied directly to the cells and homogenized by pipetting on and off. The TRIzol mixture was incubated for approximately 5 min at ambient temperature. The mixture was shaken vigorously after addition of 20 μl chloroform, incubated for a further 5 min and finally centrifuged (20 min, 12000×g, 4° C.), so three phases could form: (a) RNA in the upper aqueous phase, (b) DNA in the middle interphase and (c) protein in the lower organic phase.

The aqueous phase was carefully removed, without touching the interphase, and reacted with 500 μl 100% isopropanol. Brief vortexing was followed by incubation for 10 min and subsequent centrifugation (10 min, 16000×g, 4° C.). The supernatant was discarded and the pellet washed with 1 ml 70% ethanol (−20° C.) and re-pelletized (10 min, 12000×g, 4° C.). The supernatant was discarded and the pellet dried in the precooled cold trap. The RNA was finally dissolved in the desired volume of water and stored at −80° C.

RNA Extraction Using Qiagen RNeasy Mini Kit

350 μl buffer RLT were added to the cell pellet (approximately 5×10⁶ cells) and the cell pellet homogenized by Qiagen shredder columns. After addition of 350 μl 70% ethanol and vigorous shaking, the suspension was applied to the column and centrifuged (30 sec at 8000×g). The column was washed by addition of 700 μl buffer RW1 and centrifugation (30 sec at 8000×g). Washing was carried out twice by addition of 500 μl buffer RPE in each case and centrifugation (30 sec, 8000×g). Washing was then carried out twice by addition of 500 μl buffer RPE and centrifugation (30 sec, 8000×g) in each case. The column was subsequently dried by centrifugation (2 min, 12000 rpm). The bound RNA was eluted by addition of 30 to 50 μl water and centrifugation (1 min at 8000×g).

DNase-I-Treatment

To avoid possible DNA contamination of the purified RNA, the RNA solution was treated enzymatically with DNase-I. For this purpose, 4 μl DNase-I and 6 μl 10× transcription buffer were added to 50 μl RNA and incubated for 30 to 45 min at 37° C. RNA purification was then carried out using QIAGEN RNeasy mini columns.

Restriction Digestion

Restriction digestion was used to check whether the desired insert is also contained in a plasmid. This may be cut out by the digestion and identified by means of its size. Restriction digestion was carried out using enzymes and reaction buffers from GincoBRL/Eggenstein and Boehringer/Mannheim. 1 μl plasmid DNA was digested in a total volume of 20 μl. The incubation time was 1 to 2 hours at a temperature corresponding to the optimum temperature for the enzyme.

Quantification of Nucleic Acids

The concentration was detected using UV spectroplatemeters. The concentration was calculated from the absorption at the specific wavelength I (for RNA, oligonucleotides and DNA 260 nm).

A=d×e×c (A absorption; d layer thickness; e material constant; c concentration). An OD260 of 1 corresponds to a concentration of: 50 μg/ml DNA, 40 μg/ml RNA or 30 μg/ml oligonucleotide. Measurement was carried out in quartz vessels which were thoroughly washed with autoclaved water between measurements.

DNA Oligonucleotide Starting Materials for siRNA Production

The oligonucleotide starting materials (ONV) were diluted in nuclease-free water to a final concentration of 200 μm, and the absorption A was measured at 260 nm of a 1:250 dilution. By means of the measured absorption, the ONV concentration B was calculated in μg/ml and B′ in μm:
A×5000=B[μg/ml]
(5000=250 fold dilution×20 μg oligo/ml/absorption unit)
B[μg/ml]/9.7=B′[μm]
(29 nt×0.333 μg/nmol for each nt=9.7 μg/nmol)

siRNA

To quantify the siRNA solutions, the absorption A was measured at 260 nm of a 1:25 dilution. The siRNA concentration B in μg/ml and B′ in μM was calculated from the measured absorption:
A×1000=B[μg/ml]
(1000=25 fold dilution×40 μg siRNA/ml/absorption unit)
B[μg/ml]/14=B′[μm]
(21 nt×2 strands=42 nt×0.333 μg/nmol for each nt=14 μg/nmol)

Quality Check by Agarose Gel Electrophoresis

1 to 2 percent TAE agarose gels with 0.5 μg/ml ethidium bromide were used to separated plasmid DNA, restriction-digested plasmid DNA, PCR products and RNA. Electrophoresis was carried out in 1× TAE buffer (cf. 2.1.3). The samples and 1 μg size label (“100 bb DNA ladder” “1 kb DNA ladder”) were reacted with ⅙ volume sample buffer and separated at 10 V/cm. Using the intercalating dye, ethidium bromide, the DNA and RNA could be made visible under UV light and documented plategraphically. To avoid degeneration during the electrophoresis of RNA, both the chamber and the comb were cleaned with ethanol and the flow buffer freshly prepared with autoclaved DEPC-H₂O.

2.5.2 Reverse Transcription Polymerase Chain Reaction (RT-PCR)

During reverse transcription, RNA is transcribed into cDNA. Oligo(dT)15-18 primers, which bind specifically to the poly-A-tail of the mRNA, for example, are used as a base for the reverse transcriptase. Depending on the starting material, 0.5-2.5 μg RNA (dissolved in 11 μl water) were mixed with 1 μl 100 nm oligo(dT) 15-18 primer and denatured for 10 min at 70° C. After cooling to 4° C., 4 μl 5× RT buffer, 2 μl 100 nM DTT, 1 μl 10 mM dNTP-Mix, 1 μl RNAsin and 1 μl Superscript™ II (200 U/μl) were added and incubation was carried out at 42° C. for 1 hours. The cDNA was stored at −20° C. until use.

2.5.3 Polymerase Chain Reaction

The polymerase chain reaction (PCR) developed in 1987 enables nucleotide sequences determined in vitro to be enzymatically copied a million times (Saiki et al., 1988). This procedure, known as amplification, also enables very small amounts of DNA to be analysed. The following reaction mixture was laid out for a PCR reaction (25 μl):

| | | | |
|---|---|---|---|
| 10x buffer | 2.5 | μl | (1x) |
| MgCl₂ (25 mM) | 3 | μl | (3 mM) |
| dNTPs (10 mM) | 0.5 | μl | (0.2 mM) |
| Primer mix (2 μM) | 5 | μl | (0.4 μM) |
| cDNA/RNA | 1 | μl | |
| Ampli - TaqPolymerase (5 U/μl) | 0.1 | μl | (0.5 U) |
| H₂O | 12.9 | μl | |

The optimum MgCl₂ concentration and the number of amplification cycles were determined individually for each primer pair (sequence, position and size of the amplicon are shown under (0)). Before the start, denaturation was carried out for 1 min at 94° C. and the duration of the individual steps of a cycle was 20 sec in each case. The thermocycler (PTC-200 (MJ Research)) was used.

Standard PCR:

| | | |
|---|---|---|
| 95° C. | 2 min | |
| 95° C. 57° C. 70° C. | 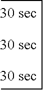 | 40x |
| 70° C. | 5 min | |
| +4° C. | ∞ | |

2.5.4 Ligation

Ligation was carried out using the T4-DNA ligase (Promega). The T4-DNA ligase is an enzyme which covalently bonds 3′- and 5′- ends of linear DNA as a function of energy (ATP). Much less energy is consumed with ‘cohesive-ended’ overhangs than with ‘blunt-ended’ overhangs. Ligation was therefore carried out for 1 hour at ambient temperature in the case of cohesive ends and overnight at 4° C. in the case of PCR products or DNA without overhangs. The pGEM-T vector was used as the plasmid.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

### BIBLIOGRAPHY

(All documents cited in the present patent application also form part of the disclosure of the present invention.)
Ahlquist, P. (2002). RNA-dependent RNA polymerases, viruses, and RNA silencing. Science 296, 1270-1273.
Aihara, Y., Mashima, H., Onda, H., Hisano, S., Kasuya, H., Hori, T., Yamada, S., Tomura, H., Yamada, Y., Inoue, I., et al. (2000). Molecular cloning of a novel brain-type Na(+)-dependent inorganic phosphate cotransporter. J Neurochem 74, 2622-2625.
Amiri, A., Keiper, B. D., Kawasaki, I., Fan, Y., Kohara, Y., Rhoads, R. E., and Strome, S. (2001). An isoform of eIF4E is a component of germ granules and is required for spermatogenesis in C. elegans. Development 128, 3899-3912.
Andres, K. H. (1961). Untersuchungen über den Feinbau von Spinalganglien. Z Zellforsch A55, 1-48.
Arriza, J. L., Eliasof, S., Kavanaugh, M. P., and Amara, S. G. (1997). Excitatory amino acid transporter 5, a retinal glutamate transporter coupled to a chloride conductance. Proc Natl Acad Sci USA 94, 4155-4160.
Arriza, J. L., Fairman, W. A., Wadiche, J. I., Murdoch, G. H., Kavanaugh, M. P., and Amara, S. G. (1994). Functional comparisons of three glutamate transporter subtypes cloned from human motor cortex. J Neurosci 14, 5559-5569.
Baccei, M. L., Bardoni, R., and Fitzgerald, M. (2003). Development of nociceptive synaptic inputs to the neonatal rat dorsal horn: glutamate release by capsaicin and menthol. J Physiol 549, 231-242.
Bai,-L., Xu, H., Collins, J. F., and Ghishan, F. K. (2001). Molecular and functional analysis of a novel neuronal vesicular glutamate transporter. J Biol Chem 276, 36764-36769.
Bai, L., Zhang, X., and Ghishan, F. K. (2003). Characterization of vesicular glutamate transporter in pancreatic alpha- and beta-cells and its regulation by glucose. Am J Physiol Gastrointest Liver Physiol 284, G808-814.
Banerjea, A., Li, M. J., Bauer, G., Remling, L., Lee, N. S., Rossi, J., and Akkina, R. (2003). Inhibition of HIV-1 by lentiviral vector-transduced siRNAs in T lymphocytes differentiated in SCID-hu mice and CD34+ progenitor cell-derived macrophages. Mol Ther 8, 62-71.
Baranauskas, G., and Nistri, A. (1998). Sensitization of pain pathways in the spinal cord: cellular mechanisms. Prog Neurobiol 54, 349-365.
Barton, G. M., and Medzhitov, R. (2002). Retroviral delivery of small interfering RNA into primary cells. Proc Natl Acad Sci USA 99, 14943-14945.
Bass, B. L. (2000). Double-stranded RNA as a template for gene silencing. Cell 101, 235-238.
Bellocchio, E. E., Hu, H., Pohorille, A., Chan, J., Pickel, V. M., and Edwards, R. H. (1998). The localization of the brain-specific inorganic phosphate transporter suggests a specific presynaptic role in glutamatergic transmission. J Neurosci 18, 8648-8659.
Bellocchio, E. E., Reimer, R. J., Fremeau, R. T., Jr., and Edwards, R. H. (2000). Uptake of glutamate into synaptic vesicles by an inorganic phosphate transporter. Science 289, 957-960.
Bender, A. S., Reichelt, W., and Norenberg, M. D. (2002a). Characterization of cystine uptake in cultured astrocytes. Neurochem Int 37, 269-276.
Bender, F. L. P., Mederos y Schnitzler, M., Li, Y., Ji, A., Weihe, E., Gudermann, T., and Schafer, M. K.-H. (2002b). Expression and functional characterization of the vanilloid receptor-like TRP channel VRL-1 in the primary sensory cell line F-11. 2002 Abstract Viewer Itinerary Planner Program No. 48.23.
Bernstein, E., Caudy, A. A., Hammond, S. M., and Hannon, G. J. (2001). Role for a bidentate ribonuclease in the initiation step of RNA interference. Nature 409, 363-366.
Boland, L. M., and Dingledine, R. (1990). Expression of sensory neuron antigens by a dorsal root ganglion cell line, F-11. Brain Res Dev Brain Res 51, 259-266.
Bothwell, M. A., Schechter, A. L., and Vaughn, K. M. (1980). Clonal variants of PC12 pheochromocytoma cells with altered response to nerve growth factor. Cell 21, 857-866.
Brummelkamp, T. R., Bernards, R., and Agami, R. (2002). A system for stable expression of short interfering RNAs in mammalian cells. Science 296, 550-553.
Burger, P. M., Mehl, E., Cameron, P. L., Maycox, P. R., Baumert, M., Lottspeich, F., De Camilli, P., and Jahn, R. (1989). Synaptic vesicles immunoisolated from rat cerebral cortex contain high level of glutamate. Neuron 7, 287-293.
Bushman, F. (2003). RNA interference: applications in vertebrates. Mol Ther 7, 9-10.
Calderon-Martinez, D., Garavito, Z., Spinel, C., and Hurtado, H. (2002). Schwann cell-enriched cultures from adult human peripheral nerve: a technique combining short enzymatic dissociation and treatment with cytosine arabinoside (Ara-C). J Neurosci Methods 114, 1-8.
Carlton, S. M., Zhou, S., and Coggeshall, R. E. (1998). Evidence for the interaction of glutamate and NK1 receptors in the periphery. Brain Res 790, 160-169.
Carrigan, C. N., Bartlett, R. D., Esslinger, C. S., Cybulski, K. A., Tongcharoensirikul, P., Bridges, R. J., and Thompson, C. M. (2002). Synthesis and in vitro pharmacology of substituted quinoline-2,4-dicarboxylic acids as inhibitors of vesicular glutamate transport. J Med Chem 45, 2260-2276.
Casey, B. P., and Glazer, P. M. (2001). Gene targeting via triple-helix formation. Prog Nucleic Acid Res Mol Biol 67, 163-192.
Caterina, M. J., Rosen, T. A., Tominaga, M., Brake, A. J., and Julius, D. (1999). A capsaicin-receptor homologue with a high threshold for noxious heat. Nature 398, 436-441.
Caterina, M. J., Schumacher, M. A., Tominaga, M., Rosen, T. A., Levine, J. D., and Julius, D. (1997). The capsaicin receptor: a heat-activated ion channel in the pain pathway. Nature 389, 816-824.
Chen, L., and Huang, M. L.-Y. (1992). Protein kinase C reduces Mg2+ block of NMDA-receptor channels as a mechanism of modulation. Nature 356, 521-523.
Chong, S. S., Kristjansson, K., Zoghbi, H. Y., and Hughes, M. R. (1993). Molecular cloning of the cDNA encoding a human renal sodium phosphate transport protein and its assignment to chromosome 6p21.3-p23. Genomics 18, 355-359.
Coburn, G. A., and Cullen, B. R. (2002). Potent and specific inhibition of human immunodeficiency virus type 1 replication by RNA interference. J Virol 76, 9225-9231.
Coonrod, A., Li, F. Q., and Horwitz, M. (1997). On the mechanism of DNA transfection: efficient gene transfer without viruses. Gene Ther 4, 1313-1321.
Davis, J. B., Gray, J., Gunthorpe, M. J., Hatcher, J. P., Davey, P. T., Overend, P., Harries, M. H., Latcham, J., Clapham, C., Atkinson, K., et al. (2000). Vanilloid receptor-1 is essential for inflammatory thermal hyperalgesia. Nature 405, 183-187.
Devroe, E., and Silver, P. A. (2002). Retrovirus-delivered siRNA. BMC Biotechnol 2, 15.
Disbrow, J., Gershten, M., and Ruth, J. (1982). Uptake of L-[3H]glutamic acid by crude and purified synaptic vesicles from rat brain. Biochem Biophys Res Commun 108, 1221-1227.
Doench, J. G., Petersen, C. P., and Sharp, P. A. (2003). siRNAs can function as miRNAs. Genes Dev 17, 438-442.
Dykxhoorn, D. M., Novina, C. D., and Sharp, P. A. (2003). Killing the messenger: Short RNAs that silence gene expression. Nat Rev Mol Cell Biol 4, 457-467.
Eggert, C., and Fischer, U. (2003). RNA-lnterferenz: Ein Werkzeug zur Analyse der Genfunktion. BIOspektrum 9, 372-274.
Elbashir, S. M., Harborth, J., Lendeckel, W., Yalcin, A., Weber, K., and Tuschl, T. (2001a). Duplexes of 21-nucleotide RNAs mediate RNA interference in cultured mammalian cells. Nature 411, 494-498.
Elbashir, S. M., Harborth, J., Weber, K., and Tuschl, T. (2002). Analysis of gene function in somatic mammalian cells using small interfering RNAs. Methods 26, 199-213.
Elbashir, S. M., Martinez, J., Patkaniowska, A., Lendeckel, W., and Tuschl, T. (2001b). Functional anatomy of siRNAs for mediating efficient RNAi in Drosophila melanogaster embryo lysate. Embo J 20, 6877-6888.
Erdreich-Epstein, A., and Shackleford, G. M. (1998). Differential expression of Wnt genes in normal and flat variants of PC12 cells, a cell line responsive to ectopic Wnt1 expression. Growth Factors 15, 149-158.
Fagg, G. E., and Foster, A. C. (1983). Amino acid neurotransmitters and their pathways in the mammalian brain. Neuroscience 9, 701-719.
Fairman, W. A., Vandenberg, R. J., Arriza, J. L., Kavanaugh, M. P., and Amara, S. G. (1995). An excitatory amino-acid transporter with properties of a ligend-gated chloride channel. Nature 375, 599-603.
Felgner, P. L., Gadek, T. R., Holm, M., Roman, R., Chan, H. W., Wenz, M., Northrop, J. P., Ringold, G. M., and Danielsen, M. (1987). Lipofection: a highly efficient, lipid-mediated DNA-transfection procedure. Proc Natl Acad Sci USA 84, 7413-7417.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-811.
Fonnum, F. (1984). Glutamate: A neurotransmitter in the mammalian brain. J Neurochem 42.
Forth, W., Henschler, D., and Rummel, W. (2001). Allgemeine und spezielle Pharmakologie und Toxikologie, 8 edn (München—Jena, Urban & Fischer Verlag).
Francel, P. C., Harris, K., Smith, M., Fishman, M. C., Dawson, G., and Miller, R. J. (1987). Neurochemical characteristics of a novel dorsal root ganglion X neuroblastoma hybrid cell line, F-11. J Neurochem 48, 1624-1631.
Fremeau, R. T., Jr., Burman, J., Qureshi, T., Tran, C. H., Proctor, J., Johnson, J., Zhang, H., Sulzer, D., Copenhagen, D. R., Storm-Mathisen, J., et al. (2002). The identification of vesicular glutamate transporter 3 suggests novel modes of signaling by glutamate. Proc Natl Acad Sci USA 99, 14488-14493.
Fremeau, R. T., Jr., Troyer, M. D., Pahner, I., Nygaard, G. O., Tran, C. H., Reimer, R. J., Bellocchio, E. E., Fortin, D., Storm-Mathisen, J., and Edwards, R. H. (2001). The expression of vesicular glutamate transporters defines two classes of excitatory synapse. Neuron 31, 247-260.
Gareis, M., Harrer, P., and Bertling, W. M. (1991). Homologous recombination of exogenous DNA fragments with genomic DNA in somatic cells of mice. Cell Mol Biol 37, 191-203.
Gasnier, B. (2000). The loading of neurotransmitters into synaptic vesicles. Biochimie 82, 327-337.
Gershon, H., Ghirlando, R., Guttman, S. B., and Minsky, A. (1993). Mode of formation and structural features of DNA-cationic liposome complexes used for transfection. Biochemistry 32, 7143-7151.
Gil, J., and Esteban, M. (2000). Induction of apoptosis by the dsRNA-dependent protein kinase (PKR): mechanisms of action. Apoptosis 5.
Gitlin, L., Karelsky, S., and Andino, R. (2002). Short interfering RNA confers intracellular antiviral immunity in human cells. Nature 418, 430-434.
Gonzalez, M. P., Herrero, M. T., Vicente, S., and Oset-Gasque, M. J. (1998). Effect of glutamate receptor agonists on catecholamine secretion in bovine chromaffin cells. Neuroendocrinology 67, 181-189.
Gras, C., Herzog, E., Bellenchi, G. C., Bernard, V., Ravassard, P., Pohl, M., Gasnier, B., Giros, B., and El Mestikawy, S. (2002). A third vesicular glutamate transporter expressed by cholinergic and serotoninergic neurons. J Neurosci 22, 5442-5451.
Greene, G. C. (1949). Gross anatomy in the rat in laboratory investigation. (Philadelphia, J. B. Lippincott Company).
Greene, L. A., and Rein, G. (1977). Release, storage and uptake of catecholamines by a clonal cell line of nerve growth factor responsive pheo-chromocytoma cells. Brain Res 129, 247-263.
Greene, L. A., and Tischler, A. S. (1976). Establishment of a noradrenergic clonal line of rat adrenal pheochromocytoma cells which respond to nerve growth factor. Proc Natl Acad Sci USA 73, 2424-2428.
Grothe, C., and Unsicker, K. (1987). Neuron-enriched cultures of adult rat dorsal root ganglia: establishment, characterization, survival, and neuropeptide expression in response to trophic factors. J Neurosci Res 18, 539-550.
Grunweller, A., Wyszko, E., Bieber, B., Jahnel, R., Erdmann, V. A., and Kurreck, J. (2003). Comparison of different antisense strategies in mammalian cells using locked nucleic acids, 2′-O-methyl RNA, phosphorothioates and small interfering RNA. Nucleic Acids Res 31, 3185-3193.
Grynfeld, A., Pahlman, S., and Axelson, H. (2000). Induced neuroblastoma cell differentiation, associated with transient HES-1 activity and reduced HASH-1 expression, is inhibited by Notch1. Int J Cancer 88, 401-410.
Guerrero, I., Pellicer, A., and Burstein, D. E. (1988). Dissociation of c-fos from ODC expression and neuronal differentiation in a PC12 subline stably transfected with an inducible N-ras oncogene. Biochem Biophys Res Commun 150, 1185-1192.
Guerrero, I., Wong, H., Pellicer, A., and Burstein, D. E. (1986). Activated N-ras gene induces neuronal differentiation of PC12 rat pheochromocytoma cells. J Cell Physiol 129, 71-76.
Guo, S., and Kemphues, K. J. (1995). par-1, a gene required for establishing polarity in C. elegans embryos, encodes a putative Ser/Thr kinase that is asymmetrically distributed. Cell 81, 611-620.
Hammond, S. M., Bernstein, E., Beach, D., and Hannon, G. J. (2000). An RNA-directed nuclease mediates posttranscriptional gene silencing in Drosophila cells. Nature 404, 293-296.
Hayashi, M., Otsuka, M., Morimoto, R., Hirota, S., Yatsushiro, S., Takeda, J., Yamamoto, A., and Moriyama, Y. (2001). Differentiation-associated Na+-dependent inorganic phosphate cotransporter (DNPI) is a vesicular glutamate transporter in endocrine glutamatergic systems. J Biol Chem 276, 43400-43406.
Hayashi, M., Yamada, H., Uehara, S., Morimoto, R., Muroyama, A., Yatsushiro, S., Takeda, J., Yamamoto, A., and Moriyama, Y. (2003). Secretory granule-mediated co-secretion of L-glutamate and glucagon triggers glutamatergic signal transmission in islets of Langerhans. J Biol Chem 278, 1966-1974.
Hemann, M. T., Fridman, J. S., Zilfou, J. T., Hernando, E., Paddison, P. J., Cordon-Cardo, C., Hannon, G. J., and Lowe, S. W. (2003). An epi-allelic series of p53 hypomorphs created by stable RNAi produces distinct tumor phenotypes in vivo. Nat Genet 33, 396-400.
Herzog, E., Bellenchi, G. C., Gras, C., Bernard, V., Ravassard, P., Bedet, C., Gasnier, B., Giros, B., and El Mestikawy, S. (2001). The existence of a second vesicular glutamate transporter specifies subpopulations of glutamatergic neurons. J Neurosci 21, RC181.
Hioki, H., Fujiyama, F., Taki, K., Tomioka, R., Furuta, T., Tamamaki, N., and Kaneko, T. (2003). Differential distribution of vesicular glutamate transporters in the rat cerebellar cortex. Neuroscience 117, 1-6.
Hisano, S., Sawada, K., Kawano, M., Kanemoto, M., Xiong, G., Mogi, K., Sakata-Haga, H., Takeda, J., Fukui, Y., and Nogami, H. (2002). Expression of inorganic phosphate/vesicular glutamate transporters (BNPI/VGLUT1 and DNPI/VGLUT2) in the cerebellum and precerebellar nuclei of the rat. Brain Res Mol Brain Res 107, 23-31.
Holen, T., Amarzguioui, M., Wiiger, M. T., Babaie, E., and Prydz, H. (2002). Positional effects of short interfering RNAs targeting the human coagulation trigger Tissue Factor. Nucleic Acids Res 30, 1757-1766.
Hu, W. Y., Myers, C. P., Kilzer, J. M., Pfaff, S. L., and Bushman, F. D. (2002). Inhibition of retroviral pathogenesis by RNA interference. Curr Biol 12, 1301-1311.
Huber, K., Brühl, B., Guillemont, F., Olson, E. N., Ernsberger, U., and Unsicker, K. (2002a). Development of chromaffin cells depends on MASH1 function. Development 129, 4729-4738.
Huber, K., Combs, S., Ernsberger, U., Kalcheim, C., and Unsicker, K. (2002b). Generation of neuroendocrine chromaffin cells from sympathoadrenal preogenitors: beyond the glucocorticoid hypothesis. Ann N Y Acad Sci 971, 554-549.
Hunt, S. P., and Mantyh, P. W. (2001). The molecular dynamics of pain control. Nat Rev Neuroscience 2, 83-91.
Israel, M., Tomasi, M., Bostel, S., and Meunier, F. M. (2001). Cellular resistance to Evans blue toxicity involves an up-regulation of a phosphate transporter implicated in vesicular glutamate storage. J Neurochem 78, 658-663.
Issack, P. S., and Ziff, E. B. (1998a). Altered expression of helix-loop-helix transcriptional regulators and cyclin D1 in Wnt-1 -transformed PC12 cells. Cell Growth Differ 9, 837-845.
Issack, P. S., and Ziff, E. B. (1998b). Genetic elements regulating HES-1 induction in Wnt-1-transformed PC12 cells. Cell Growth Differ 9, 827-836.
Jacque, J. M., Triques, K., and Stevenson, M. (2002). Modulation of HIV-1 replication by RNA interference. Nature 418, 435-438.
Jahn, R., and Südhof, T. C. (1993). Synaptic vesicle traffic: rush hour in the nerve terminal. J Neurochem 61, 12-21.
Jimenez, A. L., Chou, A. H., Khadadadi, O., Palos, T. P., and Howard, B. D. (2003). Wnt-1 has multiple effects on the expression of glutamate transporters. Neurochem Int 42, 345-351.
Jogi, A., Persson, P., Grynfeld, A., Pahlman, S., and Axelson, H. (2002). Modulation of basic helix-loop-helix transcription complex formation by Id proteins during neuronal differentiation. J Biol Chem 277, 9118-9126.
Jue, S. F., Bradley, R. S., Rudnicki, J. A., Varmus, H. E., and Brown, A. M. (1992). The mouse Wnt-1 gene can act via paracrine mechanism in transformation of mammary epithelial cells. Mol Cell Biol 12, 321-328.
Kamath, R. S., Fraser, A. G., Dong, Y., Poulin, G., Durbin, R., Gotta, M., Kanapin, A., Le Bot, N., Moreno, S., Sohrmann, M., et al. (2003). Systematic functional analysis of the Caenorhabditis elegans genome using RNAi. Nature 421, 231-237.
Kanai, Y., and Hediger, M. A. (1992). Primary structure and functional characterization of a high-affinity glutamate transporter. Nature 360, 467-471.
Kaneko, T., and Fujiyama, F. (2002). Complementary distribution of vesicular glutamate transporters in the central nervous system. Neurosci Res 42, 243-250.
Kapadia, S. B., Brideau-Andersen, A., and Chisari, F. V. (2003). Interference of hepatitis C virus RNA replication by short interfering RNAs. Proc Natl Acad Sci USA 100, 2014-2018.
Katoh-Semba, R., Oohira, A., Sano, M., Watanabe, K., Kitajima, S., and Kashiwamata, S. (1989). Glycosaminoglycan composition of PC12 pheochromocytoma cells: a comparison with PC12D cells, a new subline of PC12 cells. J Neurochem 52, 889-895.
Keck, J. L., Roche, D. D. Lynch, A. S., and Berger, J. M. (2000). Structure of the RNA polymerase domain of E. coli primase. Science 287; 2482-2486.
Kennerdell, J. R., and Carthew, R. W. (2000). Heritable gene silencing in Drosophila using double-stranded RNA. Nat Biotechnol 18, 896-898.
Kish, P. E., Fischer-Bovenkerk, C., and Ueda, T. (1989). Active transport of GABA and glycin into synaptic vesicles. Proc Natl Acad Sci USA 86, 3877-3881.
Kobayashi, S., and Millhorn, D. E. (2001). Hypoxia regulates glutamate metabolism and membrane transport in rat PC12 cells. J Neurochem 76, 1935-1948.
Lee, N. S., Dohjima, T., Bauer, G., Li, H., Li, M. J., Ehsani, A., Salvaterra, P., and Rossi, J. (2002). Expression of small interfering RNAs targeted against HIV-1 rev transcripts in human cells. Nat Biotechnol 20, 500-505.
Lee, R. Y., Sawin, E. R., Chalfie, M., Horvitz, H. R., and Avery, L. (1999). EAT-4, a homolog of a mammalian sodium-dependent inorganic phosphate cotransporter, is necessary for glutamatergic neurotransmission in caenorhabditis elegans. J Neurosci 19, 159-167.
Levy, L. M., Warr, O., and Attwell, D. (1998). Stoichiometry of the glial glutamate transporter GLT-1 expressed inducibly in a Chinese hamster ovary cell line selected for low endogenous Na+-dependent glutamate uptake. J Neurosci 18, 9620-9628.
Li, H., Li, W. X., and Ding, S. W. (2002). Induction and suppression of RNA silencing by an animal virus. Science 296, 1319-1321.
Li, J. L., Fujiyama, F., Kaneko, T., and Mizuno, N. (2003a). Expression of vesicular glutamate transporters, VGluT1 and VGluT2, in axon terminals of nociceptive primary afferent fibers in the superficial layers of the medullary and spinal dorsal horns of the rat. J Comp Neurol 457, 236-249.
Li, J. L., Xiong, K. H., Dong, Y. L., Fujiyama, F., Kaneko, T., and Mizuno, N. (2003b). Vesicular glutamate transporters, VGluT1 and VGluT2, in the trigeminal ganglion neurons of the rat, with special reference to coexpression. J Comp Neurol 463, 212-220.
Lindbo, J. A., and Dougherty, W. G. (1992). Untranslatable transcripts of the tobacco etch virus coat protein gene sequence can interfere with tobacco etch virus replication in transgenic plants and protoplasts. Virology 189, 725-733.
Luqmani, Y. A. (1981). Nucleotide uptake by isolated cholinergic synaptic vesicles: Evidence for a carrier of adenosine 5′-triphosphate. Neuroscience 6, 1011-1021.
Mackenzie, B., and Erickson, J. (2003). Sodium-coupled neutral amino acid (System N/A) transporters of the SLC38 family. Pflugers Arch.
Macnaughton, T. B., Shi, S. T., Modahl, L. E., and Lai, M. M. (2002). Rolling circle replication of hepatitis delta virus RNA is carried out by two different cellular RNA polymerases. J Virol 76, 3920-3927.
Manfras, B. J., and Boehm, B. O. (1995). Expression of a glutamate transporter cDNA in human pancreatic islets. Exp Clin Endocrinol Diabetes 103 Suppl 2, 95-98.
Masson, J., Sagne, C., Hamon, M., and El Mestikawy, S. (1999). Neurotransmitter transporters in the central nervous system. Pharmacol Rev 51, 439-464.
McIntire, S. L., Reimer, R. J., Schuske, K., Edwards, R. H., and Jorgensen, E. M. (1997). Identification and characterization of the vesicular GABA transporter. Nature 389, 870-876.
McMahon, A. P., and Bradley, A. (1990). The Wnt-1 (Int-1) proto-oncogen is required for development of brain. Cell 62, 1071-1085.
McManus, M. T., and Sharp, P. A. (2002). Gene silencing in mammals by small interfering RNAs. Nat Rev Genet 3, 737-747.
Mertz, K. D., Weisheit, G., Schilling, K., and Luers, G. H. (2002). Electroporation of primary neural cultures: a simple method for directed gene transfer in vitro. Histochem Cell Biol 118, 501-506.
Miyagishi, M., and Taira, K. (2002). U6 promoter-driven siRNAs with four uridine 3′ overhangs efficiently suppress targeted gene expression in mammalian cells. Nat Biotechnol 20, 497-500.
Moraleda, G., and Taylor, J. (2001). Host RNA polymerase requirements for transcription of the human hepatitis delta virus genome. J Virol 75, 10161-10169.
Myslinski, E. (2001). An unusually compact external promotor for RNA polymerase III transcription of the human H1RNA gene. Nucleic Acids Res 29, 2502-2509.
Naito, S., and Ueda, T. (1985). Characterization of glutamate uptake into synaptic vesicles. J Neurochem 44, 99-109.
Napoli, C., Lemieux, C., and Jorgensen, R. (1990). Introduction of a Chimeric Chalcone Synthase Gene into Petunia Results in Reversible Co-Suppression of Homologous Genes in trans. Plant Cell 2, 279-289.
Nestler, E. J., Hyman, S. E., and Malenka, R. C. (2001). Molecular Neuropharmacology, 1 edn, McGraw-Hill Companies).
Ni, B., Du, Y., Wu, X., DeHoff, B. S., Rosteck, P. R., Jr., and Paul, S. M. (1996). Molecular cloning, expression, and chromosomal localization of a human brain-specific Na(+)-dependent inorganic phosphate cotransporter. J Neurochem 66, 2227-2238.
Ni, B., Rosteck, P. R., Jr., Nadi, N. S., and Paul, S. M. (1994). Cloning and expression of a cDNA encoding a brain-specific Na(+)-dependent inorganic phosphate cotransporter. Proc Natl Acad Sci USA 91, 5607-5611.
Ni, B., Wu, X., Yan, G. M., Wang, J., and Paul, S. M. (1995). Regional expression and cellular localization of the Na(+)-dependent inorganic phosphate cotransporter of rat brain. J Neurosci 15, 5789-5799.
Njus, D., Kelley, P. M., and Hardabek, G. J. (1986). Bioenegetics of secretory vesicles. Biochim Biophys Acta 853, 237-265.
Noda, M., Ko, M., Ogura, A., Liu, D. G., Amano, T., Takano, T., and Ikawa, Y. (1985). Sarcoma viruses carrying ras oncogenes induce differentiation-associated properties in a neuronal cell line. Nature 318, 73-75.
Novina, C. D., Murray, M. F., Dykxhoorn, D. M., Beresford, P. J., Riess, J., Lee, S. K., Collman, R. G., Lieberman, J., Shankar, P., and Sharp, P. A. (2002). siRNA-directed inhibition of HIV-1 infection. Nat Med 8, 681-686.
Oliveira, A. C. R., Hydling, F., Olsson, E., Shi, T., Edwards, R. H., Fujiyama, F., Kaneko, T., Hökfelt, T., Cullheim, S., and Meister, B. (2003). Cellular Localization of Three Vesikular Glutamate Transporter mRNAs and Proteins in Rat Spinal Cord and Dorsal Root Ganglia. Synapse 50, 117-129.
Oset-Gasque, M. J., Castro, E., and Gonzalez, A. M. (1990). Mechanisms of 3H-GABA release by chromaffin cells in primary culture. J Neurosci Res 26, 181-187.
Ottersen, O. P., and Storm-Mathisen, J. (1984). Neurons containing or accumulating transmitter amino acids. In Handbook of Chemical Neuroanatomy, A. Björklund, H. T., and M. J. Kuhar, eds. (Amsterdam, Elsevier), pp. 141-246.
Ozkan, E. D., and Ueda, T. (1998). Glutamate transport and storage in synaptic vesicles. Jpn J Pharmacol 77, 1-10.
Paddison, P. J., and Hannon, G. J. (2002). RNA interference: the new somatic cell genetics? Cancer Cell 2, 17-23.
Palauqui, J. C. e. a. (1997). Systemic acquired silencing: transgenespecific post-transcriptional silencing is transmitted by grafting from silenced stocks to non-silenced scions. Embo J 16, 4738-4745.
Patapoutian, A., Peier, A. M., Story, G. M., and Viswanath, V. (2003). ThermoTRP channnels and beyond: mechanisms of temperature sensation. Nat Rev Neuroscience 4, 529-539.
Patapoutian, A., and Reichardt, L. F. (2001). Trk receptors: mediators of neurotrophin action. Curr Opin Neurobiol 11, 272-280.
Pines, G., Danbolt, N. C., Bjoras, M., Zhang, Y., Bendahan, A., Eide, L., Koepsell, H., Storm-Mathisen, J., Seeberg, E., and Kanner, B. I. (1992). Cloning and expression of a rat brain L-glutamate transporter. Nature 360, 464-467.
Plasterk, R. H. (2002). RNA silencing: the genome's immune system. Science 296, 1263-1265.
Platika, D., Baizer, L., and Fishman, M. C. (1985). Sensory neurons “immortalized” by fusion with neuroblastoma cells. Trans Assoc Am Physicians 98, 301-304.
Qin, X. F., An, D. S., Chen, I. S., and Baltimore, D. (2003). Inhibiting HIV-1 infection in human T cells by lentiviral-mediated delivery of small interfering RNA against CCR5. Proc Natl Acad Sci USA 100, 183-188.
Ramachandran, B., Houben, K., Rozenberg, Y. Y., Haigh, J. R., Varpetian, A., and Howard, B. D. (1993). Differential expression of transporters for norepinephrine and glutamate in wild type, variant, and WNT1-expressing PC12 cells. J Biol Chem 268, 23891-23897.
Reimer, R. J., and Edwards, R. H. (2003). Organic anion transport is the primary function of the SLC17/type I phosphate transporter family. Pflugers Arch.
Ren, K. (1994). Wind up and the NMDA-receptor: from animal studies to humans. Pain 59, 157-158.
Romero, O., Figueroa, S., Vicente, S., Gonzalez, M. P., and Oset-Gasque, M. J. (2003). Molecular mechanisms of glutamate release by bovine chromaffin cells in primary culture. Neuroscience 116, 817-829.
Roseth, S., Fykse, E. M., and Fonnum, F. (1995). Uptake of L-glutamate into rat brain synaptic vesicles: effect of inhibitors that bind specifically to the glutamate transporter. J Neurochem 65, 96-103.
Roseth, S., Fykse, E. M., and Fonnum, F. (1998). Uptake of L-glutamate into synaptic vesicles: competitive inhibition by dyes with biphenyl and amino- and sulphonic acid-substituted naphthyl groups. Biochem Pharmacol 56, 1243-1249.
Rothstein, J. D., Dykes-Hoberg, M., Pardo, C. A., Bristol, L. A., Jin, L., Kuncl, R. W., Kanai, Y., Hediger, M. A., Wang, Y., Schielke, J. P., and Welty, D. F. (1996). Knockout of glutamate transporters reveals a major role for astroglial transport in excitotoxicity and clearance of glutamate. Neuron 16, 675-686.
Rozenberg, Y. Y., and Howard, B. D. (1994). Contrasting morphological changes in PC12 flat cells expressing two different forms of exogenous encogenic ras. Exp Cell Res 211, 59-67.
Rubinson, D. A., Dillon, C. P., Kwiatkowski, A. V., Sievers, C., Yang, L., Kopinja, J., Rooney, D. L., Ihrig, M. M., McManus, M. T., Gertler, F. B., et al. (2003). A lentivirusbased system to functionally silence genes in primary mammalian cells, stem cells and transgenic mice by RNA interference. Nat Genet 33, 401-406.
Sagné, C., El Mestikawy, S., Isambert, M.-F., Hamon, M., Henry, J.-P., Giros, B., and Gasnier, B. (1997). Cloning of a functional GABA and glycine transporter by screening of genome databases. FEBS Lett 417, 177-183.
Saiki, R. K., Gelfand, D. H., Stoffel, S., Scharf, S. J., Higuchi, R., Horn, G. T., Mullis, K. B., and Erlich, H. A. (1988). Primer-directed enzymatic amplification of DNA with a thermostable DNA polymerase. Science 239, 487-491.
Sassone-Corsi, P., Der, C. J., and Verma, I. M. (1989). ras-induced differentiation of PC12 cells: possible involment of fos and jun. Mol Cell Biol 9, 3174-3183.
Schafer, M. K., Varoqui, H., Defamie, N., Weihe, E., and Erickson, J. D. (2002). Molecular cloning and functional identification of mouse vesicular glutamate transporter 3 and its expression in subsets of novel excitatory neurons. J Biol Chem 277, 50734-50748.
Scholz, J., and Woolf, C. J. (2002). Can we conquer pain? Nat Neurosci 5, 1062-1067.
Schwarz, D. S., Hutvagner, G., Haley, B., and Zamore, P. D. (2002). Evidence that siRNAs function as guides, not primers, in the Drosophila and human RNAi pathways. Mol Cell 10, 537-548.
Shackleford, G. M., Willert, K., Wang, J., and Varmus, H. E. (1993). The Wnt-1 proto-oncogene induces changes in morphology, gene expression and growth factor responsivness in PC12 cells. Neuron 11, 865-875.
Shen, C., Buck, A. K., Liu, X., Winkler, M., and Reske, S. N. (2003). Gene silencing by adenovirus-delivered siRNA. FEBS Lett 539, 111-114.
Shi, Y. (2003). Mammalian RNAi for the masses. Trends Genet 19, 9-12.
Shioi, J., Naito, S., and Ueda, T. (1989). Glutamate uptake into synaptic vesicles from bovine cerebral cortex and electrochemical potential difference of proton across the membrane. Biochem J 258, 449-504.
Shuey, D. J., McCallus, D. E., and Giordano, T. (2002). RNAi: gene-silencing in therapeutic intervention. Drug Discov Today 7, 1040-1046.
Sijen, T., Fleenor, J., Simmer, F., Thijssen, K. L., Parrish, S., Timmons, L., Plasterk, R. H., and Fire, A. (2001). On the role of RNA amplification in dsRNA-triggered gene silencing. Cell 107, 465-476.
Sindrup, S. H., and Jensen, T. S. (1999). Efficiacy of pharmacological treatments of neuropathic pain: an update and effect related to mechanism of drug action. Pain 83, 389-400.
Skerry, T. M., and Genever, P. G. (2001). Glutamate signalling in non-neuronal tissues. Trends Pharmacol Sci 22, 174-181.
Smith, J. G., Walzem, R. L., and German, J. B. (1993). Liposomes as agents of DNA transfer. Biochim Biophys Acta 1154, 327-340.
Snider, W. D., and McMahon, S. B. (1998). Tackling pain at the source: new ideas about nociceptors. Neuron 20, 629-632.
Stark, G. (1998). How cells respond to interferons. Annu Rev Biochem 67, 227-264.
Stewart, S. A., Dykxhoorn, D. M., Palliser, D., Mizuno, H., Yu, E. Y., An, D. S., Sabatini, D. M., Chen, I. S., Hahn, W. C., Sharp, P. A., et al. (2003). Lentivirus-delivered stable gene silencing by RNAi in primary cells. Rna 9, 493-501.
Storck, T., Schulte, S., Hofmann, K., and Stoffel, W. (1992). Structure, expression, and functional analysis of a Na(+)-dependent glutamate/aspartate transporter from rat brain. Proc Natl Acad Sci USA 89, 10955-10959.
Stucky, C. L., and Lewin, G. R. (1999). Isolectin B(4)-positive and -negative nociceptors are functionally distinct. J Neurosci 19, 6497-6505.
Sung, B., Lim, G., and Mao, J. (2003). Altered expression and uptake activity of spinal glutamate transporters after nerve injury contribute to the pathogenesis of neuropathic pain in rats. J Neurosci 23, 2899-2910.
Tabb, J., Kish, P., Vandyke, R., and Ueda, T. (1992). Glutamate transport into synaptic vesicles—role of membrane potential, pH gradient and intravesicular pH. J Biol Chem 267, 15412-15418.
Takamori, S., Malherbe, P., Broger, C., and Jahn, R. (2002). Molecular cloning and functional characterization of human vesicular glutamate transporter 3. EMBO Rep 3, 798-803.
Takamori, S., Rhee, J. S., Rosenmund, C., and Jahn, R. (2000). Identification of a vesicular glutamate transporter that defines a glutamatergic phenotype in neurons. Nature 407, 189-194.
Tijsterman, M., Ketting, R. F., Okihara, K. L., Sijen, T., and Plasterk, R. H. (2002). RNA helicase MUT-14-dependent gene silencing triggered in C. elegans by short antisense RNAs. Science 295, 694-697.
Timmons, L., Court, D. L., and Fire, A. (2001). Ingestion of bacterially expressed dsRNAs can produce specific and potent genetic interference in Caenorhabditis elegans. Gene 263, 103-112.
Todd, A. J., Hughes, D. I., Polgar, E., Nagy, G. G., Mackie, M., Ottersen, O. P., and Maxwell, D. J. (2003). The expression of vesicular glutamate transporters VGLUT1 and VGLUT2 in neurochemically defined axonal populations in the rat spinal cord with emphasis on the dorsal horn. Eur J Neurosci 17, 13-27.
Toll, L., and Howard, D. B. (1980). Evidence that an ATPase and a proton-motive force function in the transport of acetylcholine. J Biol Chem 255, 1787-1789.
Tölle, T. R. (1997). Chronischer Schmerz. In Klinische Neurobiologie: molekulare Pathogenese und Therapie von neurologischen Erkrankungen, T. Herdegen, T. R. Tölle, and M. Bähr, eds. (Heidelberg, Spektrum Akademischer Verlag), pp. 307-336.
Tominaga, M., Caterina, M. J., Malmberg, A. B., Rosen, T. A., Gilbert, H., Skinner, K., Raumann, B. E., Basbaum, A. I., and Julius, D. (1998). The cloned capsaicin receptor integrates multiple pain-producing stimuli. Neuron 21, 531-543.
Tuschl, T. (2002). Expanding small RNA interference. Nat Biotechnol 20, 446-448.
Vaistij, F. E., Jones, L., and Baulcombe, D. C. (2002). Spreading of RNA targeting and DNA methylation in RNA silencing requires transcription of the target gene and a putative RNA-dependent RNA polymerase. Plant Cell 14, 857-867.
Vandenberg, R. J. (1998). Molecular pharmacology and physiology of glutamate transporters in the central nervous system. Clin Exp Pharmacol Physiol 25, 393-400.
Varoqui, H., Schafer, M. K., Zhu, H., Weihe, E., and Erickson, J. D. (2002). Identification of the differentiation-associated Na+/PI transporter as a novel vesicular glutamate transporter expressed in a distinct set of glutamatergic synapses. J Neurosci 22, 142-155.
Wall, P. D., and Melzack, R. (1999). Textbook of pain (Edinburgh, Churchill Livingstone).
Wesley, S. V., Helliwell, C. A., Smith, N. A., Wang, M. B., Rouse, D. T., Liu, Q., Gooding, P. S., Singh, S. P., Abbott, D., Stoutjesdijk, P. A., et al. (2001). Construct design for efficient, effective and high-throughput gene silencing in plants. Plant J 27, 581-590.
Willis, W. D. (2001). Role of neurotransmitters in sensitization of pain responses. Ann N Y Acad Sci 933, 142-156.
Winston, W. M., Molodowitch, C., and Hunter, C. P. (2002). Systemic RNAi in C. elegans requires the putative transmembrane protein SID-1. Science 295, 2456-2459.
Wood, P. M. (1976). Separation of functional Schwann cells and neurons from normal peripheral nerve tissue. Brain Res 115, 361-375.
Yu, J. Y., DeRuiter, S. L., and Turner, D. L. (2002). RNA interference by expression of short-interfering RNAs and hairpin RNAs in mammalian cells. Proc Natl Acad Sci USA 99, 6047-6052.
Zamore, P. D., Tuschl, T., Sharp, P. A., and Bartel, D. P. (2000). RNAi: double-stranded RNA directs the ATP-dependent cleavage of mRNA at 21 to 23 nucleotide intervals. Cell 101, 25-33.
Zerangue, N., and Kavanaugh, M. P. (1996). Flux coupling in a neuronal glutamate transporter. Nature 383, 634-637.
Zheng, S., Ramachandran, B., Haigh, J. R., Palos, T. P., Steger, K., and Howard, B. D. (1996). The induction of ret by Wnt-1 in PC12 cells is atypically dependent on continual Wnt-1 expression. Oncogene 12, 555-562.
Zhuo, M. (2001). No pain, no gains. ScientificWorldJournal 1, 204-206.

What is claimed is:

1. A double-stranded RNA containing a sequence having the structure 5′-(N₁₇₋₂₅)-3′, which is at least 80% complementary to a fragment of the (m)RNA sequence of a member of the VGLUT family, wherein N is any base.

2. The RNA of claim 1, said RNA containing a sequence which is at least 90% complementary to a fragment of the (m)RNA sequence of a member of the VGLUT family.

3. The RNA of claim 1, said RNA containing a sequence which is at least 99% complementary to a fragment of the (m)RNA sequence of a member of the VGLUT family.

4. The RNA of claim 1, said RNA containing a sequence which is 100% complementary to a fragment of the (m)RNA sequence of a member of the VGLUT family.

5. A double-stranded RNA according to claim 1, comprising a sequence having the structure 5′-(N₁₉₋₂₅)-3′.

6. A double-stranded RNA according to 1, comprising a sequence having the structure 5′-(N₂₁₋₂₃)-3′.

7. A double-stranded RNA according to claim 1, wherein the sequence having the structure 5′-(N₂₁₋₂₃)-3′ is completely complementary to a fragment of the (m)RNA of a member of the VGLUT family, wherein N is any base.

8. A double-stranded RNA according to claim 1, wherein the double-stranded RNA (dsRNA) is complementary to nucleotide fragments on the (m)RNA of VGLUT1, VGLUT2 orVGLUT3.

9. A double-stranded RNA according to claim 1, comprising dsRNA against nucleotide fragments of the encoding region of VGLUT-(m)RNA, which are at least 50 nucleotides removed from the AUG initiating triplet of the encoding region of the (m)RNA.

10. The RNA of claim 9, wherein the dsRNA against nucleotide fragments of the encoding region of VGLUT-(m)RNA, are at least 70 nucleotides removed from the AUG initiating triplet of the encoding region of the (m)RNA.

11. The RNA of claim 9, wherein the dsRNA against nucleotide fragments of the encoding region of VGLUT-(m)RNA, are at least 100 nucleotides removed from the AUG initiating triplet of the encoding region of the (m)RNA.

12. A double-stranded RNA according to claim 8, wherein the dsRNA is complementary to nucleotide fragments of the encoding region of VGLUT-(m)RNA, which is at least 50 nucleotides removed from the 3′-terminus encoding region of the (m)RNA.

13. A double-stranded RNA (dsRNA) according to claim 1, wherein the double-stranded RNA is an siRNA, a long dsRNA comprising at least 30 nucleotides, a siRNA-based hairpin RNA, or an miRNA-based hairpin RNA.

14. A double-stranded RNA according to claim 1, wherein the dsRNA is complementary to fragments in the encoding region of the VGLUT-(m)RNA, containing the sequence AA.

15. A double-stranded RNA according to claim 1, wherein the dsRNA at the 5-terminus is complementary to the sequence AA.

16. A double-stranded RNA according to claim 1, wherein the dsRNA is complementary to nucleotide fragments in the non-encoding 5′ region of the VGLUT-(m)RNA.

17. A double-stranded RNA according to claim 1, wherein the dsRNA satisfies at least one of the following: (a) the GC content is at least 38%; (b) the dsRNA is not directed against regions which are at most 50 nucleotides removed from the initiating or terminating codon; (c) at most two successive guanidine radicals, and (d) the target sequence occurs only in the target gene in the genome to be investigated.

18. A double-stranded RNA according to claim 1, wherein the VGLUT target sequence comprises at least one sequence selected from the group consisting of:
(SEQ ID NO:16)
AATGCCTTTAGCTGGCATTCT,
(SEQ ID NO:17)
AATGGTCTGGTACATGTTTTG,
(SEQ ID NO:18)
AAAGTCCTGCAAAGCATCCTA,
(SEQ ID NO:20)
AAGAACGTAGGTACATAGAAG,
(SEQ ID NO:21)
AATTGTTGCAAACTTCTGCAG,
(SEQ ID NO:22)
AAATTAGCAAGGTTGGTATGC,
(SEQ ID NO:23)
AATTAGCAAGGTTGGTATGCT,
(SEQ ID NO:24)
AAGGTTGGTATGCTATCTGCT,
(SEQ ID NO:25)
AAGCAAGCAGATTCTTCAAC,
(SEQ ID NO:27)
AATGGGCATTTCGAATGGTGT,
(SEQ ID NO:28)
AATAAGTCACGTGAAGAGTGG,
(SEQ ID NO:31)
AATATTTGCCTCAGGAGAGAA,
(SEQ ID NO:32)
AAGTCTATGGTGCCACAACA,
(SEQ ID NO:34)
AAGACTCACATAGCTATAAGG,
(SEQ ID NO:19)
AAGTCCTGCAAAGCATCCTAC,
(SEQ ID NO:26)
AACCACTTGGATATCGCTCCA,
(SEQ ID NO:29)
AAGTCACGTGAAGAGTGGCAG,
(SEQ ID NO:30)
AAGAGTGGCAGTATGTCTTCC,
(SEQ ID NO:33)
AATGGAGGTTGGCCTAGTGGT,
(SEQ ID NO:35)
AATCTTGGAGTTGCCATTGTG,
(SEQ ID NO:38)
AATTCCAGGTGGTTTCATTTC,
(SEQ ID NO:39)
AACATCGACTCTGAACATGTT,
(SEQ ID NO:41)
AAGAGGTCTTTGGATTTGCAA,
(SEQ ID NO:42)
AATAAGTAAGGTGGGTCTCTT,
(SEQ ID NO:45)
AATCGTTGTACCTATTGGAGG,
(SEQ ID NO:47)
AAGAATGGCAGAATGTGTTCC,
(SEQ ID NO:48)
AATCATTGACCAGGACGAATT,
(SEQ ID NO:49)
AACTCAACCATGAGAGTTTTG,
(SEQ ID NO:50)
AAAGAAGATGTCTTATGGAGC,
(SEQ ID NO:52)
AAGAGCTGACATCCTACCAGA,
(SEQ ID NO:36)
AACCGGAAATTCAGACAGCAC,
(SEQ ID NO:37)
AAACAGTGGGCCTTATCCATG,
(SEQ ID NO:40)
AAGGTTTAGTGGAGGGTGTGA,
(SEQ ID NO:43)
AAGTAAGGTGGGTCTCTTGTC,
(SEQ ID NO:44)
AAGGTGGGTCTCTTGTCAGCA,
(SEQ ID NO:46)
AAGACCCGTGAAGAATGGCAG and
(SEQ ID NO:14)
AACGTGCGCAAGTTGATGAAC.

19. A double-stranded RNA according to claim 1, wherein the dsRNA is chemically modified.

20. A double-stranded RNA according to claim 1, wherein the double-stranded RNA suppresses the expression of at least one member of the VGLUT family in the cell by at least 50% (dsRNA).

21. A double-stranded RNA according to claim 1, wherein the dsRNA is complementary with nucleotide fragments of the (m)RNA of a member of the VGLUT family of mammals.

22. The double-stranded RNA according to claim 21, wherein the dsRNA is complementary with nucleotide fragments of the (m)RNA of a member of the VGLUT family in humans.

23. A double-stranded RNA according to claim 1, wherein the dsRNA comprises at least one blunt end.

24. A double-stranded RNA according to claim 1, wherein the dsRNA comprises at least one overhanging end.

25. A double-stranded RNA according to claim 24, wherein the overhanging end has the nucleotides dTdT.

26. A double-stranded RNA according to claim 24, wherein the overhanging end comprises at least two overhanging nucleotides.

27. A double-stranded RNA according to claim 24, wherein the overhanging end comprises from 2 to 10 overhanging nucleotides.

28. A double-stranded RNA according to claim 24, wherein the overhanging end comprises from 2 to 5 overhanging nucleotides.

29. A double-stranded RNA according to claim 24, wherein the overhanging nucleotides are attached to the double strand complementary with the (m)RNA sequence of a member of the VGLUT family.

30. A double-stranded RNA according to claim 29, wherein the overhanging nucleotides are deoxidized thymidines or uracils.

31. A double-stranded RNA according to claim 1, wherein the sequence complementary with the dsRNA sequence is 5′-AAGUGUACUUUAGGCAAAGGG-3′ (SEQ ID NO: 110).

32. A double-stranded RNA according to claim 31, of which the sense strand comprises the sequence 5′-GUGUACUUUAGGCAAAGGGdTdT-3′ (SEQ ID NO: 111) and of which the antisense strand comprises the sequence 5′-CCCUUUGCCUAAAGUACACdTdT-3′ (SEQ ID NO: 112).

33. A cell containing at least one dsRNA according to claim 1.

34. A pharmaceutical composition comprising at least one dsRNA according to claim 1 or a cell containing at least one dsRNA according to claim 1, and at least one pharmaceutically acceptable auxiliary or additive.

35. A diagnostic reagent comprising at least one dsRNA according to claim 1 or a cell containing at least one dsRNA according to claim 1 and, optionally, at least one suitable additive.

36. A method of alleviating pain in a mammal said method comprising the step of administering to said mammal a pain-alleviating amount of a dsRNA according to claim 1 or of a cell containing at least one dsRNA according to claim 1.

37. The method of claim 36, wherein said pain is chronic pain, tactile allodynia, thermally triggered pain or inflammatory pain.

38. A method of treating urinary incontinence, neurogenic bladder symptoms, pruritus, tumors, inflammation or any disease symptoms associated with the physiological function of VGLUT family members in a mammal said method comprising the step of administering to said mammal a dsRNA according to claim 1 or of a cell containing at least one dsRNA according to claim 1.

39. The method of claim 38, wherein said method is a method for treating inflammation or symptoms associated with the physiological function of VGLUT family members.

40. The method of claim 38, wherein said method is a method for treating asthma.

41. The method of claim 38, wherein said dsRNA is administered through in vivo or in vitro gene therapy.

42. A process for inhibiting the expression of at least one VGLUT family member in a cell, comprising introducing a dsRNA according to claim 1 into the cell, wherein a strand of the dsRNA comprises a region complementary with the (m)RNA of a member of the VGLUT family, and wherein the complementary region comprises less than 25 successive nucleotide pairs.

43. A process according to claim 42, wherein the dsRNA is enclosed in micellar structures.

44. The process of claim 43, wherein said micellar structures are liposomes.

45. A process according to claim 42, wherein the dsRNA is enclosed in viral natural capsids or in chemically or enzymatically produced capsids or structures derived therefrom.

46. A process for identifying dsRNA with a pain-modulating function, comprising measuring, in a binding assay, the binding constant between a dsRNA according to claim 1, as a test substance, and an (m)RNA of a member of the VGLUT family.

47. The process of claim 46, wherein the dsRNA according to claim 1 is marked.

48. A process for identifying a pain-modulating substance, said process comprising:
(a) over-expressing a VGLUT, in a test cell;
(b) manipulating at least one cell as a test cell with at least one dsRNA according to claim 1;
(b′) simultaneously manipulating at least one identical cell as a control cell, the control cell either being manipulated by addition of dsRNA or being manipulated with an altered dsRNA not corresponding to claim 1, or process step (b′) being omitted,
(c) simultaneously incubating the at least one manipulated test cell according to process step (b) and optionally the at least one control cell according to process step (b′) under suitable conditions,
(d) measuring the concentration of expressed VGLUT in the at least one test cell and optionally the at least one control cell, the binding of the test substance on the VGLUT-(m)RNA in the at least one test cell, of at least one of the functional parameters of the test cell altered by the effect of the test substance or of at least one signal correlating with the expression level of VGLUT in the at least one test cell, and
(e) identifying potentially pain-modulating substances via the extent of the difference between the measured value in the at least one test cell and the measured value in the at least one control cell.

49. The process of claim 48, wherein said VGLUT is VGLUT1, VGLUT2 orVGLUT3.

50. A process according to claim 48, wherein the cell used in process step (a) is genetically manipulated.

51. A process according to claim 48, wherein genetic manipulation allows the measurement of at least one functional parameter altered by the test substance and said process includes the step of measuring at least one functional parameter altered by the test substance.

52. A process according to claim 48, wherein a form of a member of the VGLUT family is expressed or a reporter gene is introduced by genetic manipulation.

53. The process of claim 52, wherein the member of the VGLUT family expressed is VGLUT1, VGLUT2 or VGLUT3 not endogenously expressed in the cell.

54. A process according to claim 48, wherein ≧8 hours elapse between the simultaneous process steps (b) and (b′) and the process step (c).

55. A process according to claim 48, wherein ≧12 hours elapse between the simultaneous process steps (b) and (b′) and the process step (c).

56. A process according to claim 48, wherein ≧24 hours elapse between the simultaneous process steps (b) and (b′) and the process step (c).


SEQUENCE LISTING (112 sequences)

SEQ ID NO: 1 - RNA, 17 nt, Artificial sequence
  Chemcially synthesized
  misc_feature (1)..(17): n = a, u, g, c, unknown or other
nnnnnnnnnnnnnnnnn

SEQ ID NO: 2 - RNA, 18 nt, Artificial sequence
  Chemcially synthesized
  misc_feature (1)..(18): n = a, u, g, c, unknown or other
nnnnnnnnnnnnnnnnnn

SEQ ID NO: 3 - RNA, 19 nt, Artificial sequence
  Chemcially synthesized
  misc_feature (1)..(19): n = a, u, g, c, unknown or other
nnnnnnnnnnnnnnnnnnn

SEQ ID NO: 4 - RNA, 20 nt, Artificial sequence
  Chemcially synthesized
  misc_feature (1)..(20): n = a, u, g, c, unknown or other
nnnnnnnnnnnnnnnnnnnn

SEQ ID NO: 5 - RNA, 21 nt, Artificial sequence
  Chemcially synthesized
  misc_feature (1)..(21): n = a, u, g, c, unknown or other
nnnnnnnnnnnnnnnnnnnnn

SEQ ID NO: 6 - RNA, 22 nt, Artificial sequence
  Chemcially synthesized
  misc_feature (1)..(22): n = a, u, g, c, unknown or other
nnnnnnnnnnnnnnnnnnnnnn

SEQ ID NO: 7 - RNA, 23 nt, Artificial sequence
  Chemcially synthesized
  misc_feature (1)..(23): n = a, u, g, c, unknown or other
nnnnnnnnnnnnnnnnnnnnnnn

SEQ ID NO: 8 - RNA, 24 nt, Artificial sequence
  Chemcially synthesized
  misc_feature (1)..(24): n = a, u, g, c, unknown or other
nnnnnnnnnnnnnnnnnnnnnnnn

SEQ ID NO: 9 - RNA, 25 nt, Artificial sequence
  Chemcially synthesized
  misc_feature (1)..(25): n = a, u, g, c, unknown or other
nnnnnnnnnnnnnnnnnnnnnnnnn

SEQ ID NO: 10 - DNA, 23 bp, Artificial sequence
  Sequence motif of a preferred VGLUT-dsRNA according to the invention (p. 9 of the specification). The sequence motif is AAN(19)TT
  misc_feature (3)..(21): n = a, u, g, c, unknown or other
aannnnnnnnnnnnnnnnnnntt

SEQ ID NO: 11 - DNA, 23 bp, Artificial sequence
  Sequence motif of a preferred VGLUT-dsRNA according to the invention (p. 9 of the specification). The sequence motif is NAN(19)NN.
  misc_feature (1)..(23): n = a, t/u, g, c, unknown or other
nannnnnnnnnnnnnnnnnnnnn

SEQ ID NO: 12 - DNA, 23 bp, Artificial sequence
  Sequence motif of a preferred VGLUT-dsRNA according to the invention (p. 9 of the specification). The sequence motif is NARN(17)YNN.
  misc_feature (1)..(23): n = a, t/u, g, c, unknown or other; y = c or t; r = a or g
narnnnnnnnnnnnnnnnnnynn

SEQ ID NO: 13 - DNA, 23 bp, Artificial sequence
  Sequence motif of a preferred VGLUT-dsRNA according to the invention (p. 9 of the specification). The sequence motif is NANN(17)YNN.
  misc_feature (1)..(23): n = a, t/u, g, c, unknown or other; y = c or t.
nannnnnnnnnnnnnnnnnnynn

SEQ ID NO: 14 - DNA, 21 bp, Homo sapiens
  misc_feature (1)..(21): DNA target sequence of VGLUT1 (Fig. 26).
aacgtgcgcaagttgatgaac

SEQ ID NO: 15 - DNA, 21 bp, Homo sapiens
  misc_feature (1)..(21): DNA target sequence of VGLUT1 (Fig. 26).
aagttgatgaactgcggaggc

SEQ ID NO: 16 - DNA, 21 bp, Homo sapiens
  misc_feature (1)..(21): DNA target sequence of VGLUT2 (Fig. 26).
aatgcctttagctggcattct

SEQ ID NO: 17 - DNA, 21 bp, Homo sapiens
  misc_feature (1)..(21): One of the DNA target sequences of VGLUT2 (Fig. 26).
aatggtctggtacatgttttg

SEQ ID NO: 18 - DNA, 21 bp, Homo sapiens
  misc_feature (1)..(21): One of the DNA target sequences of VGLUT2 (Fig. 26).
aaagtcctgcaaagcatccta

SEQ ID NO: 19 - DNA, 21 bp, Homo sapiens
  misc_feature (1)..(21): One of the DNA target sequences of VGLUT2 (Fig. 26).
aagtcctgcaaagcatcctac

SEQ ID NO: 20 - DNA, 21 bp, Homo sapiens
  misc_feature (1)..(21): One of the DNA target sequences of VGLUT2 (Fig. 26).
aagaacgtaggtacatagaag

SEQ ID NO: 21 - DNA, 21 bp, Homo sapiens
  misc_feature (1)..(21): One of the DNA target sequences of VGLUT2 (Fig. 26).
aattgttgcaaacttctgcag

SEQ ID NO: 22 - DNA, 21 bp, Homo sapiens
  misc_feature (1)..(21): One of the DNA target sequences of VGLUT2 (Fig. 26).
aaattagcaaggttggtatgc

SEQ ID NO: 23 - DNA, 21 bp, Homo sapiens
  misc_feature (1)..(21): One of the DNA target sequences of VGLUT2 (Fig. 26).
aattagcaaggttggtatgct

SEQ ID NO: 24 - DNA, 21 bp, Homo sapiens
  misc_feature (1)..(21): One of the DNA target sequences of VGLUT2 (Fig. 26).
aaggttggtatgctatctgct

SEQ ID NO: 25 - DNA, 21 bp, Homo sapiens
  misc_feature (1)..(21): One of the DNA target sequences of VGLUT2 (Fig. 26).
aagcaagcagattctttcaac

SEQ ID NO: 26 - DNA, 21 bp, Homo sapiens
  misc_feature (1)..(21): One of the DNA target sequences of VGLUT2 (Fig. 26).
aaccacttggatatcgctcca

SEQ ID NO: 27 - DNA, 21 bp, Homo sapiens
  misc_feature (1)..(21): One of the DNA target sequences of VGLUT2 (Fig. 26).
aatgggcatttcgaatggtgt

SEQ ID NO: 28 - DNA, 21 bp, Homo sapiens
  misc_feature (1)..(21): One of the DNA target sequences of VGLUT2 (Fig. 26).
aataagtcacgtgaagagtgg

SEQ ID NO: 29 - DNA, 21 bp, Homo sapiens
  misc_feature (1)..(21): One of the DNA target sequences of VGLUT2 (Fig. 26).
aagtcacgtgaagagtggcag

SEQ ID NO: 30 - DNA, 21 bp, Homo sapiens
  misc_feature (1)..(21): One of the DNA target sequences of VGLUT2 (Fig. 26).
aagagtggcagtatgtcttcc

SEQ ID NO: 31 - DNA, 21 bp, Homo sapiens
  misc_feature (1)..(21): One of the DNA target sequences of VGLUT2 (Fig. 26).
aatatttgcctcaggagagaa

SEQ ID NO: 32 - DNA, 21 bp, Homo sapiens
  misc_feature (1)..(21): One of the DNA target sequences of VGLUT2 (Fig. 26).
aagtcttatggtgccacaaca

SEQ ID NO: 33 - DNA, 21 bp, Homo sapiens
  misc_feature (1)..(21): One of the DNA target sequences of VGLUT2 (Fig. 26).
aatggaggttggcctagtggt

SEQ ID NO: 34 - DNA, 21 bp, Homo sapiens
  misc_feature (1)..(21): One of the DNA target sequences of VGLUT2 (Fig. 26).
aagactcacatagctataagg

SEQ ID NO: 35 - DNA, 21 bp, Homo sapiens
  misc_feature (1)..(21): One of the DNA target sequences of VGLUT3 (Fig. 26).
aatcttggagttgccattgtg

SEQ ID NO: 36 - DNA, 21 bp, Homo sapiens
  misc_feature (1)..(21): One of the DNA target sequences of VGLUT3 (Fig. 26).
aaccggaaattcagacagcac

SEQ ID NO: 37 - DNA, 21 bp, Homo sapiens
  misc_feature (1)..(21): One of the DNA target sequences of VGLUT3 (Fig. 26).
aaacagtgggccttatccatg

SEQ ID NO: 38 - DNA, 21 bp, Homo sapiens
  misc_feature (1)..(21): One of the DNA target sequences of VGLUT3 (Fig. 26).
aattccaggtggtttcatttc

SEQ ID NO: 39 - DNA, 21 bp, Homo sapiens
  misc_feature (1)..(21): One of the DNA target sequences of VGLUT3 (Fig. 26).
aacatcgactctgaacatgtt

SEQ ID NO: 40 - DNA, 21 bp, Homo sapiens
  misc_feature (1)..(21): One of the DNA target sequences of VGLUT3 (Fig. 26).
aaggtttagtggagggtgtga

SEQ ID NO: 41 - DNA, 21 bp, Homo sapiens
  misc_feature (1)..(21): One of the DNA target sequences of VGLUT3 (Fig. 26).
aagaggtctttggatttgcaa

SEQ ID NO: 42 - DNA, 21 bp, Homo sapiens
  misc_feature (1)..(21): One of the DNA target sequences of VGLUT3 (Fig. 26).
aataagtaaggtgggtctctt

SEQ ID NO: 43 - DNA, 21 bp, Homo sapiens
  misc_feature (1)..(21): One of the DNA target sequences of VGLUT3 (Fig. 26).
aagtaaggtgggtctcttgtc

SEQ ID NO: 44 - DNA, 21 bp, Homo sapiens
  misc_feature (1)..(21): One of the DNA target sequences of VGLUT3 (Fig. 26).
aaggtgggtctcttgtcagca

SEQ ID NO: 45 - DNA, 21 bp, Homo sapiens
  misc_feature (1)..(21): One of the DNA target sequences of VGLUT3 (Fig. 26).
aatcgttgtacctattggagg

SEQ ID NO: 46 - DNA, 21 bp, Homo sapiens
  misc_feature (1)..(21): One of the DNA target sequences of VGLUT3 (Fig. 26).
aagacccgtgaagaatggcag

SEQ ID NO: 47 - DNA, 21 bp, Homo sapiens
  misc_feature (1)..(21): One of the DNA target sequences of VGLUT3 (Fig. 26).
aagaatggcagaatgtgttcc

SEQ ID NO: 48 - DNA, 21 bp, Homo sapiens
  misc_feature (1)..(21): One of the DNA target sequences of VGLUT3 (Fig. 26).
aatcattgaccaggacgaatt

SEQ ID NO: 49 - DNA, 21 bp, Homo sapiens
  misc_feature (1)..(21): One of the DNA target sequences of VGLUT3 (Fig. 26).
aactcaaccatgagagttttg

SEQ ID NO: 50 - DNA, 21 bp, Homo sapiens
  misc_feature (1)..(21): One of the DNA target sequences of VGLUT3 (Fig. 26).
aaagaagatgtcttatggagc

SEQ ID NO: 51 - DNA, 21 bp, Homo sapiens
  misc_feature (1)..(21): One of the DNA target sequences of VGLUT3 (Fig. 26).
aagagctgacatcctaccaga

SEQ ID NO: 52 - DNA, 17 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(17): n = a, t/u, g, c, unknown or other.
aannnnnnnnnnnnnnn

SEQ ID NO: 53 - DNA, 18 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(18): n = a, t/u, g, c, unknown or other.
aannnnnnnnnnnnnnnn

SEQ ID NO: 54 - DNA, 19 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(19): n = a, t/u, g, c, unknown or other.
aannnnnnnnnnnnnnnnn

SEQ ID NO: 55 - DNA, 20 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(20): n = a, t/u, g, c, unknown or other
aannnnnnnnnnnnnnnnnn

SEQ ID NO: 56 - DNA, 21 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(21): n = a, t/u, g, c, unknown or other
aannnnnnnnnnnnnnnnnnn

SEQ ID NO: 57 - DNA, 22 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(22): n = a, t/u, g, c, unknown or other
aannnnnnnnnnnnnnnnnnnn

SEQ ID NO: 58 - DNA, 23 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(23): n = a, t/u, g, c, unknown or other
aannnnnnnnnnnnnnnnnnnnn

SEQ ID NO: 59 - DNA, 24 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(24): n = a, t/u, g, c, unknown or other
aannnnnnnnnnnnnnnnnnnnnn

SEQ ID NO: 60 - DNA, 25 bp, Artificial sequence
  Sequence of a dsRNA, wherein the sequence of the dsRNA comprises the nucleotides AA at its 5′-end (p.12 of the specification). Sequence motif 5′AAN(15-23)
  misc_feature (3)..(25): n = a, t/u, g, c, unknown or other
aannnnnnnnnnnnnnnnnnnnnnn

SEQ ID NO: 61 - DNA, 17 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(15): n = a, t/u, g, c, unknown or other
nnnnnnnnnnnnnnntt

SEQ ID NO: 62 - DNA, 18 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(16): n = a, t/u, g, c, unknown or other
nnnnnnnnnnnnnnnntt

SEQ ID NO: 63 - DNA, 19 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(17): n = a, t/u, g, c, unknown or other
nnnnnnnnnnnnnnnnntt

SEQ ID NO: 64 - DNA, 20 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(18): n = a, t/u, g, c, unknown or other
nnnnnnnnnnnnnnnnnntt

SEQ ID NO: 65 - DNA, 21 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(19): n = a, t/u, g, c, unknown or other
nnnnnnnnnnnnnnnnnnntt

SEQ ID NO: 66 - DNA, 22 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(20): n = a, t/u, g, c, unknown or other
nnnnnnnnnnnnnnnnnnnntt

SEQ ID NO: 67 - DNA, 23 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(21): n = a, t/u, g, c, unknown or other
nnnnnnnnnnnnnnnnnnnnntt

SEQ ID NO: 68 - DNA, 24 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(22): n = a, t/u, g, c, unknown or other
nnnnnnnnnnnnnnnnnnnnnntt

SEQ ID NO: 69 - DNA, 25 bp, Artificial sequence
  Sequence of a dsRNA, wherein the sequence of the dsRNA comprises the nucleotides TT at its 3′-end (p.12 of the specification). Sequence motif 3′TTN(15-23)
  misc_feature (1)..(23): n = a, t/u, g, c, unknown or other
nnnnnnnnnnnnnnnnnnnnnnntt

SEQ ID NO: 70 - RNA, 21 nt, Rattus norvegicus
  misc_feature (1)..(21): Target sequence of VGLUT2-mRNA (p. 17 of the specification).
aaggcuccgcuaugcgacugu

SEQ ID NO: 71 - DNA, 21 bp, Artificial sequence
  Sense strand of dsRNA, directed to the afore disclosed target sequence of VGLUT2-mRNA from rat (having an overhanging TT at its 3′-terminus). (p. 17 of the specification).
ggcuccgcuaugcgacugutt

SEQ ID NO: 72 - DNA, 21 bp, Artificial sequence
  Antisense strand of dsRNA, directed to the afore disclosed target sequence of VGLUT2-mRNA from rat (having an overhanging TT at its 3′-terminus). (p. 17 of the specification).
acagucgcauagcggagcctt

SEQ ID NO: 73 - DNA, 21 bp, Artificial sequence
  si-rVGLUT1 739-759 EGT (p. 36 of the specification).
agcgccaagcucaugaacctt

SEQ ID NO: 74 - DNA, 21 bp, Artificial sequence
  si-rVGLUT1 739-759 EGT (p. 36 of the specification)
gguucaugagcuuggcgcutt

SEQ ID NO: 75 - DNA, 21 bp, Artificial sequence
  si-rVGLUT2 100-120 EGT (active siRNA) (p. 36 of the specification)
gcaggauaaccgagagacctt

SEQ ID NO: 76 - DNA, 21 bp, Artificial sequence
  si-rVGLUT2 100-120 EGT (active siRNA) (p. 36 of the specification)
ggucucucgguuauccugctt

SEQ ID NO: 77 - DNA, 21 bp, Artificial sequence
  si-rVGLUT3 220-240 EGT (p. 36 of the specification)
gcgguacaucaucgcuguctt

SEQ ID NO: 78 - DNA, 21 bp, Artificial sequence
  si-rVGLUT3 220-240 EGT (p. 36 of the specification)
gacagcgaugauguaccgctt

SEQ ID NO: 79 - DNA, 21 bp, Artificial sequence
  si-VGLUT2 MM EGT (siRNA control ) (p. 36 of the specification)
ggacuagcaaagcgagccatt

SEQ ID NO: 80 - DNA, 21 bp, Artificial sequence
  si-VGLUT2 MM EGT (siRNA control ) (p. 36 of the specification)
uggcucgcuuugcuagucctt

SEQ ID NO: 81 - DNA, 21 bp, Artificial sequence
  si-rVGLUT2 100-120 AMB (active siRNA) (p. 36 of the specification)
gcaggauaaccgagagacctt

SEQ ID NO: 82 - DNA, 21 bp, Artificial sequence
  si-rVGLUT2 100-120 AMB (active siRNA) (p. 36 of the specification)
ggucucucgguuauccugctt

SEQ ID NO: 83 - DNA, 21 bp, Artificial sequence
  si-rVGLUT2 166-186 AMB (p. 36 of the specification)
ggcuccgcuaugcgacugutt

SEQ ID NO: 84 - DNA, 21 bp, Artificial sequence
  si-rVGLUT2 166-186 AMB (p. 37 of the specification)
acagucgcauagcggagcctt

SEQ ID NO: 85 - DNA, 21 bp, Rattus norvegicus
  misc_feature (1)..(21): Target sequence of VGLUT2 from rat (p. 18 of the specification).
aagcaggataaccgagagacc

SEQ ID NO: 86 - RNA, 21 nt, Rattus norvegicus
  misc_feature (1)..(21): Target sequence of VGLUT2-mRNA from rat (p. 48 of the specification).
aagcaggauaaccgagagacc

SEQ ID NO: 87 - DNA, 21 bp, Artificial sequence
  Sense strand of siRNA, directed to the afore disclosed target sequence of VGLUT2 or to the target sequence of VGLUT2-mRNA, respectively (p. 18 u. 48 of the specification).
  misc_feature (20)..(20): T = dT (desoxy Thymidin)
  misc_feature (21)..(21): T = dT (desoxy Thymidin)
gcaggauaaccgagagacctt

SEQ ID NO: 88 - DNA, 21 bp, Artificial sequence
  Antisense strand of siRNA, directed to the afore disclosed target sequence of VGLUT2 or to the target sequence of VGLUT2-mRNA, respectively (p. 18 u. 48 of the specification).
  misc_feature (20)..(20): T = dT (desoxy Thymidin)
  misc_feature (21)..(21): T = dT (desoxy Thymidin)
ggucucucgguuauccugctt

SEQ ID NO: 89 - DNA, 21 bp, Artificial sequence
  Target sequence (no VGLUT2 sequence) of siRNA control in Example 4. (p. 48 of the specification).
aaggactagcaaagcgagcca

SEQ ID NO: 90 - DNA, 21 bp, Artificial sequence
  Sense strand of double stranded siRNA control, directed to the afore disclosed target sequence. (p. 48 of the specification).
  misc_feature (20)..(20): T = dT (desoxy Thymidin)
  misc_feature (21)..(21): T = dT (desoxy Thymidin)
ggacuagcaaagcgagccatt

SEQ ID NO: 91 - DNA, 21 bp, Artificial sequence
  Antisense strand of double stranded siRNA control, directed to the afore disclosed target sequence. (p. 48 of the specification).
  misc_feature (20)..(20): T = dT (desoxy Thymidin)
  misc_feature (21)..(21): T = dT (desoxy Thymidin)
uggcucgcuuugcuagucctt

SEQ ID NO: 92 - DNA, 19 bp, Artificial sequence
  PCR primer rVGluT1(2_4)F; (p. 56 of the specification).
tctgggtttctgcatcagc

SEQ ID NO: 93 - DNA, 20 bp, Artificial sequence
  PCR primer rVGluT1(2_4)R; (p. 56 of the specification).
ccatgtatgaggccgacagt

SEQ ID NO: 94 - DNA, 20 bp, Artificial sequence
  PCR primer rVGluT2(8_9)F; (p. 56 of the specification).
aagaccccatggaggaagtt

SEQ ID NO: 95 - DNA, 20 bp, Artificial sequence
  PCR primer rVGluT2(8_9)R; (p. 56 of the specification).
attgtcatgaccaggtgtgg

SEQ ID NO: 96 - DNA, 20 bp, Artificial sequence
  PCR primer rVGluT3(4_5)F; (p. 56 of the specification).
atccagagacggtgggtctt

SEQ ID NO: 97 - DNA, 20 bp, Artificial sequence
  PCR primer rVGluT3(4_5)R; (p. 56 of the specification).
atgacacagccgtaatgcac

SEQ ID NO: 98 - DNA, 20 bp, Artificial sequence
  PCR primer rGAPDH(7_8)F; (p. 56 of the specification).
atcctgggctacactgagga

SEQ ID NO: 99 - DNA, 20 bp, Artificial sequence
  PCR primer rGAPDH(7_8)R; (p. 56 of the specification).
atgtaggccatgaggtccac

SEQ ID NO: 100 - DNA, 29 bp, Artificial sequence
  Primer rVGluT2 TS10 as; (p. 56 of the specification).
aagcaggataaccgagagacccctgtctc

SEQ ID NO: 101 - DNA, 29 bp, Artificial sequence
  Primer rVGluT2 TS10 s; (p. 56 of the specification).
aaggtctctcggttatcctgccctgtctc

SEQ ID NO: 102 - DNA, 29 bp, Artificial sequence
  Primer rVGluT2 TS14 as; (p. 57 of the specification).
aaggctccgctatgcgactgtcctgtctc

SEQ ID NO: 103 - DNA, 29 bp, Artificial sequence
  Primer rVGluT2 TS14 s; (p. 57 of the specification).
aaacagtcgcatagcggagcccctgtctc

SEQ ID NO: 104 - DNA, 1683 bp, Unknown
  Coding sequence of VGLUT1, having the accession number U07609 (Fig. 5).
atggagttccggcaggaggagtttcggaagctggcggggcgcgccctggggaggctgcaccggttactggagaagcggcaggaaggcgcggagacattggagctgagcgccgacgggcgcccagtgaccacacacacgcgggacccgccggtggtggactgcacttgctttggcctccctcgccgctacatcatcgcgatcatgagcggtctgggtttctgcatcagctttggcatccgctgcaacctgggcgtggccatcgtatccatggtcaacaacagtacaacccaccgtgggggccacgtggtggtgcagaaagcccagttcaactgggatccagagactgtcggcctcatacatggctcctttttctgggggtacattgtcactcagattcctggaggatttatctgccaaaaattcgcagccaacagggtctttggctttgccattgtggctacctccaccctaaatatgttgatcccttcagcagcccgtgttcactatggctgtgtcatcttcgtgaggatccttcagggattggtggagggggtcacataccctgcttgccatggcatctggagcaaatgggcccctcccttagaacggagtcggctggcgacgacagccttttgcggttcctatgccggggcagtggttgccatgcctctggctggggtcctggtacagtattcaggatggagttctgtcttctatgtctatggcagcttcgggatcttttggtacctgttctggttgcttgtctcctacgagtcacctgcactacaccccagcatctccgaggaggagcgcaaatacattgaggatgccatcggagaaagcgccaagctcatgaaccctgttacgaagtttaacacaccctggaggcgcttctttacctccatgccggtctatgccatcattgtcgccaacttttgccgcagctggactttctacctgctcctcatctcccagcccgcctactttgaagaagtgttcggctttgagatcagcaaggtgggactggtgtcggcactgcctcaccttgtcatgactatcatcgtacccatcggaggccagatcgccgacttcctgcgcagtcgtcatataatgtccacgaccaatgtgcgaaagctgatgaactgcgggggtttcgggatggaagctacgctgctgctggtggtcggatactcacactccaagggcgtggccatctccttcctggtcctggctgtgggcttcagtggctttgctatctctgggtttaacgtgaaccacttggacatcgcccctcgatatgccagcatcttgatgggcatttccaatggcgtgggcacactgtctgggatggtgtgccccatcatcgtgggtgcaatgaccaagcacaagacgcgggaggagtggcagtacgtgttcctcatagcctccctggtgcactatggaggtgtcatcttctatggggtctttgcttcgggagagaaacagccgtgggcagagccggaggagatgagcgaggagaagtgtggctttgttggccacgaccagctggctggcagtgacgaaagtgaaatggaagacgaggttgagcccccgggggcaccccccgcacctccgccttcctacggggccacacacagcacagttcagcctccaaggcccccaccccctgtccgggactactga

SEQ ID NO: 105 - DNA, 1749 bp, Rattus norvegicus
  misc_feature (1)..(1749): Coding sequence of VGLUT2,; Genbank accession number NM_053427 (Figur 6).
atggagtcggtaaaacaaaggattttggccccggggaaagaggggataaagaattttgctggaaaatccctcggacagatctacagggtgctggagaagaagcaggataaccgagagaccatcgagctgacagaggacggcaagcccctggaggtgcctgagaagaaggctccgctatgcgactgtacgtgcttcggcctgccgcgccgctacatcatagccatcatgagcggcctcggcttctgcatctcctttggtatccgctgtaacctgggtgtggccattgtggacatggtcaacaacagcaccatccaccggggtggcaaagttatcaaggagaaagccaagtttaactgggaccccgagactgtggggatgattcacgggtcgttcttctggggctatatcatcacgcagattccgggcggatacatcgcatcgcgactggctgctaaccgggtctttggggctgccatactgcttacctctaccctcaatatgctgatcccatctgcagccagagtgcattatggatgcgtcatctttgttagaatattgcaaggacttgtggagggcgtcacctacccagcctgtcacgggatatggagcaagtgggcccctcctttggagaggagtaggttggctaccacctccttctgtggttcctatgctggagcagtcattgcaatgcccctagctggtatcctggtgcagtacactggatggtcttcagtattttacgtatatggaagctttggtatggtctggtatatgttctggcttctggtgtcttacgagagccccgcaaagcatccaaccataacagacgaagaacgtaggtacatagaagagagcatcggggagagcgcaaatctgttaggagcaatggagaaattcaagaccccatggaggaagtttttcacatccatgcccgtctatgcgataattgttgcaaacttctgcaggagttggactttttatttactgctcatcagtcaaccagcttatttcgaggaggtttttggatttgaaatcagcaaggttggcatgttgtctgcggtcccacacctggtcatgacaatcattgtgcctatcggggggcaaattgcagactttctaaggagcaagcaaattctttcaacaactacagtgcgaaagatcatgaactgcgggggttttggcatggaagccacactgcttctggttgttggctactctcatactagaggggtggccatctccttcttggtgcttgcagtgggattcagtggatttgctatctctggtttcaatgtgaaccacttggatattgccccgagatatgccagtatcttaatgggcatttcaaatggtgttggcacgctgtcgggaatggtctgcccgatcattgttggtgcaatgacgaagaacaagtcccgtgaagaatggcagtatgtcttcctcatcgctgcactggtccactatggtggagtcatattttatgcactatttgcctcaggagagaagcaaccttgggcagaccctgaggaaacaagcgaagaaaagtgtggcttcattcatgaagatgaactggatgaagaaacgggggacatcactcagaattacataaattacggtaccaccaaatcctacggcgccacctcacaggagaacggaggctggcctaacggctgggagaaaaaggaagaatttgtgcaagaaagtgcgcaagacgcgtactcctataaggaccgagatgattattcataa

SEQ ID NO: 106 - DNA, 1767 bp, Unknown
  Coding sequence of VGLUT3 having the accession number AJ491795 (Fig. 7).
atgccatttaacgcatttgataccttcaaagaaaaaattttgaaacccgggaaggaaggagtgaagaatgccgtgggagattcgctggggatcttacaaagaaaactcgatgggaccaacgaggagggagatgccattgagctgagtgaggaaggaaggcccgtgcagacatccagagcccgagcccctgtgtgcgactgcagctgctgtggcatccccaagcggtacatcatcgctgtcatgagtggcctgggattctgcatttcctttgggattcggtgcaaccttggagtggccattgtggaaatggtcaacaatagcactgtgtatgtggatgggaaaccggaaatccagacagcacagtttaactgggatccagagacggtgggtcttatccatggatcttttttctggggttatattgtgacacaaattcccggtggcttcatttcaaacaagtttgctgctaacagggtctttggagctgccatcttcttgacgtcaaccctgaacatgttcatcccttccgcggccagggtgcattacggctgtgtcatgtgtgtgaggattttgcagggtctggtggagggtgtgacctacccagcctgccacgggatgtggagtaagtgggcacctcccctggagagaagtcgtctagccacaacctctttttgtggttcctatgccggggcagtcgttgctatgccccttgcaggagtattggtgcagtacattggctgggcctctgtcttttatatttacgggatgtttggaattatttggtacatgttttggctgctgcaggcttatgagtgtccagcagttcacccaacaatatccaatgaagaacggacctacatagagacaagtataggagaaggcgccaacttggccagtctgagcaaattcaacacaccatggagaaggtttttcacatccttgcctgtctatgccattattgtggcaaacttttgtagaagctggaccttctatttgctcttaataagtcagcctgcttactttgaagaggtctttgggtttgcaataagtaaggtgggtctcttgtcagctgtcccacacatggtgatgacaatcgtggtacccattggaggacaactggctgattatttaagaagccgaaagattttgaccacaactgctgtcagaaagatcatgaattgtggaggctttggcatggaggcaaccttgctcctggtggttgggttttcccataccaaaggagtggctatctccttcctggtgcttgctgtaggatttagtggctttgcaatttcaggtttcaatgtcaaccacctggacattgctccacgatatgccagcatcctcatggggatctcaaatggcgtgggaaccctctctggaatggtttgtcccctcattgttggtgcaatgacaaagcacaagacccgggaagaatggcagaatgtgttcctcatagcagccctggtgcactacagtggagtcatcttctacggggtctttgcttctggggaaaaacaggactgggctgatccagagaatctctctgaggagaaatgtggaatcattgaccaagatgaattagccgaggaaacagaactcaaccacgaggctttcgtaagtcccagaaagaagatgtcttatggagccaccacccagaattgtgaggtccagaagacggatcggagacaacagagagaatccgccttcgagggggaggagccattatcctaccagaatgaagaggacttttcagaaacatcttaa

SEQ ID NO: 107 - DNA, 2959 bp, Homo sapiens
  misc_feature (1)..(2959): VGLUT1 (Fig. 27A)
acttgcagcctccttccccccgagcggagctgcggggccggccgggccggggcggaccccgggaacccggacgcggccgcccgggcccgcgggcggggggatcggcgggggggacccgcggggtgaccggcggcaggagccgccaccatggagttccgccaggaggagtttcggaagctagcgggtcgtgctctcgggaagctgcaccgccttctggagaagcggcaggaaggcgcggagacgctggagctgagtgcggatgggcgcccggtgaccacgcagacccgggacccgccggtggtggactgcacctgcttcggcctccctcgccgctacattatcgccatcatgagtggtctgggcttctgcatcagctttggcatccgctgcaacctgggcgtggccatcgtctccatggtcaataacagcacgacccaccgcgggggccacgtggtggtgcagaaagcccagttcagctgggatccagagactgtcggcctcatacacggctcctttttctggggctacattgtcactcagattccaggaggatttatctgtcaaaaatttgcagccaacagagttttcggctttgctattgtggcaacatccactctaaacatgctgatcccctcagctgcccgcgtccactatggctgtgtcatcttcgtgaggatcctgcaggggttggtagagggggtcacataccccgcctgccatgggatctggagcaaatgggccccacccttagaacggagtcgcctggcgacgacagccttttgtggttcctatgctggggcggtggtcgcgatgcccctcgccggggtccttgtgcagtactcaggatggagctctgttttctacgtctacggcagcttcgggatcttctggtacctgttctggctgctcgtctcctacgagtcccccgcgctgcacccaagcatctcggaggaggagcgcaagtacatcgaggacgccatcggagagagcgcgaaactcatgaaccccctcacgaagtttagcactccctggcggcgcttcttcacgtctatgccagtctatgccatcatcgtggccaacttctgccgcagctggacgttctacctgctgctcatctcccagcccgcctacttcgaagaagtgttcggcttcgagatcagcaaggtaggcctggtgtccgcgctgccccacctggtcatgaccatcatcgtgcccatcggcggccagatcgcggacttcctgcggagccgccgcatcatgtccaccaccaacgtgcgcaagttgatgaactgcggaggcttcggcatggaagccacgctgctgttggtggtcggctactcgcactccaagggcgtggccatctccttcctggtcctagccgtgggcttcagcggcttcgccatctctgggttcaacgtgaaccacctggacatagcccggcgctacgccagcatcctcatgggcatctccaacggcgtgggcacactgtcgggcatggtgtgccccatcatcgtgggggccatgactaagcacaagactcgggaggagtggcagtacgtgttcctaattgcctccctggtgcactatggaggtgtcatcttctacggggtctttgcttctggagagaagcagccgtgggcagagcctgaggagatgagcgaggagaagtgtggcttcgttggccatgaccagctggctggcagtgacgacagcgaaatggaggatgaggctgagcccccgggggcaccccctgcacccccgccctcctatggggccacacacagcacatttcagccccccaggcccccaccccctgtccgggactactgaccatgtgcctcccactgaatggcagtttccaggacctccattccactcatctctggcctgagtgacagtgtcaaggaaccctgctcctctctgtcctgcctcaggcctaagaagcactctcccttgttcccagtgctgtcaaatcctctttccttcccaattgcctctcaggggtagtgaagctgcagactgacagtttcaaggatacccaaattcccctaaaggttccctctccacccgttctgcctcagtggtttcaaatctctcctttcagggctttatttgaatggacagttcgacctcttactctctcttgtggttttgaggcacccacaccccccgctttcctttatctccagggactctcaggctaacctttgagatcactcagctcccatctcctttcagaaaaattcaaggtcctcctctagaagtttcaaatctctcccaactctgttctgcatcttccagattggtttaaccaattacttgtccccgccattccagggattgattctcaccagcgtttctgatggaaaatggcggtttcaagtccccgattccgtgcccacttcacatctcccctaccagcagattctgcgaaagcaccaaatttctcaagaccctcttctccctagcttagcataatgtctggggaaacaaccaaaatcgcaattttaacaatatgcctctctacccccgtgcactttttctgacatggttttcaggtctaaatagtggctgctccagtccatgaactcaaaggtttgaagctaccaccattgaactcccccatggtggtttcatgatgccccctccccaattcctcgcactttattctcctgggtggtttcgaactaccctgtttctcagtggccatttgttgtgtccctcaggggcttaatgactcaaaatctgggatccttcccctctcagacacccctctttcagcttagaatttggggaacctatagagaagtcacagaatcccatgaaagggaatggggcaggagacaggggtgttttttcccagtgcagggttgtgtctttgtgtctctgtctctgtgactgtaaatcctgccctgccccactcccaataaaagctttggtgtataggcaaaaaaaaaaa

SEQ ID NO: 108 - DNA, 3946 bp, Homo sapiens
  misc_feature (1)..(3946): VGLUT2 (Fig. 27B)
cgtttaaaagccatcagatttgagagcaataagtcttcaaaaccgggaatttacattgtttttcagctgaccgacttccaggaaaaggactcaaccgcatctacccaaataccgtggcactgcttgcgctctttgccaccggatactccccttccaatgagactttctgattgtgtctaccaactctcctattaggaaacccgtgggttgcatgcagctattctgttgtattctcattctcactctccctcccttctctcactctcactcttgctggaggcgagccactaccattctgctgagaaggaaaagcccgcaactactttaagagattaagacaatatgcgcaatcctcgcctttcctagcaatcactatttaaatctggcaagaactgacaacagtctttgcaagaatggaatccgtaaaacaaaggattttggccccaggaaaagaggggctaaagaattttgctggaaaatcactcggccagatctacagggtgctggagaagaagcaagacaccggggagacaatcgagctgacggaggatgggaagcccctagaggtgcccgagaggaaggcgccgctgtgcgactgcacgtgcttcggcctgccccgccgctacattatcgccatcatgagcggcctgggcttctgcatctccttcggtatccgctgcaacctgggcgtggccattgtggacatggtcaacaacagcaccatccaccgcgggggcaaggtcatcaaggagaaagccaaattcaactgggacccggaaaccgtggggatgatccacggttccttcttttggggctacatcatcactcagattccgggaggctacatcgcgtctcggctggcagccaacagggttttcggagctgccatacttcttacctctaccctaaatatgctaattccatcagcagccagagtgcattatggatgtgtcatctttgtcagaatactgcagggacttgttgagggtgtgacctacccagcatgtcatgggatatggagcaaatgggccccacctctagagaggagtagactggcaaccacctccttttgtggttcctatgccggagctgtgattgcaatgcctttagctggcattcttgtgcagtacactggctggtcttcagtgttttatgtctacggaagctttggaatggtctggtacatgttttggcttttggtgtcttatgaaagtcctgcaaagcatcctactattacagatgaagaacgtaggtacatagaagaaagcattggagagagtgcaaatcttttaggtgcaatggaaaaattcaagactccatggaggaagttttttacatccatgccagtctatgcaataattgttgcaaacttctgcagaagctggactttttatttattgcttattagtcagccagcatattttgaggaagtctttggatttgaaattagcaaggttggtatgctatctgctgtgccacacttagtaatgacaattattgtgcctattgggggacaaattgcagattttctaagaagcaagcagattctttcaactacgacagtgagaaagatcatgaattgtggtggttttggcatggaagccacactgctcctggtcgttggctattctcatactagaggggtagcaatctcattcttggtacttgcagtgggattcagtggatttgctatatctggtttcaatgttaaccacttggatatcgctccaagatatgccagtatcttaatgggcatttcgaatggtgttggcacattgtcaggaatggtttgtcctatcattgttggtgcaatgacaaagaataagtcacgtgaagagtggcagtatgtcttcctgatcgctgccctagtccactatggtggagttatattttatgcaatatttgcctcaggagagaaacaaccctgggcagacccggaggaaacaagtgaagaaaaatgtggatttattcatgaagatgaactcgatgaagaaacaggggacattactcaaaattatataaattatggtaccaccaagtcttatggtgccacaacacaggccaatggaggttggcctagtggttgggaaaagaaagaggaatttgtacaaggagaagtacaagactcacatagctataaggaccgagttgattattcataacaaaactaattactggatttatttttagtgtttgtgattaaattcattgtgattgcacaaaaattttaaaaacacgtgatgtaaacttgcaagcatatcaaccaggcaagtcttgctgtaaaaatgaaaacaaaacaaacccatgaggttaccatcaagtgcaatctgtaaaattgtgaagttccatcatttccattcaagtcatccattcttgcatttgtgacttaaaggttgactggtcaaaattgtagaaacaagtagttacccattggattcatatgagctaaaactcatcactatttactaaagcacaacatctcatcctacaaaagttaagaagccaaagctacttgatcatgcaaaatgcacttatatatttgttacactgtattgcaagatagcacacagaagttggctgcgtcaagtagaggcgacatttattaagtgaaaatcatggagttgggatatctctcaattaaagaaatacattgtgaactatcagctacaaagttgtactgaataactattagaattgcataatgtgagatattttgttagtcctcaaaaggaatatcttgcagtgttttctatgaaatgcttgggcacaaacacttatttctgtgaaagagaacatgtaagttgaggggtatgcttcatgttcttccatccatttacctaatagtatgaaacagttcacatttcaataaaatcaaacttttcatgtagcgtatcacataacttttttgcaaaaaatataaaaagaaataaacttcaatgtattttttattacaactttgtactggttgtaacttgcattagaaaaaaaaaagagatatataaaccacaaagaatctaataagaaatttattatggagatatagcccttaaaatgcaatattaagaacaaagaaatagaaaatggtttagatatctttcttccttcataattaaatactatatgaaacttgtgccacagagctatatgtaatatgaaaagattaacttcatagagatattgtaagtaggtaattttattatttaaagtcctattaagaaatatttgtcttaaatatataggacaatacattatattaaaatggtctctctctatatatatctgtatatcttatacatgtccatacacagaaacataataaacaatcttcacacgaaaccaaaaatagcatacacctaatgttgggttagggaattgcaatttctactttcatagagtcatagaattttaggtggggaagaggcattttgcttgtcatttcttaatataactcaacaagaattgcaacatttgtgtaccaagcaataagtgcaatgcataaaatttcctgtctgtatattaccttcattttgcttgtagtagctgtttgggtggttggaataattttatttttcttttaaaaaagctaacatcagacccctttataatgtcctaaaattatgataatacatttcccaattcaactcaaaatattattggtgtattttgtctattctggatatttgatctgtttaatgtactgtgctagtgactggaggccctgctactgcaaatataaaacctaaagtttgtttaaaaaaatgcaaatcattctttaccttaagaaaaaaaaaataccctttgctttgtgcctcaaagtgatgtaatgtgatcacagcttttgttgtgttgaatgaaaatatgtggactgtcattttgttgcagcaaaaaagtgttaataaaatgctctatttatccttttttaaaaaaaaaaaaaaaaaaaaaaaaa

SEQ ID NO: 109 - DNA, 3838 bp, Homo sapiens
  misc_feature (1)..(3838): VGLUT3 (Fig. 27C)
tcttggaagatccgagctgggtttcatctcctttttgattttgagtagttccctccacgagaactgacttccaggtgttcaccaagggaaacaaggtggttctcacactggaaatgaggaaggatgacagtttttgagactgactgttaacggctcagaggtgcccctcattcaaaatgccttttaaagcatttgataccttcaaagaaaaaattctgaaacctgggaaggaaggagtgaagaacgccgtgggagattctttgggaattttacaaagaaaaatcgatgggacaactgaggaagaagataacattgagctgaatgaagaaggaaggccggtgcagacgtccaggccaagccccccactctgcgactgccactgctgcggcctccccaagcgttacatcattgctatcatgagtgggctgggattctgcatttcctttgggatccggtgcaatcttggagttgccattgtggaaatggtcaacaatagcaccgtatatgttgatggaaaaccggaaattcagacagcacagtttaactgggatccagaaacagtgggccttatccatggatcttttttctggggctatattatgacacaaattccaggtggtttcatttcaaacaagtttgctgctaacagggtctttggagctgccatcttcttaacatcgactctgaacatgtttattccctctgcagccagagtgcattacggatgcgtcatgtgtgtcagaattctgcaaggtttagtggagggtgtgacctacccagcctgccatgggatgtggagtaagtgggcaccacctttggagagaagccgactggccacaacctctttttgtggttcctatgcaggggcagtggttgccatgcccctggctggggtgttggtgcagtacattggatggtcctctgtcttttatatttatggcatgtttgggattatttggtacatgttttggctgttgcaggcctatgagtgcccagcagctcatccaacaatatccaatgaggagaagacctatatagagacaagcataggagagggggccaacgtggttagtctaagtaaatttagtaccccatggaaaagatttttcacatctttgccggtttacgcaatcattgtggcaaatttttgcagaagctggaccttttatttgctcctcataagtcagcctgcttattttgaagaggtctttggatttgcaataagtaaggtgggtctcttgtcagcagtcccacacatggttatgacaatcgttgtacctattggaggacaattggctgattatttaagaagcagacaaattttaaccacaactgctgtcagaaaaatcatgaactgtggaggttttggcatggaggcaaccttactcctggtggttggcttttcgcataccaaaggggtggctatctcctttctggtacttgctgtaggatttagtggcttcgctatttcaggttttaatgtcaaccacctggacattgccccacgctatgccagcattctcatggggatctcaaacggagtgggaaccctctctggaatggtctgtcccctcattgtcggtgcaatgaccaggcacaagacccgtgaagaatggcagaatgtgttcctcatagctgccctggtgcattacagtggtgtgatcttctatggggtctttgcttctggggagaaacaggagtgggctgacccagagaatctctctgaggagaaatgtggaatcattgaccaggacgaattagctgaggagatagaactcaaccatgagagttttgcgagtcccaaaaagaagatgtcttatggagccacctcccagaattgtgaagtccagaagaaggaatggaaaggacagagaggagcgacccttgatgaggaagagctgacatcctaccagaatgaagagagaaacttctcaactatatcctaatgtctgagaggcacttctgtcttctccttactttagaaacagaaagtatccatacctattgcctttcttgtagcccagcttgccagaggtccaaatattgggaggggagaagatctaaccagcaacagggaaaagagaaatattatctttcaatgacatgtataggtaaggagctgcgctcagttgataacatagttgataatacatattttttgaattgacagttgacccttctctcaaagagctaaacttattcagaaaggaatgactagaagaaaaaggagacaataccatgttgttcaaagaaacattgaaggaaattgggatgtttggccagaaggaatgtaaacagtagtagtagctgccaccacatctctagggtagccatgcagaggagggcttcatattcccaataaaccccacgttgtggcaggtgctttataaacactcttatttaatctccacacctttatgacacacatttcttatccccattttacaaccaaggcatctaaagcaacaagaaatgaacttgcccaaggtcatctgccagggtcagtgctgagactgttgaagctctcaataggtggcagttttagggaagatttccattcagtgtagggaagacatttgtaataatgaaaactgaaaatggagtaattgtgagtaactcaccactttagcaggtgttggggaagggaaacatttgggttgatgaggcagaggggattcaaatgtgtgagaggctagattcaaagaccctcagtgttctatgttatctgaagagtcaaatggttttgtgactccatagtttttaaagtaataagggtcaaagactacatcagagattcaaataggtttttaaagaaaagctaagcaagagagccaaatttttagaaatctgatggtcaaaatagctgaaagcagtaaacaagagattggctattaaatttcaactttccataatattaagaatgtagctaaatgatgtcccaaactacttacaaacttttaagacatttaataatttaagaagtaggttcatgtgttttcttaggtaaagttcttctgaaagaattttctatttttaaaaaatgtatctctttagccttttctgctggagattatattaggaagtttcatcagattgtataaaattatgattttgtatcaaaagtattcatgatgactctatttggaatgatattcagggaaatcacaataatatagcagtagttatacagagaaatactacaatgaaaacatttggggcaattagacctacagttactgttgaaaaattcacctttgattgcataaggcaattacatggatacttttagatatatttaaaattttaacattggcatctaaagtgttatttgaaaataaaattattttcctgttcattgattttaaacattttattcctactttcagaagaaaaatataatacggaaaaaattatagatttacttgtagcttattattgtaaagtgttttttttttttttctaatttctcccacatgtatttctggtccccagtgatactagctgagttgtagtgtattttataaatggaataatcttggggaaaaattgcgattcttcattaaataatattctttatgtcactagcatacaatttatgttagtagacatctttaaatctctttaatgagtgaatccatgcaagccccataaaacagttcctagcatgcagaaaatgcccacgtaaatagctgtcatcatcattatcttttaacattttgggggactttccagttgaaaagaaaacatgctatgtcatttttatccattatccctggaacttattgtgaaagttgtgctgttttctaagtaaaataaaaaataaaaaattagcaattta

SEQ ID NO: 110 - RNA, 21 nt, Unknown
  Target sequence of a mRNA of a member of the VGLUT-Family, being complementary to a dsRNA sequence according to the invention (claim 21).
aaguguacuuuaggcaaaggg

SEQ ID NO: 111 - DNA, 21 bp, Artificial sequence
  Sense strand of dsRNA, which is complementary to the afore disclosed target sequence (claim 22).
  misc_feature (20)..(20): T = dT (desoxy Thymidin)
  misc_feature (21)..(21): T = dT (desoxy Thymidin)
guguacuuuaggcaaagggtt

SEQ ID NO: 112 - DNA, 21 bp, Artificial sequence
  Antisense strand of dsRNA, which is complementary to the afore disclosed target sequence (claim 22).
  misc_feature (20)..(20): T = dT (desoxy Thymidin)
  misc_feature (21)..(21): T = dT (desoxy Thymidin)
cccuuugccuaaaguacactt